SEQUENCE LISTING (125 sequences)

SEQ ID NO: 1 - DNA, 409 bp, Rattus rattus
  source 1..409: mol_type = genomic DNA
  CDS 58..>409: protein_id = 2; translation = QVILKESGPGMLQPSKTLSLTCSFSGFSLSTSGMVVSWIRQPSG KSLEWLAAIDWDGEKYYNPSLKSRLTVSKDTSNTQVFLQITTVDIADTATYYCARSGN YFEYWGQGVIVXVSS
  V_region 58..132
  V_segment 133..162
  V_region 163..213
  V_segment 214..234
  V_region 235..348
  V_segment 349..375
  J_segment 376..409
  sig_peptide 1..57
atggacaggcttacttcctcattcctgctgctgcttgtccctgcatatgtcttgtcccaggttattctgaaagagtctggtcctgggatgttacagccctccaagaccctaagcctgacttgctctttttctgggttttcactgagcacttctggtatggttgtgagctggattcgtcagccttcagggaagagtctcgagtggctggcagccattgattgggatggtgagaagtactacaacccatctctgaaaagcaggctcacagtctccaaggacacctccaacacccaggtattcctccagatcaccactgtggacattgcagatactgccacatattactgtgctcggagtggtaactactttgagtactggggccaaggagtcatagtcwcagtctcctcag

SEQ ID NO: 2 - protein, 117 aa, Rattus rattus
QVILKESGPGMLQPSKTLSLTCSFSGFSLSTSGMVVSWIRQPSGKSLEWLAAIDWDGEKYYNPSLKSRLTVSKDTSNTQVFLQITTVDIADTATYYCARSGNYFEYWGQGVIVXVSS

SEQ ID NO: 3 - protein, 25 aa, Rattus rattus
QVILKESGPGMLQPSKTLSLTCSFS

SEQ ID NO: 4 - protein, 10 aa, Rattus rattus
GFSLSTSGMV

SEQ ID NO: 5 - protein, 17 aa, Rattus rattus
VSWIRQPSGKSLEWLAA

SEQ ID NO: 6 - protein, 7 aa, Rattus rattus
IDWDGEK

SEQ ID NO: 7 - protein, 38 aa, Rattus rattus
YYNPSLKSRLTVSKDTSNTQVFLQITTVDIADTATYYC

SEQ ID NO: 8 - protein, 9 aa, Rattus rattus
ARSGNYFEY

SEQ ID NO: 9 - protein, 11 aa, Rattus rattus
WGQGVIVXVSS

SEQ ID NO: 10 - DNA, 382 bp, Rattus rattus
  source 1..382: mol_type = genomic DNA
  sig_peptide 1..60
  CDS 61..>382: protein_id = 11; translation = DNQMTQSPASLSASLGETVTIECRTSEDIYNALAWYQQKPGKSP QLLIYDANRLHIGVPSRFSGSGSGTQFSLKINSLQSEDVASYFCQQYYDYPNTFGAGT KLELN
  V_region 61..138
  V_segment 139..156
  V_region 157..207
  V_segment 208..216
  V_region 217..324
  V_segment 325..351
  J_segment 352..382
atgggtgtccccactcagctcctggggttgttgctactgtggattacagatgtcatatgtgacaaccagatgacacagtctccagcttccctgtctgcatctctgggagaaactgtcaccatcgaatgtcgaacaagcgaggacatttacaatgctttagcatggtatcagcagaagccagggaaatctcctcagctcctgatctatgatgcaaataggttgcatattggggtcccatcacggttcagtggcagtggatctggtacacagttttctctcaagataaacagcctgcaatctgaagatgtcgcaagttatttctgtcaacagtattacgattatccgaacacgtttggagctgggaccaagctggagctgaacc

SEQ ID NO: 11 - protein, 107 aa, Rattus rattus
DNQMTQSPASLSASLGETVTIECRTSEDIYNALAWYQQKPGKSPQLLIYDANRLHIGVPSRFSGSGSGTQFSLKINSLQSEDVASYFCQQYYDYPNTFGAGTKLELN

SEQ ID NO: 12 - protein, 26 aa, Rattus rattus
DNQMTQSPASLSASLGETVTIECRTS

SEQ ID NO: 13 - protein, 6 aa, Rattus rattus
EDIYNA

SEQ ID NO: 14 - protein, 17 aa, Rattus rattus
LAWYQQKPGKSPQLLIY

SEQ ID NO: 15 - skipped

SEQ ID NO: 16 - protein, 36 aa, Rattus rattus
RLHIGVPSRFSGSGSGTQFSLKINSLQSEDVASYFC

SEQ ID NO: 17 - protein, 9 aa, Rattus rattus
QQYYDYPNT

SEQ ID NO: 18 - protein, 10 aa, Rattus rattus
FGAGTKLELN

SEQ ID NO: 19 - DNA, 424 bp, Mus musculus
  source 1..424: mol_type = genomic DNA
  CDS 58..>424: protein_id = 20; translation = EVHLQQSGPELVKPGASVKMSCKASGYTFTSYVIHWVKQRPGQG LEWIGNIYPYNDDTNYNEKFKGKATLTSDKSSSTAYMELSSLTSEESAVYFCTRERAT YGDLAWFAYWGQGTLVTVSA
  sig_peptide 1..57
  V_region 58..132
  V_segment 133..156
  V_region 157..207
  V_segment 208..231
  V_region 232..345
  V_segment 346..390
  J_segment 391..424
atggaatggagctggatatttctctttctcctgtcaggaactgcaggtgtccactctgaggtccacctgcagcagtctggacctgaactggtaaagcctggggcttcagtgaagatgtcctgcaaggcttctggatacacattcactagctatgttatacactgggtgaagcagaggcctgggcagggccttgagtggattggaaatatttatccttacaatgatgatactaactacaatgagaagttcaaaggcaaggccacactgacttcagacaaatcctccagcacagcctacatggagctcagcagcctgacctctgaggagtctgcggtctatttctgtacaagagagagggctacctatggtgaccttgcctggtttgcttactggggccaagggactctggtcactgtctctgcag

SEQ ID NO: 20 - protein, 122 aa, Mus musculus
EVHLQQSGPELVKPGASVKMSCKASGYTFTSYVIHWVKQRPGQGLEWIGNIYPYNDDTNYNEKFKGKATLTSDKSSSTAYMELSSLTSEESAVYFCTRERATYGDLAWFAYWGQGTLVTVSA

SEQ ID NO: 21 - protein, 25 aa, Mus musculus
EVHLQQSGPELVKPGASVKMSCKAS

SEQ ID NO: 22 - protein, 8 aa, Mus musculus
GYTFTSYV

SEQ ID NO: 23 - protein, 17 aa, Mus musculus
IHWVKQRPGQGLEWIGN

SEQ ID NO: 24 - protein, 8 aa, Mus musculus
IYPYNDDT

SEQ ID NO: 25 - protein, 38 aa, Mus musculus
NYNEKFKGKATLTSDKSSSTAYMELSSLTSEESAVYFC

SEQ ID NO: 26 - protein, 15 aa, Mus musculus
TRERATYGDLAWFAY

SEQ ID NO: 27 - protein, 11 aa, Mus musculus
WGQGTLVTVSA

SEQ ID NO: 28 - DNA, 394 bp, Mus musculus
  source 1..394: mol_type = genomic DNA
  sig_peptide 1..60
  CDS 61..>394: protein_id = 29; translation = NIVLTQSPASLAVSLGQRATISCRASENIDSYGNSFMHWYQQKP GQPPKLLIYLASILESGVPARFSGSGSRTDFTLTIDPVEADDAATYYCQQNNEDPYTF GGGTKLEIK
  V_region 61..138
  V_segment 139..168
  V_region 169..219
  V_segment 220..228
  V_region 229..336
  V_segment 337..363
  J_segment 364..394
atggagacagacacactcctgctatgggtgctgctgctctgggttccaggttccacaggtaacattgtgctgacccaatctccagcttctttggctgtgtctctagggcagagggccaccatatcctgcagagccagtgaaaatattgatagttatggcaatagttttatgcactggtaccagcagaaaccaggacagccacccaaactcctcatctatcttgcatccatcctagaatctggggtccctgccaggttcagtggcagtgggtctaggacagacttcaccctcaccattgatcctgtggaggctgatgatgctgcaacctattactgtcagcaaaataatgaggatccgtacacgttcggaggggggaccaagctggaaataaaac

SEQ ID NO: 29 - protein, 111 aa, Mus musculus
NIVLTQSPASLAVSLGQRATISCRASENIDSYGNSFMHWYQQKPGQPPKLLIYLASILESGVPARFSGSGSRTDFTLTIDPVEADDAATYYCQQNNEDPYTFGGGTKLEIK

SEQ ID NO: 30 - protein, 26 aa, Mus musculus
NIVLTQSPASLAVSLGQRATISCRAS

SEQ ID NO: 31 - protein, 10 aa, Mus musculus
ENIDSYGNSF

SEQ ID NO: 32 - protein, 17 aa, Mus musculus
MHWYQQKPGQPPKLLIY

SEQ ID NO: 33 - skipped

SEQ ID NO: 34 - protein, 36 aa, Mus musculus
ILESGVPARFSGSGSRTDFTLTIDPVEADDAATYYC

SEQ ID NO: 35 - protein, 9 aa, Mus musculus
QQNNEDPYT

SEQ ID NO: 36 - protein, 10 aa, Mus musculus
FGGGTKLEIK

SEQ ID NO: 37 - DNA, 415 bp, Rattus rattus
  source 1..415: mol_type = genomic DNA
  sig_peptide 1..57
  CDS 58..>415: protein_id = 38; translation = QVQLKESGPGLVQPSQTLSLTCTVSGFSLDNYNVHWVRQPTGGG LEWLGGIWTDGITDYTSPLRSRLSISRDTSKSQVFLKMKSLQTEDIATYYCARSYYYS LYYFDYWGQGVMVTVSS
  V_region 58..132
  V_segment 133..156
  V_region 157..207
  V_segment 208..228
  V_region 229..342
  V_segment 343..381
  J_segment 382..415
atggctgtcctggtgctgttgctctgcctggtgacatttccaacctgtgtcctgtcccaggtgcagctgaaggagtcaggacctggtctggtgcagccctcacagaccctgtccctcacctgcactgtctctggattctcactagacaactacaatgtgcactgggttcgacagcctacaggaggaggtctggagtggttgggaggaatttggactgatggaatcacagactacacttcacctctcagatcccgactgagcatcagcagggacacctccaagagccaggttttcttaaagatgaagagtctgcaaactgaggacatagccacttactactgtgccaggtcttattactattccctatactactttgattactggggccaaggagtcatggtcacagtctcctcag

SEQ ID NO: 38 - protein, 119 aa, Rattus rattus
QVQLKESGPGLVQPSQTLSLTCTVSGFSLDNYNVHWVRQPTGGGLEWLGGIWTDGITDYTSPLRSRLSISRDTSKSQVFLKMKSLQTEDIATYYCARSYYYSLYYFDYWGQGVMVTVSS

SEQ ID NO: 39 - protein, 25 aa, Rattus rattus
QVQLKESGPGLVQPSQTLSLTCTVS

SEQ ID NO: 40 - protein, 8 aa, Rattus rattus
GFSLDNYN

SEQ ID NO: 41 - protein, 17 aa, Rattus rattus
VHWVRQPTGGGLEWLGG

SEQ ID NO: 42 - protein, 7 aa, Rattus rattus
IWTDGIT

SEQ ID NO: 43 - protein, 38 aa, Rattus rattus
DYTSPLRSRLSISRDTSKSQVFLKMKSLQTEDIATYYC

SEQ ID NO: 44 - protein, 13 aa, Rattus rattus
ARSYYYSLYYFDY

SEQ ID NO: 45 - protein, 11 aa, Rattus rattus
WGQGVMVTVSS

SEQ ID NO: 46 - DNA, 379 bp, Rattus rattus
  source 1..379: mol_type = genomic DNA
  sig_peptide 1..60
  CDS 61..>379: protein_id = 47; translation = DIQMTQSPASLSASLGETVTIECRASEDIYSNLAWYQQKPGDSP QLLIYDAHRLAEGVPSRFSGSGSGTQYSLKINSLHSEDVASYFCQQYDNYNTFGVGTK LDLK
  V_region 61..138
  V_segment 139..156
  V_region 157..207
  V_segment 208..216
  V_region 217..324
  V_segment 325..348
  J_segment 349..379
atgggtgtccccactcagctcctggggttgttgctactgtggattacagatgccatatgtgacatccagatgacacagtctccagcttccctgtctgcatctctgggagaaactgtcaccatcgaatgtcgagcaagtgaggacatttacagtaatttagcgtggtatcagcagaaaccaggggactctcctcagctcctgatctatgatgcacataggttggcagagggggtcccatcacggttcagtggcagtggatctggcacacagtattctctaaagataaacagcctgcactctgaagatgtcgcaagttatttctgtcaacaatatgacaattataacacgtttggagttgggaccaagctggacctgaaac

SEQ ID NO: 47 - protein, 106 aa, Rattus rattus
DIQMTQSPASLSASLGETVTIECRASEDIYSNLAWYQQKPGDSPQLLIYDAHRLAEGVPSRFSGSGSGTQYSLKINSLHSEDVASYFCQQYDNYNTFGVGTKLDLK

SEQ ID NO: 48 - protein, 26 aa, Rattus rattus
DIQMTQSPASLSASLGETVTIECRAS

SEQ ID NO: 49 - protein, 6 aa, Rattus rattus
EDIYSN

SEQ ID NO: 50 - protein, 17 aa, Rattus rattus
LAWYQQKPGDSPQLLIY

SEQ ID NO: 51 - skipped

SEQ ID NO: 52 - protein, 36 aa, Rattus rattus
RLAEGVPSRFSGSGSGTQYSLKINSLHSEDVASYFC

SEQ ID NO: 53 - protein, 8 aa, Rattus rattus
QQYDNYNT

SEQ ID NO: 54 - protein, 10 aa, Rattus rattus
FGVGTKLDLK

SEQ ID NO: 55 - DNA, 406 bp, Rattus rattus
  source 1..406: mol_type = genomic DNA
  sig_peptide 1..57
  CDS 58..>406: protein_id = 56; translation = QVQLKESGPGLVQPSQTLSLTCTVSGFSLTNYHVHWVRQPPGKG LEWMGVMWIDGDTSYNSALRSRLSISRDTSKSQVFLKMSSLQTEDTATYYCAREGLNW FAYWGRGTLVTVSS
  V_region 58..132
  V_segment 133..156
  V_region 157..207
  V_segment 208..228
  V_region 229..342
  V_segment 343..372
  J_segment 373..406
atggctgtcctggtgctgttgctctgcctgatgacatttccaagctgtgtcctgtcccaggtgcagctgaaggagtcaggacctggcctggtgcagccctcacagaccctgtctctcacctgcactgtctctgggttctcattaaccaattatcatgtgcactgggttcgacagcctccaggaaaaggtctggagtggatgggagtaatgtggattgatggcgacacatcatataattcagctctcagatcccgactgagcatcagcagggacacctccaagagccaagttttcttaaaaatgagcagtctgcagactgaagacacagccacttactactgtgccagagagggtctcaattggtttgcttactggggccgaggcactctggtcactgtctcttcag

SEQ ID NO: 56 - protein, 116 aa, Rattus rattus
QVQLKESGPGLVQPSQTLSLTCTVSGFSLTNYHVHWVRQPPGKGLEWMGVMWIDGDTSYNSALRSRLSISRDTSKSQVFLKMSSLQTEDTATYYCAREGLNWFAYWGRGTLVTVSS

SEQ ID NO: 57 - protein, 25 aa, Rattus rattus
QVQLKESGPGLVQPSQTLSLTCTVS

SEQ ID NO: 58 - protein, 8 aa, Rattus rattus
GFSLTNYH

SEQ ID NO: 59 - protein, 17 aa, Rattus rattus
VHWVRQPPGKGLEWMGV

SEQ ID NO: 60 - protein, 7 aa, Rattus rattus
MWIDGDT

SEQ ID NO: 61 - protein, 38 aa, Rattus rattus
SYNSALRSRLSISRDTSKSQVFLKMSSLQTEDTATYYC

SEQ ID NO: 62 - protein, 10 aa, Rattus rattus
AREGLNWFAY

SEQ ID NO: 63 - protein, 11 aa, Rattus rattus
WGRGTLVTVSS

SEQ ID NO: 64 - DNA, 382 bp, Rattus rattus
  source 1..382: mol_type = genomic DNA
  sig_peptide 1..60
  CDS 61..>382: protein_id = 65; translation = DIQMTQSPASLSVSLEEVVTITCKASQGIDDYLSWYQQKPGKSP QLLISDVTSLADGVPSRFSGSRSGTQYSLKISRPQVDDSGIYYCLQSYSTPYTFGAGT KLELK
  V_region 61..138
  V_segment 139..156
  V_region 157..207
  V_segment 208..216
  V_region 217..324
  V_segment 325..351
  J_segment 352..382
atgaacgtgcccactcaactccttgggttgctgctactctggcttacaggtggtaaatgtgacatccagatgacacagtctcctgcctccctgtctgtttctctggaagaagttgtcaccatcacctgcaaggcaagccagggcattgatgattacttatcatggtatcagcagaaaccagggaaatctcctcagctcctgatctctgatgtaaccagcttggcagatggggtcccatcacggttcagcggcagtagatctggcacacagtattctcttaagatcagcagaccacaggttgatgattctggaatctattactgtctacagagttacagtactccgtacacgtttggagctgggaccaagctggaactgaaac

SEQ ID NO: 65 - protein, 107 aa, Rattus rattus
DIQMTQSPASLSVSLEEVVTITCKASQGIDDYLSWYQQKPGKSPQLLISDVTSLADGVPSRFSGSRSGTQYSLKISRPQVDDSGIYYCLQSYSTPYTFGAGTKLELK

SEQ ID NO: 66 - protein, 26 aa, Rattus rattus
DIQMTQSPASLSVSLEEVVTITCKAS

SEQ ID NO: 67 - protein, 6 aa, Rattus rattus
QGIDDY

SEQ ID NO: 68 - protein, 17 aa, Rattus rattus
LSWYQQKPGKSPQLLIS

SEQ ID NO: 69 - skipped

SEQ ID NO: 70 - protein, 36 aa, Rattus rattus
SLADGVPSRFSGSRSGTQYSLKISRPQVDDSGIYYC

SEQ ID NO: 71 - protein, 9 aa, Rattus rattus
LQSYSTPYT

SEQ ID NO: 72 - protein, 10 aa, Rattus rattus
FGAGTKLELK

SEQ ID NO: 73 - DNA, 409 bp, Rattus rattus
  source 1..409: mol_type = genomic DNA
  sig_peptide 1..57
  CDS 58..>409: protein_id = 74; translation = QVTLKESGPGMLQPSKTVSLTCSFSGFSMSTSGMVVSWIRQPSG KSLEWLAAIDWDGEKYYNPSLKSRLTVSKDTSNTQVFLKISSVDIADSATYYCARSGN YFDYWGQGVMVTVSS
  V_region 58..132
  V_segment 133..162
  V_region 163..213
  V_segment 214..234
  V_region 235..348
  V_segment 349..375
  J_segment 376..409
atggacaggcttacttcctcattcctgctgctgcttgtccctgcatatgtcttgtcccaggttactctgaaagagtctggtcctgggatgttacagccctccaagaccgtaagcctgacttgctctttttctgggttttcaatgagcacttctggtatggttgtgagctggattcgtcagccttcagggaagagtctcgagtggctggcagccattgattgggatggtgaaaagtactacaacccatctctgaaaagcaggctcacagtctccaaggacacctccaacacccaagtgttcctcaagatcagcagtgtggacattgcagatagtgccacatactactgtgctcggagtggtaactactttgattactggggccaaggagtcatggtcacagtctcctcag

SEQ ID NO: 74 - protein, 117 aa, Rattus rattus
QVTLKESGPGMLQPSKTVSLTCSFSGFSMSTSGMVVSWIRQPSGKSLEWLAAIDWDGEKYYNPSLKSRLTVSKDTSNTQVFLKISSVDIADSATYYCARSGNYFDYWGQGVMVTVSS

SEQ ID NO: 75 - protein, 25 aa, Rattus rattus
QVTLKESGPGMLQPSKTVSLTCSFS

SEQ ID NO: 76 - protein, 10 aa, Rattus rattus
GFSMSTSGMV

SEQ ID NO: 77 - protein, 17 aa, Rattus rattus
VSWIRQPSGKSLEWLAA

SEQ ID NO: 78 - protein, 7 aa, Rattus rattus
IDWDGEK

SEQ ID NO: 79 - protein, 38 aa, Rattus rattus
YYNPSLKSRLTVSKDTSNTQVFLKISSVDIADSATYYC

SEQ ID NO: 80 - protein, 9 aa, Rattus rattus
ARSGNYFDY

SEQ ID NO: 81 - protein, 11 aa, Rattus rattus
WGQGVMVTVSS

SEQ ID NO: 82 - DNA, 322 bp, Rattus rattus
  source 1..322: mol_type = genomic DNA
  CDS 1..322: protein_id = 83; translation = DIQMTQSPASLSASLGETVTIECRASEDIYNGLVWYQQKPGKSP QLLIYDANRLHTGVPSRFSGGGSGTQYSLKINSLQSEDVASYFCQQYYDYPNTFGAGT KLELK
gacatccagatgacacagtctccagcttccctgtctgcatctctgggagaaactgtcaccatcgaatgtcgagcaagtgaggacatttacaatggtttagtatggtatcagcagaagccagggaaatctcctcagctcctgatctatgatgcaaatcgcttgcatactggggtcccatcacggttcagtggcggtggatctggtacacagtattctctcaagataaacagcctgcaatctgaagatgtcgcaagttatttctgtcaacagtactacgattatccgaacacgtttggagctgggaccaagctggagctgaaac

SEQ ID NO: 83 - protein, 107 aa, Rattus rattus
DIQMTQSPASLSASLGETVTIECRASEDIYNGLVWYQQKPGKSPQLLIYDANRLHTGVPSRFSGGGSGTQYSLKINSLQSEDVASYFCQQYYDYPNTFGAGTKLELK

SEQ ID NO: 84 - protein, 26 aa, Rattus rattus
DIQMTQSPASLSASLGETVTIECRAS

SEQ ID NO: 85 - protein, 6 aa, Rattus rattus
EDIYNG

SEQ ID NO: 86 - protein, 17 aa, Rattus rattus
LVWYQQKPGKSPQLLIY

SEQ ID NO: 87 - skipped

SEQ ID NO: 88 - protein, 36 aa, Rattus rattus
RLHTGVPSRFSGGGSGTQYSLKINSLQSEDVASYFC

SEQ ID NO: 89 - protein, 9 aa, Rattus rattus
QQYYDYPNT

SEQ ID NO: 90 - protein, 10 aa, Rattus rattus
FGAGTKLELK

SEQ ID NO: 91 - protein, 447 aa, synthetic construct
QVILKESGPGMLQPSKTLSLTCSFSGFSLSTSGMVVSWIRQPSGKSLEWLAAIDWDGEKYYNPSLKSRLTVSKDTSNTQVFLQITTVDIADTATYYCARSGNYFEYWGQGVIVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 214 aa, synthetic construct
DNQMTQSPASLSASLGETVTIECRTSEDIYNALAWYQQKPGKSPQLLIYDANRLHIGVPSRFSGSGSGTQFSLKINSLQSEDVASYFCQQYYDYPNTFGAGTKLELNRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 447 aa, synthetic construct
QVQLQESGPGLVKPSQTLSLTCSFSGFSLSTSGMVVSWIRQPPGKALEWLAAIDWEGEKYYNPSLKSRLTVTKDTSNTQVVLTMTNMDPVDTATYYCARSGNYFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 214 aa, synthetic construct
DNQMTQSPSSLSASVGDRVTITCRTSEDIYNALAWYQQKPGKAPQLLIYDANRLHIGVPDRFSGSGSGTDFTLTISSLQAEDVAVYYCQQYYDYPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 447 aa, synthetic construct
QVQLQESGPGLVKPSQTLSLTCSFSGFSLSTSGMVVSWIRQPPGKALEWLAAIDWEGEKYYNPSLKSRLTVTKDTSKNQVVLTMTNMDPVDTATYYCARSGNYFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 447 aa, synthetic construct
QITLKESGPTLVKPTQTLTLTCTFSGFSLSTSGMVVGWIRQPPGKALEWLAAIDWEGEKYYSPSLKSRLTITKDTSKNQVVLTMTNMDPVDTATYYCARSGNYFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 214 aa, synthetic construct
DIQMTQSPSSLSASVGDRVTITCRTSEDIYNALAWYQQKPGKAPQLLIYDANRLHIGVPSRFSGSGSGTDFTLTISSLQPEDFATYYCQQYYDYPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 447 aa, synthetic construct
QVQLQESGPGLVKPSQTLSLTCSFSGFSLSTSGMVVSWIRQPPGKALEWLAAIDWEGEKYYNPSLKSRLTVTKDTSNTQVVLTMTNMDPVDTATYYCARSGNYFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 447 aa, synthetic construct
QVQLQESGPGLVKPSQTLSLTCSFSGFSLSTSGMVVSWIRQPPGKALEWLAAIDWEGEKYYNPSLKSRLTVTKDTSKNQVVLTMTNMDPVDTATYYCARSGNYFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 447 aa, synthetic construct
QITLKESGPTLVKPTQTLTLTCTFSGFSLSTSGMVVGWIRQPPGKALEWLAAIDWEGEKYYSPSLKSRLTITKDTSKNQVVLTMTNMDPVDTATYYCARSGNYFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 25 aa, synthetic construct
QVQLQESGPGLVKPSQTLSLTCSFS

SEQ ID NO: 102 - protein, 25 aa, synthetic construct
QITLKESGPTLVKPTQTLTLTCTFS

SEQ ID NO: 103 - protein, 17 aa, synthetic construct
VSWIRQPPGKALEWLAA

SEQ ID NO: 104 - protein, 17 aa, synthetic construct
VGWIRQPPGKALEWLAA

SEQ ID NO: 105 - protein, 7 aa, synthetic construct
IDWEGEK

SEQ ID NO: 106 - protein, 38 aa, synthetic construct
YYNPSLKSRLTVTKDTSNTQVVLTMTNMDPVDTATYYC

SEQ ID NO: 107 - protein, 38 aa, synthetic construct
YYNPSLKSRLTVTKDTSKNQVVLTMTNMDPVDTATYYC

SEQ ID NO: 108 - protein, 38 aa, synthetic construct
YYSPSLKSRLTITKDTSKNQVVLTMTNMDPVDTATYYC

SEQ ID NO: 109 - protein, 11 aa, synthetic construct
WGQGVIVTVSS

SEQ ID NO: 110 - protein, 26 aa, synthetic construct
DNQMTQSPSSLSASVGDRVTITCRTS

SEQ ID NO: 111 - protein, 26 aa, synthetic construct
DIQMTQSPSSLSASVGDRVTITCRTS

SEQ ID NO: 112 - protein, 17 aa, synthetic construct
LAWYQQKPGKAPQLLIY

SEQ ID NO: 113 - protein, 36 aa, synthetic construct
RLHIGVPDRFSGSGSGTDFTLTISSLQAEDVAVYYC

SEQ ID NO: 114 - protein, 36 aa, synthetic construct
RLHIGVPSRFSGSGSGTDFTLTISSLQPEDFATYYC

SEQ ID NO: 115 - protein, 10 aa, synthetic construct
FGQGTKLEIK

SEQ ID NO: 116 - protein, 117 aa, synthetic construct
QVILKESGPGMLQPSKTLSLTCSFSGFSLSTSGMVVSWIRQPSGKSLEWLAAIDWDGEKYYNPSLKSRLTVSKDTSNTQVFLQITTVDIADTATYYCARSGNYFEYWGQGVIVTVSS

SEQ ID NO: 117 - protein, 107 aa, synthetic construct; identical to SEQ ID NO: 11

SEQ ID NO: 118 - protein, 117 aa, synthetic construct
QVQLQESGPGLVKPSQTLSLTCSFSGFSLSTSGMVVSWIRQPPGKALEWLAAIDWEGEKYYNPSLKSRLTVTKDTSNTQVVLTMTNMDPVDTATYYCARSGNYFEYWGQGTLVTVSS

SEQ ID NO: 119 - protein, 107 aa, synthetic construct
DNQMTQSPSSLSASVGDRVTITCRTSEDIYNALAWYQQKPGKAPQLLIYDANRLHIGVPDRFSGSGSGTDFTLTISSLQAEDVAVYYCQQYYDYPNTFGQGTKLEIK

SEQ ID NO: 120 - protein, 117 aa, synthetic construct
QVQLQESGPGLVKPSQTLSLTCSFSGFSLSTSGMVVSWIRQPPGKALEWLAAIDWEGEKYYNPSLKSRLTVTKDTSKNQVVLTMTNMDPVDTATYYCARSGNYFEYWGQGTLVTVSS

SEQ ID NO: 121 - protein, 117 aa, synthetic construct
QITLKESGPTLVKPTQTLTLTCTFSGFSLSTSGMVVGWIRQPPGKALEWLAAIDWEGEKYYSPSLKSRLTITKDTSKNQVVLTMTNMDPVDTATYYCARSGNYFEYWGQGTLVTVSS

SEQ ID NO: 122 - protein, 107 aa, synthetic construct
DIQMTQSPSSLSASVGDRVTITCRTSEDIYNALAWYQQKPGKAPQLLIYDANRLHIGVPSRFSGSGSGTDFTLTISSLQPEDFATYYCQQYYDYPNTFGQGTKLEIK

SEQ ID NO: 123 - protein, 7 aa, synthetic construct
  VARIANT 4: note = D or E
IDWXGEK

SEQ ID NO: 124 - protein, 447 aa, synthetic construct; identical to SEQ ID NO: 100

SEQ ID NO: 125 - protein, 199 aa, Homo sapiens
MNCVCRLVLVVLSLWPDTAVAPGPPPGPPRVSPDPRAELDSTVLLTRSLLADTRQLAAQLRDKFPADGDHNLDSLPTLAMSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRL

(19) United States
(12) Patent Application Publication
(10) Pub. No.: US 2026/0297176 A1
(43) Pub. Date: Oct. 1, 2026

(54) ANTI IL-11 ANTIBODY

(71) Applicant: VVB BIO PTE LTD, Singapore (SG)

(72) Inventors: Stuart Cook, Woodstock (GB); Sebastian SCHAEFER, Singapore (SG)

(21) Appl. No.: 19/478,544

(22) PCT Filed: Apr. 24, 2024
(86) PCT No.: PCT/SG2024/050269

§ 371 Date: Oct. 24, 2025

(30) Foreign Application Priority Data
Apr. 25, 2023 (GB) 2306051.0
Jan. 24, 2024 (GB) 2400941.7

(51) Int. Cl. C07K 16/24; A61K 39/00; G01N 33/575; G01N 33/68

(52) U.S. Cl. CPC C07K 16/244; G01N 33/57585; G01N 33/6869; A61K 2039/505; C07K 2317/34; C07K 2317/40; C07K 2317/565; C07K 2317/567; C07K 2317/94; G01N 2333/5431

(57) ABSTRACT

The present invention is concerned with improved anti-IL-11 (Interleukin 11) antibodies, isolated nucleotide molecules encoding same, vectors, host cells, compositions, and uses of same.

### FIELD OF THE INVENTION

The present invention is concerned with improved anti-IL-11 (Interleukin 11) antibodies, isolated nucleotide molecules encoding same, vectors, host cells, compositions, and uses of same.

### BACKGROUND TO THE INVENTION

Interleukin 11 (IL-11) is a cytokine, a type of signalling molecule that is involved in the regulation of the immune system and a range of other physiological processes.

Cook et al. (Annu Rev Med. 2020 Jan. 27; 71:263-276. Doi: 10.1146/annurev-med-041818-011649) discusses the role of IL-11 as a regulator of fibrosis, tissue Integrity, and stromal inflammation.

Lim W W et al (Circ Res, 2022 Mar 4;130(5):728-740, doi: 10.1161/CIRCRESAHA.121.320381, PMID: 35135328) report that inhibition of IL-11 (interleukin 11) signalling using a neutralizing antibody against IL-11RA (IL-11 receptor subunit alpha) reduces aortic pathology in murine Marfan syndrome.

Widjaja A A et al (Gastroenterology. 2019 September;157(3):777-792.e14, doi: 10.1053/i.gastro.2019.05.002) report that inhibiting IL-11 signalling reduces hepatocyte death and liver fibrosis, inflammation, and steatosis in mouse models of nonalcoholic steatohepatitis (NASH).

Widjaja A A et al (J Am Soc Nephrol, 2022 Apr;33(4):718-730, doi: 10.1681/ASN.2021040577, PMID: 35140116) report that a neutralizing IL-11 antibody improves renal function and increases lifespan in a mouse model of Alport Syndrome. Ng B et a,. (Sci Transl Med. 2019 Sep. 25; 11(511):eaaw1237, doi: 10.1126/scitranslmed.aawl237) discloses that IL-11 is a therapeutic target in idiopathic pulmonary fibrosis.

WO2017103108 discloses the treatment, prevention or alleviation of fibrosis in a subject through the administration of an agent capable of inhibiting the action of Interleukin 11 (IL-11).

WO2020225147 teaches the use of agents capable of inhibiting interleukin 11 (IL-11)-mediated signalling in a method of treating or preventing a metabolic disease.

WO2022090509 teaches methods of treating or preventing an age-related disease/condition, comprising administering a therapeutically or prophylactically effective amount of an agent capable of inhibiting interleukin 11 (IL-11)-mediated signalling to a subject.

WO2018109174 and WO2019/238882 disclose anti-IL-11 antibodies. Notably, even the best antibodies disclosed in those applications (e.g. antibody BSN-3C6/BSN-3C6-F10) had K_{D} values not less than approximately 700 pM (i.e. 7E-10 M) (as detailed below, lower K_{D} values indicate higher antibody affinity for the antigen, i.e. higher binding affinity). As detailed below, the antibodies disclosed herein have lower (better) K_{D} values.

Uses of anti-IL-11 antibodies include diagnostic uses as biomarker for cancer (Ren, C et al., Tumour Biol. 2014 November; 35(11):11467-72. Doi: 10.1007/s13277-014-2459-v).

Thus, anti-IL-11 antibodies (“IL-11 antibodies”) have a number of therapeutic and diagnostic uses. This is further evidenced in the experiments below, e.g. in Experiments 9 and 10.

### SUMMARY OF THE INVENTION

As detailed in the experiments below, new anti-IL-11 antibodies have been produced with significantly enhanced PK characteristics, including lower (better) K_{D} values. The new anti-IL-11 antibodies are grouped/categorised under the names “3F6”, “9B9”, “2B11”, “10D10”, and “3G8”.

In addition, the epitopes bound by the new anti-IL-11 antibodies have been identified, and so also disclosed are monoclonal antibodies and immunologically active (i.e. antigen binding) fragments thereof specific against those epitopes and which have similarly enhanced PK characteristics, including lower (better) K_{D} values as compared to previously identified anti-IL-11 antibodies.

Each of the antibodies disclosed herein can be said to have been “isolated and purified”. 3F6: According to a first aspect of the present invention there is provided an antibody that binds to human IL-11, the antibody comprising a heavy chain (HC) variable region (VH) sequence and a light chain (LC) variable region (VL) sequence,
(i) the heavy chain variable region sequence comprising the CDR3 and at least one additional CDR selected from the group consisting of:
   (a) HC-CDR1 comprising the amino acid sequence GFSLSTSGMV (SEQ ID NO: 4) or a variant having one, two or three amino acid substitutions therein,
   (b) HC-CDR2 comprising the amino acid sequence of IDWXGEK wherein ‘X’ is D or E (SEQ ID NO: 123) or a variant having one or two amino acid substitutions therein, and
   (c) HC-CDR3 comprising the amino acid sequence ARSGNYFEY (SEQ ID NO: 8) or a variant having one or two amino acid substitutions therein; and
(ii) the light chain variable region sequence comprising the CDR3 and at least one additional CDR selected from the group consisting of:
   (a) LC-CDR1 comprising the amino acid sequence EDIYNA (SEQ ID NO: 13) or a variant having one or two amino acid substitutions therein,
   (b) LC-CDR2 comprising the amino acid sequence DAN or a variant having one amino acid substitution therein, and
   (c) LC-CDR3 comprising the amino acid sequence QQYYDYPNT (SEQ ID NO: 17) or a variant having one or two amino acid substitutions therein.

The heavy chain variable region sequence can also be referred to as a “VH domain”. The light chain variable region sequence can also be referred to as a “VL domain”.

In certain embodiments, the VH domain comprises all three of the CDR1, CDR2 and CDR3 (i.e. HC-CDR1, HC-CDR2, and HC-CDR3). In certain embodiments, the VL domain comprises all three of the CDR1, CDR2 and CDR3 (i.e. LC-CDR1, LC-CDR2, and LC-CDR3).

In certain embodiments, the VH domain comprises all three of the CDR1, CDR2 and CDR3, and the VL domain comprises all three of the CDR1, CDR2 and CDR3.

In certain embodiments, the HC-CDR1 has one amino acid substitution. In certain embodiments, it has no substitutions.

In certain embodiments, the HC-CDR2 has the sequence IDWDGEK (SEQ ID NO: 6), which is the HC-CDR2 from the original rat mAb. In certain embodiments, the HC-CDR2 has the sequence IDWEGEK (SEQ ID NO: 105), which was generated as the result of sequence humanisation (i.e. is a synthetic sequence).

In certain embodiments, the HC-CDR2 has one amino acid substitution. In certain embodiments, it has no substitutions.

In certain embodiments, the HC-CDR3 has one amino acid substitution. In certain embodiments, it has no substitutions.

In certain embodiments, the LC-CDR1 has one amino acid substitution. In certain embodiments, it has no substitutions.

In certain embodiments, the LC-CDR2 has one amino acid substitution. In certain embodiments, it has no substitutions.

In certain embodiments, the LC-CDR3 has one amino acid substitution. In certain embodiments, it has no substitutions.

The term “antibody”, as used herein, generally refers to a proteinaceous binding molecule with immunoglobulin-like functions. Typical examples of an antibody are immunoglobulins, as well as derivatives or functional fragments thereof (immunologically active fragments) (antigen binding fragments) which still retain binding specificity.

Techniques for the production of antibodies are well known in the art (e.g. Greenfield E A, “Antibodies: A Laboratory Manual”, Cold Spring Harbor Press; Second Edition, 2013, ISBN: 1936113813). The term “antibody” may also include immunoglobulins of different classes (i.e. IgA, IgG, IgM, IgD and IgE) and subclasses (such as IgG1, IgG2, IgG3, IgG4, IgA1 and IgA2). Illustrative examples of an antibody are whole antibodies and antigen binding fragments thereof, such as Fab fragments, F(ab′)2, Fv fragments, single-chain Fv fragments (scFv), diabodies, domain antibodies, and combinations thereof. Domain antibodies may be single domain antibodies, single variable domain antibodies or immunoglobulin single variable domain having only one variable domain, which may be a heavy chain variable domain or a light chain variable domain, that specifically bind an antigen or epitope independently of other variable regions or domains. The term “antibody” also includes embodiments such as chimeric, single chain and humanized antibodies.

In certain embodiments, the antibody is not naturally occurring, i.e. is not an isolated antibody from an animal which has been generated as a result of immunisation of the animal with human IL-11 (hlL-11) or another naturally occurring antigen.

Humanized forms of nonhuman (e.g. murine or rat) antibodies are chimeric immunoglobulins, immunoglobulin chains or fragments thereof (such as Fv, Fab, Fab′, F(ab′)2 or other antigen binding sub-sequences of antibodies) which contain minimal sequence derived from non-human immunoglobulin. Humanized antibodies include human immunoglobulins (recipient antibody) in which residues from a complementarity determining region (CDR) of the recipient are replaced by residues from a CDR of a nonhuman species (donor antibody) such as mouse, rat, or rabbit having the desired specificity, affinity, and capacity. In some instances, Fv framework residues of the human immunoglobulin are replaced by corresponding non-human residues. Humanized antibodies may also comprise residues which are found neither in the recipient antibody nor in the imported CDR or framework sequences. A humanized antibody can comprise substantially all of at least one, and typically two, variable domains, in which all or substantially all of the CDR regions correspond to those of a non-human immunoglobulin and all or substantially all of the framework (FR) regions (i.e., the sequences between the CDR regions) are those of a human immunoglobulin consensus sequence. The humanized antibody optimally also will comprise at least a portion of an immunoglobulin constant region (Fc), typically that of a human immunoglobulin (Jones et al., Nature 321:522 (1986); Riechmann et al., Nature, 332:323 (1988); and Presta, Curr Op. Struct. Biol.2:593 (1992)). Antibody humanization is further detailed in e.g. Safdari Y et al. (Biotechnol Genet Eng Rev, 2013; 29:175-86, PMID: 24568279; doi: 10.1080/02648725.2013.801235), Marks C et al. (Bioinformatics 2021 Jun 10;37(22):4041-4047; PMID: 34110413; doi: 10.1093/bioinformatics/btab434). Commercial humanization services are widely available (e.g. Abzena Limited, Cambridge, UK—www.abzena.com). Similarly, monoclonal antibody engineering, humanization and production protocols are well known (e.g. Antibody Engineering: Methods and Protocols—Nevoltris D and Chames P; New York: Humana Press: Springer, 2018; ISBN: 978-1493986477). Other publications include “Monoclonal Antibodies: A manual of techniques”, H Zola (CRC Press, 1988); “Monoclonal Hybridoma Antibodies: Techniques and Applications”, J G R Hurrell (CRC Press, 1982); Neuberger et al (1988, 8th International Biotechnology Symposium Part 2, 792-799).

A typical whole antibody comprises at least two heavy (H) chains and two light (L) chains interconnected by disulfide bonds. Each heavy chain is comprised of a heavy chain variable region (VH) and a heavy chain constant region (CH). The heavy chain constant region comprises three domains: CH1, CH2 and CH3. The heavy chain constant domains that correspond to the different classes of immunoglobulins are known as α (IgA), δ (IgD), ε (IgE), γ (IgG) and μ (IgM). Each light chain is comprised of a light chain variable region (VL) and a light chain constant region. The light chain constant region comprises just one domain: CL. Light chains are classified as either kappa or lambda. The VH and VL regions can be further subdivided into regions of hypervariability, termed Complementarity Determining Regions (CDR), interspersed with regions that are more conserved, termed framework regions (FW/FWR). Each VH and VL is composed of three CDRs and four FWs (also referred to as FWRs), arranged from amino-terminus to carboxy-terminus in the following order: FWR1, CDR1, FWR2, CDR2, FWR3, CDR3, FWR4. The variable regions of the heavy and light chains contain a binding domain (a paratope) that interacts with an antigen. The constant regions of the antibodies can mediate the binding of the immunoglobulin to host tissues or factors, including various cells of the immune system (such as effector cells) and C1q, which is a component of the complement system.

Sequence identity is as determined using the BLAST program (Tatusova T A et al., FEMS Microbiol Lett. 1999 May 15;174(2):247-50; PMID: 10339815) at the National Center for Biotechnology Information, USA (www.ncbi.nlm.nih.gov) with default parameters.

In certain embodiments, the VH domain additionally comprises at least one of:
(a) FWR1 comprising the amino acid sequence QVILKESGPGMLQPSKTLSLTCSFS (SEQ ID NO: 3) or a sequence having at least 50% sequence identity therewith,
(b) FWR2 comprising the amino acid sequence VSWIRQPSGKSLEWLAA (SEQ ID NO: 5) or a sequence having at least 50% sequence identity therewith,
(c) FWR3 comprising the amino acid sequence YYNPSLKSRLTVSKDTSNTQVFLQITTVDIADTATYYC (SEQ ID NO: 7) or a sequence having at least 50% sequence identity therewith, and
(d) FWR4 comprising the amino acid sequence WGQGVIVXVSS (SEQ ID NO: 9) or a sequence having at least 50% sequence identity therewith.

The VH domain FWRs can also be referred to as “HC-FWR1”, “HC-FWR2”, “HC-FWR3” and “HC-FWR4” as appropriate.

In certain embodiments, the VH domain comprises at least two of the FWR1, FWR2, FWR3 and FWR4. In certain embodiments, it comprises at least 3 of them. In certain embodiments, it comprises all 4 of them.

In certain embodiments, the VL domain additionally comprises at least one of:
(a) FWR1 comprising the amino acid sequence DNQMTQSPPASLSASLGETVTIECRTS (SEQ ID NO: 12) or a sequence having at least 50% sequence identity therewith,
(b) FWR2 comprising the amino acid sequence LAWYQQKPGKSPQLLIY (SEQ ID NO: 14) or a sequence having at least 50% sequence identity therewith,
(c) FWR3 comprising the amino acid sequence RLHIGVPSRFSGSGSGTQFSLKINSLQSEDVASYFC (SEQ ID NO: 16) or a sequence having at least 50% sequence identity therewith, and
(d) FWR4 comprising the amino acid sequence FGAGTKLELN (SEQ ID NO: 18) or a sequence having at least 50% sequence identity therewith.

The VL domain FWRs can also be referred to as “LC-FWR1”, “LC-FWR2”, “LC-FWR3” and “LC-FWR4” as appropriate.

In certain embodiments, the VL domain comprises at least two of the FWR1, FWR2, FWR3 and FWR4. In certain embodiments, it comprises at least 3 of them. In certain embodiments, it comprises all 4 of them.

In certain embodiments, each included FWR domain has at least 60% sequence identity with the corresponding SEQ ID NO. In certain embodiments, each included FWR domain has at least 70% sequence identity with the corresponding SEQ ID NO. In certain embodiments, each included FWR domain has at least 75% sequence identity with the corresponding SEQ. ID NO. In certain embodiments, each included FWR domain has at least 80% sequence identity with the corresponding SEQ ID NO. In certain embodiments, each included FWR domain has at least 85% sequence identity with the corresponding SEQ ID NO. In certain embodiments, each included FWR domain has at least 90% sequence identity with the corresponding SEQ ID NO. In certain embodiments, each included FWR domain has at least 95% sequence identity with the corresponding SEQ ID NO. In certain embodiments, each included FWR has at least 96% sequence identity with the corresponding SEQ ID NO. In certain embodiments, each included FWR has at least 97% sequence identity with the corresponding SEQ ID NO. In certain embodiments, each included FWR has at least 98% sequence identity with the corresponding SEQ ID NO. In certain embodiments, each included FWR has at least 99% sequence identity with the corresponding SEQ ID NO.

In certain embodiments, the FWRs are humanized.

In other aspects of the present invention defining antibodies, the same optional sequence identities apply to the definition of the FWRs.

In certain embodiments, the VH domain has a sequence identity with the sequence of SEQ ID NO: 2 at least that of one of 75%, 80%, 85%, 90%, 95%, 96%, 97%, 98% and 99%. In certain embodiments, the VH domain comprises the sequence of SEQ ID NO: 2, which can be encoded by SEQ ID NO: 1.

TABLE 1
SEQ ID NO: 1
atggacaggcttacttcctcattcctgctgctgcttgtccctgcatatgt
cttgtcccaggttattctgaaagagtctggtcctgggatgttacagccct
ccaagaccctaagcctgacttgctctttttctgggttttcactgagcact
tctggtatggttgtgagctggattcgtcagccttcagggaagagtctcga
gtggctggcagccattgattgggatggtgagaagtactacaacccatctc
tgaaaagcaggctcacagtctccaaggacacctccaacacccaggtattc
ctccagatcaccactgtggacattgcagatactgccacatattactgtgc
tcggagtggtaactactttgagtactggggccaaggagtcatagtcwcag
tctcctcag

TABLE 2
SEQ ID NO: 2
QVILKESGPGMLQPSKTLSLTCSFSGFSLSTSGMVVSWIRQPSGKSLEWL
AAIDWDGEKYYNPSLKSRLTVSKDTSNTQVFLQITTVDIADTATYYCARS
GNYFEYWGQGVIVXVSS

In certain embodiments, the VL domain has a sequence identity with the sequence of SEQ ID NO: 11 at least that of one of 75%, 80%, 85%, 90%, 95%, 96%, 97%, 98% and 99%. In certain embodiments, the VL domain comprises the sequence of SEQ ID NO: 11, which can be encoded by SEQ ID NO: 10.

TABLE 3
SEQ ID NO: 10

atgggtgtccccactcagctcctggggttgttgctactgtggattacag
atgtcatatgtgacaaccagatgacacagtctccagcttccctgtctgc
atctctgggagaaactgtcaccatcgaatgtcgaacaagcgaggacatt
tacaatgctttagcatggtatcagcagaagccagggaaatctcctcagc
tcctgatctatgatgcaaataggttgcatattggggtcccatcacggtt
cagtggcagtggatctggtacacagttttctctcaagataaacagcctg
caatctgaagatgtcgcaagttatttctgtcaacagtattacgattatc
cgaacacgtttggagctgggaccaagctggagctgaacc

TABLE 4
SEQ ID NO: 11

DNQMTQSPASLSASLGETVTIECRTSEDIYNALAWYQQKPGKSPQLLIY
DANRLHIGVPSRFSGSGSGTQFSLKINSLQSEDVASYFCQQYYDYPNTF
GAGTKLELN

As detailed below, these sequences are from the rat 3F6 monoclonal antibody raised against human IL-11 and which displays excellent binding characteristics for human IL-11.

In certain embodiments, the antibody comprises the VH domain of SEQ ID NO: 2 and the VL domain of SEQ ID NO: 11. An exemplary antibody including those domains is CSJ-B-s-3F6 (details below).

TABLE 5
CDR and FWR sequences of 3F6 HC (Heavy Chain) and LC (Light Chain):

| | SEQ ID | | SEQ ID |
|---|---|---|---|
| HC | NO | LC | NO |
| FWR1: QVILKESGPG MLQPSKTLSL | 3 | FWR1: DNQMTQSPAS | 12 |
| TCSES | | LSASLGETVT IECRTS | |
| CDR1: GFSLSTSGMV | 4 | CDR1: EDIYNA | 13 |
| FWR2: VSWIRQPSGK SLEWLAA | 5 | FWR2: LAWYQQKPGK SPQLLIY | 14 |
| CDR2: IDWDGEK | 6 | CDR2: DAN | — |
| FWR3: YYNPSLKSRL TVSKDTSNTQ | 7 | FWR3: RLHIGVPSRF | 16 |
| VFLQITTVDI ADTATYYC | | SGSGSGTQFS LKINSLQSED | |
| | | VASYFC | |
| CDR3: ARSGNYFEY | 8 | CDR3: QQYYDYPNT | 17 |
| FWR4: WGQGVIVXVS S | 9 | FWR4: FGAGTKLELN | 18 |

The 3F6 light chain CDR2 sequence is 3 amino acids in length and as such cannot be represented in accordance with WIPO standard ST.26 (which sets a minimum amino acid sequence length of 4). For the avoidance of doubt, any reference to SEQ ID NO: 15 is reference to the amino acid sequence DAN.

The full VH and VL sequences are given in SEQ ID NOs: 1, 2, 10 and 11. The WIPO ST.26 formatted nucleotide sequences (SEQ ID NOs: 1 and 10) detail the location and identity of domains (FWRs, CDRs).

Unless the context dictates otherwise, the various optional features (e.g. “In certain embodiments . . . ”) disclosed in relation to this aspect are equally applicable to other aspects.

### Humanized 3F6

A number of humanized 3F6 variants have been produced based on the 3F6 mAb sequences. These humanized variants (detailed below) are VH0/VL0, CVH3 (D54E)/CVL3, CVH3 (D54E)/CVL3+YTE, CVH4 (D54E)/CVL3, CVH4 (D54E)/CVL3+YTE, GVH5 (D54E)/CVL3, GVH5 (D54E)/CVL3+YTE, GVHS (D54E)/GVL5, and GVH5 (D54E)/GVL5+YTE.

The base construct is the VH0/VL0 mAb (SEQ ID NOs: 91 and 92—the VH0 and VL0 heavy and light chains respectively) which is a chimeric antibody comprising (a) the 3F6 VH and VL CDRs and FWRs (i.e. the 3F6 heavy and light chain variable domains), with the ‘X’ residue in the 3F6 VH FWR4 (SEQ ID NO: 9) being a T (Threonine), and (b) human heavy and light chain constant regions (i.e. human heavy and light chain constant domains).

TABLE 6
SEQ ID NO: 91

QVILKESGPGMLQPSKTLSLTCSFSGFSLSTSGMVVSWIRQPSGKSLEW
LAAIDWDGEKYYNPSLKSRLTVSKDTSNTQVFLQITTVDIADTATYYCA
RSGNYFEYWGQGVIVTVSSASTKGPSVFPLAPSSKSTSGGTAALGCLVK
DYFPEPVTVSWNSGALTSGVHTFPAVLQSSGLYSLSSVVTVPSSSLGTQ
TYICNVNHKPSNTKVDKRVEPKSCDKTHTCPPCPAPELLGGPSVFLFPP
KPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSL
SLSPGK

TABLE 7
SEQ ID NO: 92

DNQMTQSPASLSASLGETVTIECRTSEDIYNALAWYQQKPGKSPQLLIY
DANRLHIGVPSRFSGSGSGTQFSLKINSLQSEDVASYFCQQYYDYPNTF
GAGTKLELNRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

The mAb heavy chains have been further humanized with modifications in the VH FWR1, VH FWR2, VH CDR2, VH FWR3 and VH FWR4 domains. The mAb light chains have been further humanized with modifications in the VL FWR1, VL FWR2, VL FWR3 and VL FWR4 domains.

The heavy chain of the mAbs comprises the VH domain, together with a standard human IGHG1 (immunoglobulin heavy constant gamma 1), i.e. CH1-3 domain.

The light chain of the mAbs comprises the VL domain, together with a standard human IGKC (immunoglobulin kappa constant), i.e. CL domain.

The heavy chain has also been modified in various of the mAbs with the introduction of a YTE triple mutation in the Fc region (Robbie G J et al., Antimicrob Agents Chemother. 2013 December; 57(12):6147-53.

PMID: 24080653; doi: 10.1128/AAC.01285-13) to extend half-life (i.e. extend elimination half-life) in humans. Thus, in certain embodiments, the antibody comprises a human IgG1 heavy chain constant region with amino acids Y135, T137, and E139 (with a 117 amino acid VH domain, this corresponds to Y252, T254 and E256 in a full IgG1 heavy chain). In certain embodiments, the YTE triplet mutation in the heavy chain constant region is M135Y, S137T, and T139E (i.e. M252Y, S254T, and T256E in the full heavy chain sequence).

To summarize, the humanized mAbs are as follows (SEQ ID NOs: 91-100 and 124 are the full heavy and light chain sequences; see FIG. 7).

TABLE 8

| | | +YTE | Heavy chain | | Light chain |
|---|---|---|---|---|---|
| Name | VH | (VH Fc) | SEQ ID NO | VL | SEQ ID NO |
| mAb VH0/VL0 | VH0 | No | 91 | VL0 | 92 |
| mAb CVH3 (D54E)/CVL3 | CVH3 (D54E) | No | 93 | CVL3 | 94 |
| mAb CVH4 (D54E)/CVL3 | CVH4 (D54E) | No | 95 | CVL3 | 94 |
| mAb GVH5 (D54E)/CVL3 | GVH5 (D54E) | No | 96 | CVL3 | 94 |
| mAb GVH5 (D54E)/GVL5 | GVH5 (D54E) | No | 96 | GVL5 | 97 |
| mAb CVH3 (D54E)/CVL3 + YTE | CVH3 (D54E) | Yes | 98 | CVL3 | 94 |
| mAb CVH4 (D54E)/CVL3 + YTE | CVH4 (D54E) | Yes | 99 | CVL3 | 94 |
| mAb GVH5 (D54E)/CVL3 + YTE | GVH5 (D54E) | Yes | 100 | CVL3 | 94 |
| mAb GVH5 (D54E)/GVL5 + YTE | GVH5 (D54E) | Yes | 124 | GVL5 | 97 |

Thus, in certain embodiments an antibody according to the present invention comprises a heavy chain having the sequence of SEQ ID NO: 91 and a light chain having the sequence of SEQ ID NO: 92 (i.e. mAb VH0/VL0).

In certain embodiments an antibody according to the present invention comprises a heavy chain having the sequence of SEQ ID NO: 93 and a light chain having the sequence of SEQ ID NO: 94 (i.e. mAb CVH3 (D54E)/CVL3).

In certain embodiments an antibody according to the present invention comprises a heavy chain having the sequence of SEQ ID NO: 95 and a light chain having the sequence of SEQ ID NO: 94 (i.e. mAb CVH4 (D54E)/CVL3).

In certain embodiments an antibody according to the present invention comprises a heavy chain having the sequence of SEQ ID NO: 96 and a light chain having the sequence of SEQ ID NO: 94 (i.e. mAb GVH5 (D54E)/CVL3) In certain embodiments an antibody according to the present invention comprises a heavy chain having the sequence of SEQ ID NO: 96 and a light chain having the sequence of SEQ ID NO: 97 (i.e. mAb GVH5 (D54E)/GVLS).

In certain embodiments an antibody according to the present invention comprises a heavy chain having the sequence of SEQ ID NO: 98 and a light chain having the sequence of SEQ ID NO: 94 (i.e. mAb CVH3 (D54E)/CVL3+YTE).

In certain embodiments an antibody according to the present invention comprises a heavy chain having the sequence of SEQ ID NO: 99 and a light chain having the sequence of SEQ ID NO: 94 (i.e. mAb CVH4 (D54E)/CVL3+YTE).

In certain embodiments an antibody according to the present invention comprises a heavy chain having the sequence of SEQ ID NO: 100 and a light chain having the sequence of SEQ ID NO: 94 (i.e. mAb GVH5 (D54E)/CVL3+YTE).

In certain embodiments an antibody according to the present invention comprises a heavy chain having the sequence of SEQ ID NO: 124 and a light chain having the sequence of SEQ ID NO: 97 (i.e. mAb GVH5 (D54E)/GVL5+YTE).

The VH and VL domain sequences of those full heavy and light chain sequences are as follows (SEQ ID NOs: 116-122; FIG. 8).

TABLE 9

| Name | VH | SEQ ID NO | VL | SEQ ID NO |
|---|---|---|---|---|
| mAb VH0/VL0 | VH0 | 116 | VL0 | 117 |
| mAb CVH3 (D54E)/CVL3 | CVH3 (D54E) | 118 | CVL3 | 119 |
| mAb CVH4 (D54E)/CVL3 | CVH4 (D54E) | 120 | CVL3 | 119 |
| mAb GVH5 (D54E)/CVL3 | GVH5 (D54E) | 121 | CVL3 | 119 |
| mAb GVH5 (D54E)/GVL5 | GVH5 (D54E) | 121 | GVL5 | 122 |

In terms of overall sequence identity of the VH and VL domains of the antibody with those of the 3F6 mAb, in certain embodiments, the antibody VH domain has at least 60% sequence identity with SEQ ID NO: 2 (the 3F6 VH amino acid sequence). In certain embodiments, it has at least 65% sequence identity. In certain embodiments, it has at least 70% sequence identity. In certain embodiments, it has at least 75% sequence identity. In certain embodiments, it has at least 80% sequence identity. In certain embodiments, it has at least 85% sequence identity. In certain embodiments, it has at least 90% sequence identity.

In the above sequences, CVH3 (D54E) has 83% sequence identity with the 3F6 VH. CVH4 has 81% sequence identity with the 3F6 VH. GVH5 has 75% sequence identity with the 3F6 VH.

In certain embodiments, the antibody VL domain has at least 60% sequence identity with SEQ ID NO: 11 (the 3F6 VL amino acid sequence). In certain embodiments, it has at least 65% sequence identity.

In certain embodiments, it has at least 70% sequence identity. In certain embodiments, it has at least 75% sequence identity. In certain embodiments, it has at least 80% sequence identity. In certain embodiments, it has at least 85% sequence identity. In certain embodiments, it has at least 90% sequence identity.

In the above sequences, CVL3 has 84% sequence identity with the 3F6 VL. GVL5 has 83% sequence identity with the 31F6 VL.

With regard to CDRs and FWRs in the VH sequences:

TABLE 10

| | | SEQ |
|---|---|---|
| mAb Name | VH FWR1 | ID NO |
| mAb 3F6 | QVILKESGPG MLQPSKTLSL TCSFS | 3 |
| (non-humanized) | | |
| mAb VHO/VLO | QVILKESGPG MLQPSKTLSL TCSES | 3 |
| (non-humanized) | | |
| mAb CVH3 (D54E)/ CVL3 | 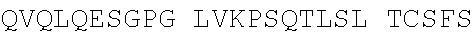 | 101 |
| mAb CVH4 (D54E)/ CVL3 | 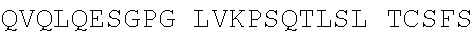 | 101 |
| mAb GVH5 (D54E)/ CVL3 | 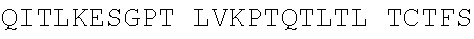 | 102 |
| mAb GVH5 (D54E)/ GVL5 | 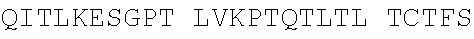 | 102 |
| mAb CVH3 (D54E)/ CVL3 + YTE | 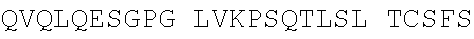 | 101 |
| mAb CVH4 (D54E)/ CVL3 + YTE | 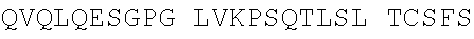 | 101 |
| mAb GVH5 (D54E)/ CVL3 + YTE | 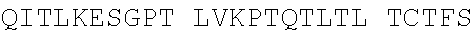 | 102 |

TABLE 11

| mAb Name | VH CDR1 | SEQ ID NO |
|---|---|---|
| mAb 3F6 (non-humanized) | GFSLSTSGMV | 4 |
| mAb VH0/VL0 (non-humanized) | GFSLSTSGMV | 4 |
| mAb CVH3 (D54E)/CVL3 | GFSLSTSGMV | 4 |
| mAb CVH4 (D54E)/CVL3 | GFSLSTSGMV | 4 |
| mAb GVH5 (D54E)/CVL3 | GFSLSTSGMV | 4 |
| mAb GVH5 (D54E)/GVL5 | GFSLSTSGMV | 4 |
| mAb CVH3 (D54E)/CVL3 + YTE | GFSLSTSGMV | 4 |
| mAb CVH4 (D54E)/CVL3 + YTE | GFSLSTSGMV | 4 |
| mAb GVH5 (D54E)/CVL3 + YTE | GFSLSTSGMV | 4 |

TABLE 12

| mAb Name | VH FWR2 | SEQ ID NO |
|---|---|---|
| mAb 3F6 | VSWIRQPSGK SLEWLAA | 5 |
| (non-humanized) | | |
| mAb VH0/VLO | VSWIRQPSGK SLEWLAA | 5 |
| (non-humanized) | | |
| mAb CVH3 (D54E)/CVL3 | 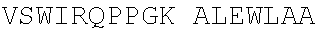 | 103 |
| mAb CVH4 (D54E)/CVL3 | 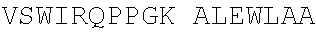 | 103 |
| mAb GVH5 (D54E)/CVL3 | 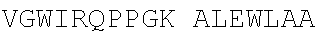 | 104 |
| mAb GVH5 (D54E)/GVL5 | 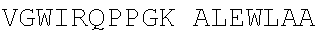 | 104 |
| mAb CVH3 (D54E)/ CVL3 + YTE | 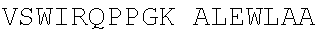 | 103 |
| mAb CVH4 (D54E)/ CVL3 + YTE | 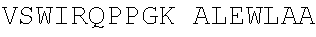 | 103 |
| mAb GVH5 (D54E)/ CVL3 + YTE | 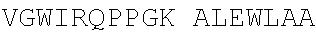 | 104 |

TABLE 13

| mAb Name | VH CDR2 | SEQ ID NO |
|---|---|---|
| mAb 3F6 (non-humanized) | IDWDGEK | 6 |
| mAb VH0/VLO (non-humanized) | IDWDGEK | 6 |
| mAb CVH3 (D54E)/CVL3 | 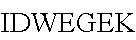 | 105 |
| mAb CVH4 (D54E)/CVL3 | 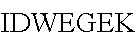 | 105 |
| mAb GVH5 (D54E)/CVL3 | 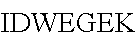 | 105 |
| mAb GVH5 (D54E)/GVL5 | 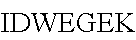 | 105 |
| mAb CVH3 (D54E)/CVL3 + YTE | 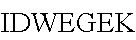 | 105 |
| mAb CVH4 (D54E)/CVL3 + YTE | 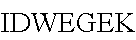 | 105 |
| mAb GVH5 (D54E)/CVL3 + YTE | 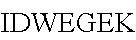 | 105 |

TABLE 14

| | | SEQ |
|---|---|---|
| mAb Name | VH FWR3 | ID NO |
| mAb 3F6 (non- | YYNPSLKSRL TVSKDTSNTQ VFLQITTVDI ADTATYYC | 7 |
| humanized) | | |
| mAb VHO/VLO | YYNPSLKSRL TVSKDTSNTQ VFLQITTVDI ADTATYYC | 7 |
| (non-humanized) | | |
| mAb CVH3 (D54E)/CVL3 | 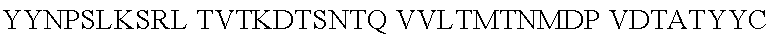 | 106 |
| mAb CVH4 (D54E)/CVL3 | 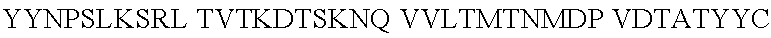 | 107 |
| mAb GVH5 (D54E)/CVL3 | 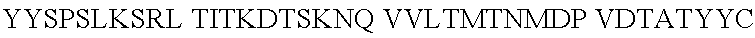 | 108 |
| mAb GVH5 | | |
| (D54E)/GVL5 | | |
| mAb CVH3(D54E)/ CVL3 + YTE | 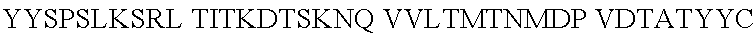 | 106 |
| mAb CVH4(D54E)/ CVL3 + YTE | 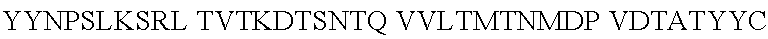 | 107 |
| mAb GVH5(D54E)/ CVL3 + YTE | 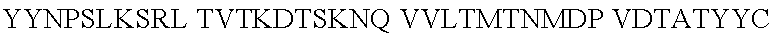 | 108 |

TABLE 15

| mAb Name | VH CDR3 | SEQ ID NO |
|---|---|---|
| mAb 3F6 (non-humanized) | ARSGNYFEY | 8 |
| mAb VH0/VL0 (non-humanized) | ARSGNYFEY | 8 |
| mAb CVH3 (D54E)/CVL3 | ARSGNYFEY | 8 |
| mAb CVH4 (D54E)/CVL3 | ARSGNYFEY | 8 |
| mAb GVH5 (D54E)/CVL3 | ARSGNYFEY | 8 |
| mAb GVH5 (D54E)/GVL5 | ARSGNYFEY | 8 |
| mAb CVH3 (D54E)/CVL3 + YTE | ARSGNYFEY | 8 |
| mAb CVH4 (D54E)/CVL3 + YTE | ARSGNYFEY | 8 |
| mAb GVH5 (D54E)/CVL3 + YTE | ARSGNYFEY | 8 |

TABLE 16

| mAb Name | VH FWR4 | SEQ ID NO |
|---|---|---|
| mAb 3F6 (non-humanized) | WGQGVIVXVS | 9 |
| mAb VHO/VLO (non-humanized) | 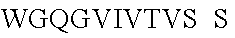 | 109 |
| mAb CVH3 (D54E)/CVL3 | 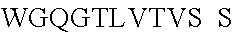 | 109 |
| mAb CVH4 (D54E)/CVL3 | 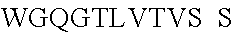 | 109 |
| mAb GVH5 (D54E)/CVL3 | 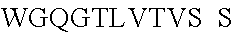 | 109 |
| mAb GVH5 (D54E)/GVL5 | 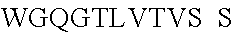 | 109 |
| mAb CVH3 (D54E)/CVL3 + YTE | 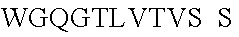 | 109 |
| mAb CVH4 (D54E)/CVL3 + YTE | 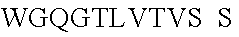 | 109 |
| mAb GVH5 (D54E)/CVL3 + YTE | 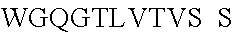 | 109 |

TABLE 17

| mAb Name | VL FWR1 | SEQ ID NO |
|---|---|---|
| mAb 3F6 (non-humanized) | DNOMTQSPAS LSASLGETVT IECRTS | 12 |
| mAb VH0/VLO (non-humanized) | DNOMTQSPAS LSASLGETVT IECRTS | 12 |
| mAb CVH3 (D54E)/CVL3 | 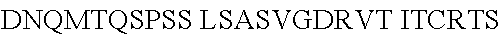 | 110 |
| mAb CVH4 (D54E)/CVL3 | 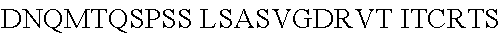 | 110 |
| mAb GVH5 (D54E)/CVL3 | 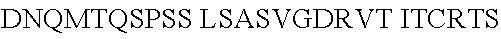 | 110 |
| mAb GVH5 (D54E)/GVL5 | 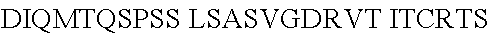 | 111 |
| mAb CVH3 (D54E)/CVL3 + YTE | 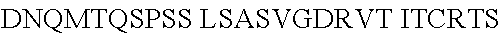 | 110 |
| mAb CVH4 (D54E)/CVL3 + YTE | 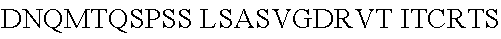 | 110 |
| mAb GVH5 (D54E)/CVL3 + YTE | 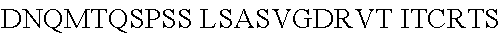 | 110 |

TABLE 18

| mAb Name | VL CDR1 | SEQ ID NO |
|---|---|---|
| mAb 3F6 (non-humanized) | EDIYNA | 13 |
| mAb VH0/VL0 (non-humanized) | EDIYNA | 13 |
| mAb CVH3 (D54E)/CVL3 | EDIYNA | 13 |
| mAb CVH4 (D54E)/CVL3 | EDIYNA | 13 |
| mAb GVH5 (D54E)/CVL3 | EDIYNA | 13 |
| mAb GVH5 (D54E)/GVL5 | EDIYNA | 13 |
| mAb CVH3 (D54E)/CVL3 + YTE | EDIYNA | 13 |
| mAb CVH4 (D54E)/CVL3 + YTE | EDIYNA | 13 |
| mAb GVH5 (D54E)/CVL3 + YTE | EDIYNA | 13 |

TABLE 19

| | | SEQ |
|---|---|---|
| mAb Name | VL FWR2 | ID NO |
| mAb 3F6 (non-humanized) | LAWYQQKPGK SPQLLIY | 14 |
| mAb VHO/VLO | LAWYQQKPGK SPOLLIY | 14 |
| (non-humanized) | | |
| mAb CVH3 (D54E)/CVL3 | 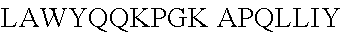 | 112 |
| mAb CVH4 (D54E)/CVL3 | 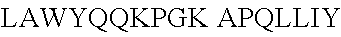 | 112 |
| mAb GVH5 (D54E)/CVL3 | 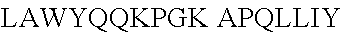 | 112 |
| mAb GVH5 (D54E)/GVL5 | 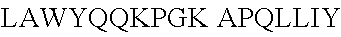 | 112 |
| mAb CVH3 (D54E)/ CVL3 + YTE | 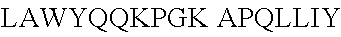 | 112 |
| mAb CVH4 (D54E)/ CVL3 + YTE | 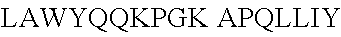 | 112 |
| mAb GVH5 (D54E)/ CVL3 + YTE | 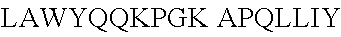 | 112 |

TABLE 20

| mAb Name | VL CDR2 | SEQ ID NO |
|---|---|---|
| mAb 3F6 (non-humanized) | DAN | |
| mAb VH0/VL0 (non-humanized) | DAN | |
| mAb CVH3 (D54E)/CVL3 | DAN | |
| mAb CVH4 (D54E)/CVL3 | DAN | |
| mAb GVH5 (D54E)/CVL3 | DAN | |
| mAb GVH5 (D54E)/GVL5 | DAN | |
| mAb CVH3 (D54E)/CVL3 + YTE | DAN | |
| mAb CVH4 (D54E)/CVL3 + YTE | DAN | |
| mAb GVH5 (D54E)/CVL3 + YTE | DAN | |

TABLE 21

| mAb Name | VL FWR3 | SEQ ID NO |
|---|---|---|
| mAb 3F6 (non- | RLHIGVPSRF SGSGSGTOFS LKINSLOSED VASYFC | 16 |
| humanized) | | |
| mAb VHO/VLO | RLHIGVPSRF SGSGSGTOFS LKINSLQSED VASYFC | 16 |
| (non-humanized) | | |
| mAb CVH3 | 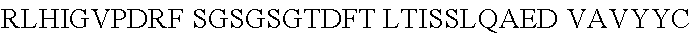 | 113 |
| (D54E)/CVL3 | | |
| mAb CVH4 | 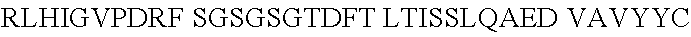 | 113 |
| (D54E)/CVL3 | | |
| mAb GVH5 | 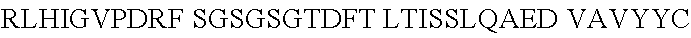 | 113 |
| (D54E)/CVL3 | | |
| mAb GVH5 | 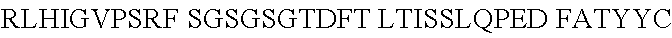 | 114 |
| (D54E)/GVL5 | | |
| mAb CVH3 | 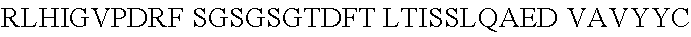 | 113 |
| (D54E)/CVL3 + YTE | | |
| mAb CVH4 | 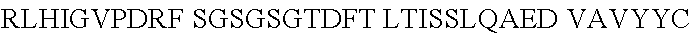 | 113 |
| (D54E)/CVL3 + YTE | | |
| mAb GVH5 | 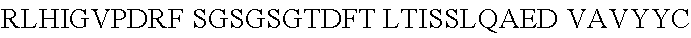 | 113 |
| (D54E)/CVL3 + YTE | | |

TABLE 22

| mAb Name | VL CDR3 | SEQ ID NO |
|---|---|---|
| mAb 3F6 (non-humanized) | QQYYDYPNT | 17 |
| mAb VH0/VL0 (non-humanized) | QQYYDYPNT | 17 |
| mAb CVH3 (D54E)/CVL3 | QQYYDYPNT | 17 |
| mAb CVH4 (D54E)/CVL3 | QQYYDYPNT | 17 |
| mAb GVH5 (D54E)/CVL3 | QQYYDYPNT | 17 |
| mAb GVH5 (D54E)/GVL5 | QQYYDYPNT | 17 |
| mAb CVH3 (D54E)/CVL3 + YTE | QQYYDYPNT | 17 |
| mAb CVH4 (D54E)/CVL3 + YTE | QQYYDYPNT | 17 |
| mAb GVH5 (D54E)/CVL3 + YTE | QQYYDYPNT | 17 |

TABLE 23

| mAb Name | VL FWR4 | SEQ ID NO |
|---|---|---|
| mAb 3F6 (non-humanized) | FGAGTKLELN | 18 |
| mAb VHO/VLO (non-humanized) | FGAGTKLELN | 18 |
| mAb CVH3 (D54E)/CVL3 | 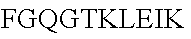 | 115 |
| mAb CVH4 (D54E)/CVL3 | 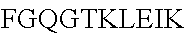 | 115 |
| mAb GVH5 (D54E)/CVL3 | 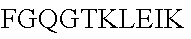 | 115 |
| mAb GVH5 (D54E)/GVL5 | 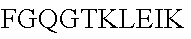 | 115 |
| mAb CVH3 (D54E)/CVL3 + YTE | 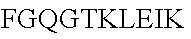 | 115 |
| mAb CVH4 (D54E)/CVL3 + YTE | 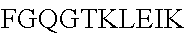 | 115 |
| mAb GVH5 (D54E)/CVL3 + YTE | 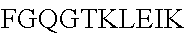 | 115 |

Thus, in certain embodiments, the antibody has a VH CDR2 of SEQ ID NO: 105.

In certain embodiments, the antibody has a VH FWR1 of SEQ ID NO: 101. In other embodiments, it has a VH FWR1 of SEQ ID NO: 102.

In certain embodiments, the antibody has a VH FWR2 of SEQ ID NO: 103. In other embodiments, it has a VH FWR2 of SEQ ID NO: 104.

In certain embodiments, the antibody has a VH FWR3 of SEQ ID NO: 106. In other embodiments, it has a VH FWR3 of SEQ ID NO: 107. In other embodiments, it has a VH FWR3 of SEQ ID NO: 108.

In certain embodiments, the antibody has a VH FWR4 of SEQ ID NO: 109.

In certain embodiments, the antibody has a VL FWR1 of SEQ ID NO: 110. In other embodiments, it has a VL FWR1 of SEQ ID NO: 111.

In certain embodiments, the antibody has a VL FWR2 of SEQ ID NO: 112.

In certain embodiments, the antibody has a VL FWR3 of SEQ ID NO: 113. In other embodiments, it has a VL FWR3 of SEQ ID NO: 114. 9B9: According to a second aspect of the present invention there is provided an antibody that binds to human IL-11, the antibody comprising a heavy chain (HC) variable region sequence and a light chain (LC) variable region sequence,
(i) the heavy chain variable region sequence comprising the CDR3 and at least one additional CDR selected from the group consisting of:
   (a) HC-CDR1 comprising the amino acid sequence GYTFTSYV (SEQ ID NO: 22) or a variant having one, two or three amino acid substitutions therein,
   (b) HC-CDR2 comprising the amino acid sequence IYPYNDDT (SEQ ID NO: 24) or a variant having one or two amino acid substitutions therein, and
   (c) HC-CDR3 comprising the amino acid sequence TRERATYGDLAWFAY (SEQ ID NO: 26) or a variant having one or two amino acid substitutions therein; and
(ii) the light chain variable region sequence comprising the CDR3 and at least one additional CDR selected from the group consisting of:
   (a) LC-CDR1 comprising the amino acid sequence ENIDSYGNSF (SEQ ID NO: 31) or a variant having one or two amino acid substitutions therein,
   (b) LC-CDR2 comprising the amino acid sequence LAS or a variant having one amino acid substitution therein, and
   (c) LC-CDR3 comprising the amino acid sequence QQNNEDPYT (SEQ ID NO: 35) or a variant having one or two amino acid substitutions therein.

In certain embodiments, the VH domain comprises all three of the CDR1, CDR2 and CDR3. In certain embodiments, the VL domain comprises all three of the CDR1, CDR2 and CDR3. In certain embodiments, the VH domain comprises all three of the CDR1, CDR2 and CDR3, and the VL domain comprises all three of the CDR1, CDR2 and CDR3.

In certain embodiments, the HC-CDR1 has one amino acid substitution. In certain embodiments, it has no substitutions.

In certain embodiments, the HC-CDR2 has one amino acid substitution. In certain embodiments, it has no substitutions.

In certain embodiments, the HC-CDR3 has one amino acid substitution. In certain embodiments, it has no substitutions.

In certain embodiments, the LC-CDR1 has one amino acid substitution. In certain embodiments, it has no substitutions.

In certain embodiments, the LC-CDR2 has one amino acid substitution. In certain embodiments, it has no substitutions.

In certain embodiments, the LC-CDR3 has one amino acid substitution. In certain embodiments, it has no substitutions.

In certain embodiments, the VH domain additionally comprises at least one of:
(a) FWR1 comprising the amino acid sequence EVHLQQSGPELVKPGASVKMSCKAS (SEQ ID NO: 21) or a sequence having at least 50% sequence identity therewith,
(b) FWR2 comprising the amino acid sequence IHWVKQRPGQGLEWIGN (SEQ ID NO: 23) or a sequence having at least 50% sequence identity therewith,
(c) FWR3 comprising the amino acid sequence NYNEKFKGKATLTSDKSSSTAYMELSSLTSEESAVYFC (SEQ ID NO: 25) or a sequence having at least 50% sequence identity therewith, and
(d) FWR4 comprising the amino acid sequence WGQGTLVTVSA (SEQ ID NO: 27) or a sequence having at least 50% sequence identity therewith.

In certain embodiments, the VH domain comprises at least two of the FWR1, FWR2, FWR3 and FWR4.

In certain embodiments, it comprises at least 3 of them. In certain embodiments, it comprises all 4 of them.

In certain embodiments, the VL domain additionally comprises at least one of:
(a) FWR1 comprising the amino acid sequence NIVLTQSPASLAVSLGQRATISCRAS (SEQ ID NO: 30) or a sequence having at least 50% sequence identity therewith,
(b) FWR2 comprising the amino acid sequence MHWYQQKPGQPPKLLIY (SEQ ID NO: 32) or a sequence having at least 50% sequence identity therewith,
(c) FWR3 comprising the amino acid sequence ILESGVPARFSGSGSRTDFTLTIDPVEADDAATYYC (SEQ ID NO: 34) or a sequence having at least 50% sequence identity therewith, and
(d) FWR4 comprising the amino acid sequence FGGGTKLEIK (SEQ ID NO: 36) or a sequence having at least 50% sequence identity therewith.

In certain embodiments, the VL domain comprises at least two of the FWR1, FWR2, FWR3 and FWR4. In certain embodiments, it comprises at least 3 of them. In certain embodiments, it comprises all 4 of them.

In certain embodiments, each included FWR has at least 85% sequence identity with the corresponding SEQ ID NO. In certain embodiments, each included FWR has at least 90% sequence identity with the corresponding SEQ ID NO. In certain embodiments, each included FWR has at least 95% sequence identity with the corresponding SEQ ID NO. In certain embodiments, each included FWR has at least 96% sequence identity with the corresponding SEQ ID NO. In certain embodiments, each included FWR has at least 97% sequence identity with the corresponding SEQ ID NO. In certain embodiments, each included FWR has at least 98% sequence identity with the corresponding SEQ ID NO. In certain embodiments, each included FWR has at least 99% sequence identity with the corresponding SEQ ID NO.

In certain embodiments, the FWRs are humanized.

In certain embodiments, the VH domain has a sequence identity with the sequence of SEQ ID NO: 20 at least that of one of 75%, 80%, 85%, 90%, 95%, 96%, 97%, 98% and 99%. In certain embodiments, the VH domain comprises the sequence of SEQ ID NO: 20, which can be encoded by SEQ ID NO: 19.

TABLE 24
SEQ ID NO: 19

atggaatggagctggatatttctctttctcctgtcaggaactgcaggtg
tccactctgaggtccacctgcagcagtctggacctgaactggtaaagcc
tggggcttcagtgaagatgtcctgcaaggcttctggatacacattcact
agctatgttatacactgggtgaagcagaggcctgggcagggccttgagt
ggattggaaatatttatccttacaatgatgatactaactacaatgagaa
gttcaaaggcaaggccacactgacttcagacaaatcctccagcacagcc
tacatggagctcagcagcctgacctctgaggagtctgcggtctatttct
gtacaagagagagggctacctatggtgaccttgcctggtttgcttactg
gggccaagggactctggtcactgtctctgcag

TABLE 25
SEQ ID NO: 20

EVHLQQSGPELVKPGASVKMSCKASGYTFTSYVIHWVKQRPGQGLEWIG
NIYPYNDDTNYNEKFKGKATLTSDKSSSTAYMELSSLTSEESAVYFCTR
ERATYGDLAWFAYWGQGTLVTVSA

In certain embodiments, the VL domain has a sequence identity with the sequence of SEQ ID NO: 29 at least that of one of 75%, 80%, 85%, 90%, 95%, 96%, 97%, 98% and 99%. In certain embodiments, the VL domain comprises the sequence of SEQ ID NO: 29, which can be encoded by SEQ ID NO: 28.

TABLE 26
SEQ ID NO: 28

atggagacagacacactcctgctatgggtgctgctgctctgggttccag
gttccacaggtaacattgtgctgacccaatctccagcttctttggctgt
gtctctagggcagagggccaccatatcctgcagagccagtgaaaatatt
gatagttatggcaatagttttatgcactggtaccagcagaaaccaggac
agccacccaaactcctcatctatcttgcatccatcctagaatctggggt
ccctgccaggttcagtggcagtgggtctaggacagacttcaccctcacc
attgatcctgtggaggctgatgatgctgcaacctattactgtcagcaaa
ataatgaggatccgtacacgttcggaggggggaccaagctggaaataaa
ac

TABLE 27
SEQ ID NO: 29

NIVLTQSPASLAVSLGQRATISCRASENIDSYGNSFMHWYQQKPGQPPK
LLIYLASILESGVPARFSGSGSRTDFTLTIDPVEADDAATYYCQQNNED
PYTFGGGTKLEIK

As detailed below, these sequences are from the mouse 9B9 monoclonal antibody raised against human IL-11 and which displays excellent binding characteristics.

In certain embodiments, the antibody comprises the VH domain of SEQ ID NO: 20 and the VL domain of SEQ ID NO: 29. An exemplary antibody including those domains is CSI-s-9B9 (details below).

TABLE 28
CDR and FWR sequences of 9B9 HC (Heavy Chain) and LC (Light Chain):

| | SEQ ID | | SEQ ID |
|---|---|---|---|
| HC | NO | LC | NO |
| FWR1: EVHLQQSGPE | 21 | FWR1: NIVLTQSPAS | 30 |
| LVKPGASVKM SCKAS | | LAVSLGQRAT ISCRAS | |
| CDR1: GYTFTSYV | 22 | CDR1: ENIDSYGNSF | 31 |
| FWR2: IHWVKQRPGQ GLEWIGN | 23 | FWR2: MHWYQQKPGQ | 32 |
| CDR2: IYPYNDDT | 24 | CDR2: LAS | — |
| FWR3: NYNEKEKGKA | 25 | FWR3: ILESGVPARF | 34 |
| TLTSDKSSST AYMELSSLTS | | SGSGSRTDFT LTIDPVEADD | |
| EESAVYFC | | AATYYC | |
| CDR3: TRERATYGDL AWFAY | 26 | CDR3: QQNNEDPYT | 35 |
| FWR4: WGQGTLVTVS A | 27 | FWR4: FGGGTKLEIK | 36 |

The 9B9 light chain CDR2 sequence is 3 amino acids in length and as such cannot be represented in accordance with WIPO standard ST.26 (which sets a minimum amino acid sequence length of 4). For the avoidance of doubt, any reference to SEQ ID NO: 33 is reference to the amino acid sequence LAS.

The full VH and VL sequences are given in SEQ ID NOs: 19, 20, 28 and 29. The WIPO ST.26 formatted nucleotide sequences detail the location and identity of domains (FWRs, CDRs) within them. 2B11:

According to a third aspect of the present invention there is provided an antibody that binds to human IL-11, the antibody comprising a heavy chain (HC) variable region sequence and a light chain (LC) variable region sequence,
(i) the heavy chain variable region sequence comprising the CDR3 and at least one additional CDR selected from the group consisting of:
   (a) HC-CDR1 comprising the amino acid sequence GFSLDNYN (SEQ ID NO: 40) or a variant having one, two or three amino acid substitutions therein,
   (b) HC-CDR2 comprising the amino acid sequence IWTDGIT (SEQ ID NO: 42) or a variant having one or two amino acid substitutions therein, and
   (c) HC-CDR3 comprising the amino acid sequence ARSYYYSLYYFDY (SEQ ID NO: 44) or a variant having one or two amino acid substitutions therein; and
(ii) the light chain variable region sequence comprising the CDR3 and at least one additional CDR selected from the group consisting of:
   (a) LC-CDR1 comprising the amino acid sequence EDIYSN (SEQ ID NO: 49) or a variant having one or two amino acid substitutions therein,
   (b) LC-CDR2 comprising the amino acid sequence DAH or a variant having one amino acid substitution therein, and
   (c) LC-CDR3 comprising the amino acid sequence QQYDNYNT (SEQ ID NO: 53) or a variant having one or two amino acid substitutions therein.

In certain embodiments, the VH domain comprises all three of the CDR1, CDR2 and CDR3. In certain embodiments, the VL domain comprises all three of the CDR1, CDR2 and CDR3. In certain embodiments, the VH domain comprises all three of the CDR1, CDR2 and CDR3, and the VL domain comprises all three of the CDR1, CDR2 and CDR3.

In certain embodiments, the VH domain additionally comprises at least one of:
(a) FWR1 comprising the amino acid sequence QVQLKESGPGLVQPSQTLSLTCTVS (SEQ ID NO: 39) or a sequence having at least 50% sequence identity therewith,
(b) FWR2 comprising the amino acid sequence VHWVRQPTGGGLEWLGG (SEQ ID NO: 41) or a sequence having at least 50% sequence identity therewith,
(c) FWR3 comprising the amino acid sequence DYTSPLRSRLSISRDTSKSQVFLKMKSLQTEDIATYYC (SEQ ID NO: 43) or a sequence having at least 50% sequence identity therewith, and
(d) FWR4 comprising the amino acid sequence WGQGVMVTVSS (SEQ ID NO: 45) or a sequence having at least 50% sequence identity therewith.

In certain embodiments, the VH domain comprises at least two of the FWR1, FWR2, FWR3 and FWR4.

In certain embodiments, it comprises at least 3 of them. In certain embodiments, it comprises all 4 of them.

In certain embodiments, the VL domain additionally comprises at least one of:
(a) FWR1 comprising the amino acid sequence DIQMTQSPASLSASLGETVTIECRAS (SEQ ID NO: 48) or a sequence having at least 50% sequence identity therewith,
(b) FWR2 comprising the amino acid sequence LAWYQQKPGDSPQLLIY (SEQ ID NO: 50) or a sequence having at least 50% sequence identity therewith,
(c) FWR3 comprising the amino acid sequence RLAEGVPSRFSGSGSGTQYSLKINSLHSEDVASYFC (SEQ ID NO: 52) or a sequence having at least 50% sequence identity therewith, and
(d) FWR4 comprising the amino acid sequence FGVGTKLDLK (SEQ ID NO: 54) or a sequence having at least 50% sequence identity therewith.

In certain embodiments, the VL domain comprises at least two of the FWR1, FWR2, FWR3 and FWR4. In certain embodiments, it comprises at least 3 of them. In certain embodiments, it comprises all 4 of them.

In certain embodiments, each included FWR domain has at least 60% sequence identity with the corresponding SEQ ID NO. In certain embodiments, each included FWR domain has at least 70% sequence identity with the corresponding SEQ ID NO. In certain embodiments, each included FWR domain has at least 75% sequence identity with the corresponding SEQ ID NO. In certain embodiments, each included FWR domain has at least 80% sequence identity with the corresponding SEQ ID NO. In certain embodiments, each included FWR domain has at least 85% sequence identity with the corresponding SEQ ID NO. In certain embodiments, each included FWR domain has at least 90% sequence identity with the corresponding SEQ ID NO. In certain embodiments, each included FWR domain has at least 95% sequence identity with the corresponding SEQ ID NO. In certain embodiments, each included FWR has at least 96% sequence identity with the corresponding SEQ ID NO. In certain embodiments, each included FWR has at least 97% sequence identity with the corresponding SEQ ID NO. In certain embodiments, each included FWR has at least 98% sequence identity with the corresponding SEQ ID NO. In certain embodiments, each included FWR has at least 99% sequence identity with the corresponding SEQ ID NO.

In certain embodiments, the FWRs are humanized.

In certain embodiments, the VH domain has a sequence identity with the sequence of SEQ ID NO: 38 at least that of one of 75%, 80%, 85%, 90%, 95%, 96%, 97%, 98% and 99%. In certain embodiments, the VH domain comprises the sequence of SEQ ID NO: 38, which can be encoded by SEQ ID NO: 37.

TABLE 29
SEQ ID NO: 37

atggctgtcctggtgctgttgctctgcctggtgacatttccaacctgtg
tcctgtcccaggtgcagctgaaggagtcaggacctggtctggtgcagcc
ctcacagaccctgtccctcacctgcactgtctctggattctcactagac
aactacaatgtgcactgggttcgacagcctacaggaggaggtctggagt
ggttgggaggaatttggactgatggaatcacagactacacttcacctct
cagatcccgactgagcatcagcagggacacctccaagagccaggttttc
ttaaagatgaagagtctgcaaactgaggacatagccacttactactgtg
ccaggtcttattactattccctatactactttgattactggggccaagg
agtcatggtcacagtctcctcag

TABLE 30
SEQ ID NO: 38

QVQLKESGPGLVQPSQTLSLTCTVSGESLDNYNVHWVRQPTGGGLEWLG
GIWTDGITDYTSPLRSRLSISRDTSKSQVFLKMKSLQTEDIATYYCARS
YYYSLYYFDYWGQGVMVTVSS

In certain embodiments, the VL domain has a sequence identity with the sequence of SEQ ID NO: 47 at least that of one of 75%, 80%, 85%, 90%, 95%, 96%, 97%, 98% and 99%. In certain embodiments, the VL domain comprises the sequence of SEQ ID NO: 47, which can be encoded by SEQ ID NO: 46.

TABLE 31
SEQ ID NO: 46

atgggtgtccccactcagctcctggggttgttgctactgtggattacag
atgccatatgtgacatccagatgacacagtctccagcttccctgtctgc
atctctgggagaaactgtcaccatcgaatgtcgagcaagtgaggacatt
tacagtaatttagcgtggtatcagcagaaaccaggggactctcctcagc
tcctgatctatgatgcacataggttggcagagggggtcccatcacggtt
cagtggcagtggatctggcacacagtattctctaaagataaacagcctg
cactctgaagatgtcgcaagttatttctgtcaacaatatgacaattata
acacgtttggagttgggaccaagctggacctgaaac

TABLE 32
SEQ ID NO: 47

DIQMTQSPASLSASLGETVTIECRASEDIYSNLAWYQQKPGDSPQLLIY
DAHRLAEGVPSRFSGSGSGTQYSLKINSLHSEDVASYFCQQYDNYNTFG
VGTKLDLK

As detailed below, these sequences are from the mouse 2B11 monoclonal antibody raised against human IL-11 and which displays excellent binding characteristics.

In certain embodiments, antibody comprises the VH domain of SEQ ID NO: 38 and the VL domain of SEQ ID NO: 47. An exemplary antibody including those domains is CSJ-A-s-2B11 (details below).

TABLE 33
CDR and FWR sequences of 2B11 HC (Heavy Chain) and LC (Light Chain):

| | SEQ ID | | SEQ ID |
|---|---|---|---|
| HC | NO | LC | NO |
| FWR1: QVQLKESGPG | 39 | FWR1: DIQMTQSPAS | 48 |
| LVQPSQTLSL TCTVS | | LSASLGETVT IECRAS | |
| CDR1: GFSLDNYN | 40 | CDR1: EDIYSN | 49 |
| FWR2: VHWVRQPTGG GLEWLGG | 41 | FWR2: LAWYQQKPGD SPQLLIY | 50 |
| CDR2: IWTDGIT | 42 | CDR2: DAH | — |
| FWR3: DYTSPLRSRL | 43 | FWR3: RLAEGVPSRF | 52 |
| SISRDTSKSQ VFLKMKSLQT | | SGSGSGTQYS LKINSLHSED | |
| EDIATYYC | | VASY FC | |
| CDR3: ARSYYYSLYY FDY | 44 | CDR3: QQYDNYNT | 53 |
| FWR4: WGQGVMVTVS S | 45 | FWR4: FGVGTKLDLK | 54 |

The 2B11 light chain CDR2 sequence is 3 amino acids in length and as such cannot be represented in accordance with WIPO standard ST.26 (which sets a minimum amino acid sequence length of 4). For the avoidance of doubt, any reference to SEQ ID NO: 51 is reference to the amino acid sequence DAH.

The full VH and VL sequences are given in SEQ ID NOs: 37, 38, 46 and 47. The nucleotide sequences detail the location and identity of domains (FWRs, CDRs) within them. 10D10: According to a fourth aspect of the present invention there is provided an antibody that binds to human IL-11, the antibody comprising a heavy chain (HC) variable region sequence and a light chain (LC) variable region sequence,
(i) the heavy chain variable region sequence comprising the CDR3 and at least one additional CDR selected from the group consisting of:
   (a) HC-CDR1 comprising the amino acid sequence GFSLTNYH (SEQ ID NO: 58) or a variant having one, two or three amino acid substitutions therein,
   (b) HC-CDR2 comprising the amino acid sequence MWIDGDT (SEQ ID NO: 60) or a variant having one or two amino acid substitutions therein, and
   (c) HC-CDR3 comprising the amino acid sequence AREGLNWFAY (SEQ ID NO: 62) or a variant having one or two amino acid substitutions therein; and
(ii) the light chain variable region sequence comprising the CDR3 and at least one additional CDR selected from the group consisting of:
   (a) LC-CDR1 comprising the amino acid sequence QGIDDY (SEQ ID NO: 67) or a variant having one or two amino acid substitutions therein,
   (b) LC-CDR2 comprising the amino acid sequence DVT or a variant having one amino acid substitution therein, and
   (c) LC-CDR3 comprising the amino acid sequence LQSYSTPYT (SEQ ID NO: 71) or a variant having one or two amino acid substitutions therein.

In certain embodiments, the VH domain comprises all three of the CDR1, CDR2 and CDR3. In certain embodiments, the VL domain comprises all three of the CDR1, CDR2 and CDR3. In certain embodiments, the VH domain comprises all three of the CDR1, CDR2 and CDR3, and the VL domain comprises all three of the CDR1, CDR2 and CDR3.

In certain embodiments, the VH domain additionally comprises at least one of:
(a) FWR1 comprising the amino acid sequence QVQLKESGPGLVQPSQTLSLTCTVS (SEQ ID NO: 57) or a sequence having at least 50% sequence identity therewith,
(b) FWR2 comprising the amino acid sequence VHWVRQPPGKGLEWMGV (SEQ ID NO: 59) or a sequence having at least 50% sequence identity therewith,
(c) FWR3 comprising the amino acid sequence SYNSALRSRLSISRDTSKSQVFLKMSSLQTEDTATYYC (SEQ ID NO: 61) or a sequence having at least 50% sequence identity therewith, and
(d) FWR4 comprising the amino acid sequence WGRGTLVTVSS (SEQ ID NO: 63) or a sequence having at least 50% sequence identity therewith.

In certain embodiments, the VH domain comprises at least two of the FWR1, FWR2, FWR3 and FWR4.

In certain embodiments, it comprises at least 3 of them. In certain embodiments, it comprises all 4 of them.

In certain embodiments, the VL domain additionally comprises at least one of:
(a) FWR1 comprising the amino acid sequence DIQMTQSPASLSVSLEEVVTITCKAS (SEQ ID NO: 66) or a sequence having at least 50% sequence identity therewith,
(b) FWR2 comprising the amino acid sequence LSWYQQKPGKSPQLLIS (SEQ ID NO: 68) or a sequence having at least 50% sequence identity therewith,
(c) FWR3 comprising the amino acid sequence SLADGVPSRFSGSRSGTQYSLKISRPQVDDSGIYYC (SEQ ID NO: 70) or a sequence having at least 50% sequence identity therewith, and
(d) FWR4 comprising the amino acid sequence FGAGTKLELK (SEQ ID NO: 72) or a sequence having at least 50% sequence identity therewith.

In certain embodiments, the VL domain comprises at least two of the FWR1, FWR2, FWR3 and FWR4. In certain embodiments, it comprises at least 3 of them. In certain embodiments, it comprises all 4 of them.

In certain embodiments, each included FWR domain has at least 60% sequence identity with the corresponding SEQ ID NO. In certain embodiments, each included FWR domain has at least 70% sequence identity with the corresponding SEQ ID NO. In certain embodiments, each included FWR domain has at least 75% sequence identity with the corresponding SEQ ID NO. In certain embodiments, each included FWR domain has at least 80% sequence identity with the corresponding SEQ ID NO. In certain embodiments, each included FWR domain has at least 85% sequence identity with the corresponding SEQ ID NO. In certain embodiments, each included FWR domain has at least 90% sequence identity with the corresponding SEQ ID NO. In certain embodiments, each included FWR domain has at least 95% sequence identity with the corresponding SEQ ID NO. In certain embodiments, each included FWR has at least 96% sequence identity with the corresponding SEQ ID NO. In certain embodiments, each included FWR has at least 97% sequence identity with the corresponding SEQ ID NO. In certain embodiments, each included FWR has at least 98% sequence identity with the corresponding SEQ ID NO. In certain embodiments, each included FWR has at least 99% sequence identity with the corresponding SEQ ID NO.

In certain embodiments, the FWRs are humanized.

In certain embodiments, the VH domain has a sequence identity with the sequence of SEQ ID NO: 56 at least that of one of 75%, 80%, 85%, 90%, 95%, 96%, 97%, 98% and 99%. In certain embodiments, the VH domain comprises the sequence of SEQ ID NO: 56, which can be encoded by SEQ ID NO: 55.

TABLE 34
SEQ ID NO: 55

atggctgtcctggtgctgttgctctgcctgatgacatttccaagctgtg
tcctgtcccaggtgcagctgaaggagtcaggacctggcctggtgcagcc
ctcacagaccctgtctctcacctgcactgtctctgggttctcattaacc
aattatcatgtgcactgggttcgacagcctccaggaaaaggtctggagt
ggatgggagtaatgtggattgatggcgacacatcatataattcagctct
cagatcccgactgagcatcagcagggacacctccaagagccaagttttc
ttaaaaatgagcagtctgcagactgaagacacagccacttactactgtg
ccagagagggtctcaattggtttgcttactggggccgaggcactctggt
cactgtctcttcag

TABLE 35
SEQ ID NO: 56

QVQLKESGPGLVQPSQTLSLTCTVSGFSLTNYHVHWVRQPPGKGLEWMG
VMWIDGDTSYNSALRSRLSISRDTSKSQVFLKMSSLQTEDTATYYCARE
GLNWFAYWGRGTLVTVSS

In certain embodiments, the VL domain has a sequence identity with the sequence of SEQ ID NO: 65 at least that of one of 75%, 80%, 85%, 90%, 95%, 96%, 97%, 98% and 99%. In certain embodiments, the VL domain comprises the sequence of SEQ ID NO: 65, which can be encoded by SEQ ID NO: 64.

TABLE 36
SEQ ID NO: 64

atgaacgtgcccactcaactccttgggttgctgctactctggcttacag
gtggtaaatgtgacatccagatgacacagtctcctgcctccctgtctgt
ttctctggaagaagttgtcaccatcacctgcaaggcaagccagggcatt
gatgattacttatcatggtatcagcagaaaccagggaaatctcctcagc
tcctgatctctgatgtaaccagcttggcagatggggtcccatcacggtt
cagcggcagtagatctggcacacagtattctcttaagatcagcagacca
caggttgatgattctggaatctattactgtctacagagttacagtactc
cgtacacgtttggagctgggaccaagctggaactgaaac

TABLE 37
SEQ ID NO: 65

DIQMTQSPASLSVSLEEVVTITCKASQGIDDYLSWYQQKPGKSPQLLIS
DVTSLADGVPSRFSGSRSGTQYSLKISRPQVDDSGIYYCLQSYSTPYTF
GAGTKLELK

As detailed below, these sequences are from the mouse 10D10 monoclonal antibody raised against human IL-11 and which displays excellent binding characteristics.

In certain embodiments, the antibody comprises the VH domain of SEQ ID NO: 56 and the VL domain of SEQ ID NO: 65. An exemplary antibody including those domains is CSJ-A-s-10D10 (details below).

TABLE 38
CDR and FWR sequences of 10D10 HC (Heavy Chain) and LC (Light Chain):

| | SEQ ID | | SEQ ID |
|---|---|---|---|
| HC | NO | LC | NO |
| FWR1: QVQLKESGPG | 57 | FWR1: DIQMTQSPAS | 66 |
| LVQPSQTLSL TCTVS | | LSVSLEEVVT ITCKAS | |
| CDR1: GFSLTNYH | 58 | CDR1: QGIDDY | 67 |
| FWR2: VHWVRQPPGK GLEWMGV | 59 | FWR2: LSWYQQKPGK SPQLLIS | 68 |
| CDR2: MWIDGDT | 60 | CDR2: DVT | — |
| FWR3: SYNSALRSRL | 61 | FWR3: SLADGVPSRF | 70 |
| SISRDTSKSQ VFLKMSSLQT | | SGSRSGTQYS LKISRPQVDD | |
| EDTATYYC | | SGIYYC | |
| CDR3: AREGLNWFAY | 62 | CDR3: LQSYSTPYT | 71 |
| FWR4: WGRGTLVTVS S | 63 | FWR4: FGAGTKLELK | 72 |

The 10D1D light chain CDR2 sequence is 3 amino acids in length and as such cannot be represented in accordance with WIPO standard ST.26 (which sets a minimum amino acid sequence length of 4). For the avoidance of doubt, any reference to SEQ ID NO: 69 is reference to the amino acid sequence DVT.

The full VH and VL sequences are given in SEQ ID NOs: 55, 56, 64 and 65. The WIPO ST.26 formatted nucleotide sequences detail the location and identity of domains (FWRs, CDRs) within them.

### 3G8:

According to a fifth aspect of the present invention there is provided an antibody that binds to human IL-11, the antibody comprising a heavy chain (HC) variable region sequence and a light chain (LC) variable region sequence,
(i) the heavy chain variable region sequence comprising the CDR3 and at least one additional CDR selected from the group consisting of:
   (a) HC-CDR1 comprising the amino acid sequence GFSMSTSGMV (SEQ ID NO: 76) or a variant having one, two or three amino acid substitutions therein,
   (b) HC-CDR2 comprising the amino acid sequence IDWDGEK (SEQ ID NO: 78) or a variant having one or two amino acid substitutions therein, and
   (c) HC-CDR3 comprising the amino acid sequence ARSGNYFDY (SEQ ID NO: 80) or a variant having one or two amino acid substitutions therein; and
(ii) the light chain variable region sequence comprising the CDR3 and at least one additional CDR selected from the group consisting of:
   (a) LC-CDR1 comprising the amino acid sequence EDIYNG (SEQ ID NO: 85) or a variant having one or two amino acid substitutions therein,
   (b) LC-CDR2 comprising the amino acid sequence DAN or a variant having one amino acid substitution therein, and
   (c) LC-CDR3 comprising the amino acid sequence QQYYDYPNT (SEQ ID NO: 89) or a variant having one or two amino acid substitutions therein.

In certain embodiments, the VH domain comprises all three of the CDR1, CDR2 and CDR3. In certain embodiments, the VL domain comprises all three of the CDR1, CDR2 and CDR3. In certain embodiments, the VH domain comprises all three of the CDR1, CDR2 and CDR3, and the VL domain comprises all three of the CDR1, CDR2 and CDR3.

In certain embodiments, the VH domain additionally comprises at least one of:
(a) FWR1 comprising the amino acid sequence QVTLKESGPGMLQPSKTVSLTCSFS (SEQ ID NO: 75) or a sequence having at least 50% sequence identity therewith,
(b) FWR2 comprising the amino acid sequence VSWIRQPSGKSLEWLAA (SEQ ID NO: 77) or a sequence having at least 50% sequence identity therewith,
(c) FWR3 comprising the amino acid sequence YYNPSLKSRLTVSKDTSNTQVFLKISSVDIADSATYYC (SEQ ID NO: 79) or a sequence having at least 50% sequence identity therewith, and
(d) FWR4 comprising the amino acid sequence WGQGVMVTVSS (SEQ ID NO: 81) or a sequence having at least 50% sequence identity therewith.

In certain embodiments, the VH domain comprises at least two of the FWR1, FWR2, FWR3 and FWR4. In certain embodiments, it comprises at least 3 of them. In certain embodiments, it comprises all 4 of them.

In certain embodiments, the VL domain additionally comprises at least one of:
(a) FWR1 comprising the amino acid sequence DIQMTQSPASLSASLGETVTIECRAS (SEQ ID NO: 84) or a sequence having at least 50% sequence identity therewith,
(b) FWR2 comprising the amino acid sequence LVWYQQKPGKSPQLLIY (SEQ ID NO: 86) or a sequence having at least 50% sequence identity therewith,
(c) FWR3 comprising the amino acid sequence RLHTGVPSRFSGGGSGTQYSLKINSLQSEDVASYFC (SEQ ID NO: 88) or a sequence having at least 50% sequence identity therewith, and
(d) FWR4 comprising the amino acid sequence FGAGTKLELK (SEQ ID NO: 90) or a sequence having at least 50% sequence identity therewith.

In certain embodiments, the VL domain comprises at least two of the FWR1, FWR2, FWR3 and FWR4. In certain embodiments, it comprises at least 3 of them. In certain embodiments, it comprises all 4 of them.

In certain embodiments, each included FWR domain has at least 60% sequence identity with the corresponding SEQ ID NO. In certain embodiments, each included FWR domain has at least 70% sequence identity with the corresponding SEQ ID NO. In certain embodiments, each included FWR domain has at least 75% sequence identity with the corresponding SEQ ID NO. In certain embodiments, each included FWR domain has at least 80% sequence identity with the corresponding SEQ ID NO. In certain embodiments, each included FWR domain has at least 85% sequence identity with the corresponding SEQ ID NO. In certain embodiments, each included FWR domain has at least 90% sequence identity with the corresponding SEQ ID NO. In certain embodiments, each included FWR domain has at least 95% sequence identity with the corresponding SEQ ID NO. In certain embodiments, each included FWR has at least 96% sequence identity with the corresponding SEQ ID NO. In certain embodiments, each included FWR has at least 97% sequence identity with the corresponding SEQ ID NO. In certain embodiments, each included FWR has at least 98% sequence identity with the corresponding SEQ ID NO. In certain embodiments, each included FWR has at least 99% sequence identity with the corresponding SEQ ID NO.

In certain embodiments, the FWRs are humanized.

In certain embodiments, the VH domain has a sequence identity with the sequence of SEQ ID NO: 74 at least that of one of 75%, 80%, 85%, 90%, 95%, 96%, 97%, 98% and 99%. In certain embodiments, the VH domain comprises the sequence of SEQ ID NO: 74, which can be encoded by SEQ ID NO: 73.

TABLE 39
SEQ ID NO: 73

atggacaggcttacttcctcattcctgctgctgcttgtccctgcatatg
tcttgtcccaggttactctgaaagagtctggtcctgggatgttacagcc
ctccaagaccgtaagcctgacttgctctttttctgggttttcaatgagc
acttctggtatggttgtgagctggattcgtcagccttcagggaagagtc
tcgagtggctggcagccattgattgggatggtgaaaagtactacaaccc
atctctgaaaagcaggctcacagtctccaaggacacctccaacacccaa
gtgttcctcaagatcagcagtgtggacattgcagatagtgccacatact
actgtgctcggagtggtaactactttgattactggggccaaggagtcat
ggtcacagtctcctcag

TABLE 40
SEQ ID NO: 74

QVTLKESGPGMLQPSKTVSLTCSFSGFSMSTSGMVVSWIRQPSGKSLEW
LAAIDWDGEKYYNPSLKSRLTVSKDTSNTQVFLKISSVDIADSATYYCA
RSGNYFDYWGQGVMVTVSS

In certain embodiments, the VL domain has a sequence identity with the sequence of SEQ ID NO: 83 at least that of one of 75%, 80%, 85%, 90%, 95%, 96%, 97%, 98% and 99%. In certain embodiments, the VL domain comprises the sequence of SEQ ID NO: 83, which can be encoded by SEQ ID NO: 82.

TABLE 41
SEQ ID NO: 82

gacatccagatgacacagtctccagcttccctgtctgcatctctgggag
aaactgtcaccatcgaatgtcgagcaagtgaggacatttacaatggttt
agtatggtatcagcagaagccagggaaatctcctcagctcctgatctat
gatgcaaatcgcttgcatactggggtcccatcacggttcagtggcggtg
gatctggtacacagtattctctcaagataaacagcctgcaatctgaaga
tgtcgcaagttatttctgtcaacagtactacgattatccgaacacgttt
ggagctgggaccaagctggagctgaaac

TABLE 42
SEQ ID NO: 83

DIQMTQSPASLSASLGETVTIECRASEDIYNGLVWYQQKPGKSPQLLIY
DANRLHTGVPSRFSGGGSGTQYSLKINSLQSEDVASYFCQQYYDYPNTF
GAGTKLELK

As detailed below, these sequences are from the mouse 3G8 monoclonal antibody raised against human IL-11 and which displays excellent binding characteristics.

In certain embodiments, the antibody comprises the VH domain of SEQ ID NO: 74 and the VL domain of SEQ ID NO: 83. An exemplary antibody including those domains is CSJ-B-s-3G8 (details below).

TABLE 43
CDR and FWR sequences of 3G8 HC (Heavy Chain) and LC (Light Chain):

| | SEQ ID | | SEQ ID |
|---|---|---|---|
| HC | NO | LC | NO |
| FWR1: QVTLKESGPG | 75 | FWR1: DIQMTQSPAS | 84 |
| MLQPSKTVSL TCSES | | LSASLGETVT IECRAS | |
| CDR1: GFSMSTSGMV | 76 | CDR1: EDIYNG | 85 |
| FWR2: VSWIRQPSGK SLEWLAA | 77 | FWR2: LVWYQQKPGK SPQLLIY | 86 |
| CDR2: IDWDGEK | 78 | CDR2: DAN | — |
| FWR3: YYNPSLKSRL | 79 | FWR3: RLHTGVPSRF | 88 |
| TVSKDTSNTQ VFLKISSVDI | | SGGGSGTQYS LKINSLQSED | |
| ADSATYYC | | VASYFC | |
| CDR3: ARSGNYFDY | 80 | CDR3: QQYYDYPNT | 89 |
| FWR4: WGQGVMVTVS S | 81 | FWR4: FGAGTKLELK | 90 |

The 3G8 light chain CDR2 sequence is 3 amino acids in length and as such cannot be represented in accordance with WIPO standard ST.26 (which sets a minimum amino acid sequence length of 4). For the avoidance of doubt, any reference to SEQ ID NO: 87 is reference to the amino acid sequence DAN.

The full VH and VL sequences are given in SEQ ID NOs: 73, 74, 82 and 83. The WIPO ST.26 formatted nucleotide sequences detail the location and identity of domains (FWRs, CDRs) within them.

### Epitope Mapping and Additional Antibodies

As detailed in the Experiments section below, epitope mapping has been undertaken based on various of the monoclonal antibodies and has resulted in the identification of a number of discontinuous epitopes. With that information (and as detailed below), antibodies specific against the identified epitopes can be readily produced using standard techniques and procedures.

Thus, also provided according to the present invention is an antibody or an immunologically active fragment thereof that binds to human IL-11, wherein the antibody binds to a discontinuous epitope on human IL-11, wherein the discontinuous epitope comprises amino acid residues:
(a) R30, S41, T95, R98, S145, and R146; (“CSI-s-9B9 discontinuous epitope”)
(b) R30, 541, R138, R146, R190, K196, T197, and R198; (“CSJ-A-s-2B11 discontinuous epitope”)
(c) R30, T46, R135, 5145, R146, R190, and K196; (“CSJ-A-s-10D10 discontinuous epitope”)
(d) S48, T53, R54, R96, R98, S103, R106, H107, T128, R132, R138, R139, H182, and R190; (“CSJ-B-s-3F6 discontinuous epitope”) or
(e) T46, T54, T95, R112, H182, and R190; (“CSJ-B-s-3G8 discontinuous epitope”) when numbered in accordance with SEQ ID NO: 125.

The sequence of SEQ ID NO: 125 is the consensus hIL-11 sequence having NCBI (National Centre for Biotechnology Information, National Library of Medicine, USA) accession/version number NP_000632.1(www.ncbi.nlm.nih.gov/protein/NP 000632.1/)

Additional antibodies specific against the discontinuous epitopes of the present invention can be readily produced using standard techniques, for example as detailed in the “Production of further antibodies specific against identified epitopes” section below.

The antibody of the present invention can be a monoclonal antibody.

In certain embodiments, the antibody is chemically or biologically modified. In certain embodiments, the antibody is glycosylated, PEGylated or HESylated.

As detailed in the experiments below (see in particular Experiment 2 and Experiment 6 and the results in Table 44 and Table 49), antibodies according to the present invention display excellent binding characteristics. Thus, in certain embodiments, the equilibrium dissociation constant (K_{D}) for binding of human IL11 by the antibody is less than 1 nM (1E-09 M).

Table 44 (below) details equilibrium dissociation constant (K_{D}) values for antibodies CSI-s-9B9, CSJ-A-s-2B11, CSJ-A-s-10D10, CSJ-B-3F6, and CSJ-B-s-3G8. All are less than 1 nM (1E-09 M).

Table 49 (below) details equilibrium dissociation constant (K_{D}) values for antibodies GVH5 (D54E)/CVL3, GVH5 (D54E)/GVLS and GVH5 (D54E)/GVLS+YTE. All are less than 100 pM (1E-10 M).

Table 49 (below) also details equilibrium dissociation constant (K_{D}) values for antibodies VH0/VL0, CVH3 (D54E)/CVL3, CVH3 (D54E)/CVL3+YTE, CVH4 (D54E)/CVL3, CVH4 (D54E)/CVL3+YTE, and GVH5 (D54E)/CVL3+YTE. All are less than 10 pM (1E-11M).

Thus, in certain embodiments, the equilibrium dissociation constant (K_{D}) for binding of human IL11 by the antibody is less than 100 pM (1E-10 M).

In certain embodiments, the equilibrium dissociation constant (K_{D}) for binding of human IL11 by the antibody is less than 50 pM (5E-11 M).

In certain embodiments, the equilibrium dissociation constant (K_{D}) for binding of human IL11 by the antibody is less than 25 pM (2.5E-11 M).

In certain embodiments, the equilibrium dissociation constant (K_{D}) for binding of human I11 by the antibody is less than 20 pM (2E-11 M).

In certain embodiments, the equilibrium dissociation constant (K_{D}) for binding of human IL11 by the antibody is less than 10 pM (1E-11 M).

In certain embodiments, the equilibrium dissociation constant (K_{D}) for binding of human IL11 by the antibody is less than 5 pM (5E-12).

In certain embodiments, the equilibrium dissociation constant (K_{D}) for binding of human IL11 by the antibody is less than 2 pM (2E-12).

In certain embodiments, the equilibrium dissociation constant (K_{D}) for binding of human IL11 by the antibody is less than 1.5 pM (1.5E-12).

The equilibrium dissociation constant can be determined using Surface Plasmon Resonance (SPR).

In certain embodiments, analysis is done at 25DegC.

In certain embodiments, the human IL-11 analyte is Human IL-11 (Genscript, NJ, USA; Cat. No. Z03108).

In certain embodiments, the duration of the association phase (the association time) is between 180 and 240 seconds. In certain embodiments, it is between 190 and 230 seconds. In certain embodiments, it is between 200 and 220 seconds. In certain embodiments, it is 210 seconds.

In certain embodiments, the duration of the dissociation phase (the dissociation time) is between 800 and 1000 seconds. In certain embodiments, it is between 825 and 975 seconds. In certain embodiments, it is between 850 and 950 seconds. In certain embodiments, it is between 875 and 925 seconds. In certain embodiments, it is 900 seconds.

In certain embodiments, the dilution range is an at least two-fold multipoint dilution. In certain embodiments, the dilution range is an at least three-fold multipoint dilution. In certain embodiments, dilutions are done at at least 3 points (i.e. >=3 points). In certain embodiments, dilutions are done at at least 4 points (i.e. >=3 points). In certain embodiments, dilutions are done at at least 5 points (i.e. >=5 points). In certain embodiments, dilutions are done at at least 6 points (i.e. >=6 points). In certain embodiments, dilutions are done at at least 7 points (i.e. >=7 points). In certain embodiments, dilutions are done at at least 8 points (i.e. >=8 points). In certain embodiments, dilutions are done at at least 9 points (i.e. >=9 points).

An exemplary SPR device is the Biacore (RTM) T200 (Cytiva, Marlborough, USA). Such a device can be running Biacore T200 Evaluation Software V2.0.1.

Additional parameters can include one or more of the following:
Running buffer: HBS-P+(Cytiva, Marlborough, USA), 1 mg/mL BSA
Chip: Series S Anti-mouse antibody capture kit
Ligand: Purified antibody loaded to ~160 RU diluted in running buffer to 1.5 Pg/mL
Flow rate: 30 μl/min
Association: 210 s
Dissociation: 900 s
Dilution Range: 6 point three-fold dilution from 90 nM to 0.37 nM (hIL-11)
Regeneration: 10 mM glycine pH 1.7
Analysis: 1:1 binding with double reference subtraction

In certain embodiments, all of the above additional parameters for the Biacore (RTM) T200 can be used.

An alternative exemplary SPR (Surface Plasmon Resonance) device which can be used to determine the binding (i.e. the equilibrium dissociation constant) of antibodies binding to human IL-11 antigen is the Biacore (RTM) 8K. Such a device can be running Biacore 8K Control software V3.0.12.15655 and Biacore Insight Evaluation software V3.0.12.15655 (Cytiva, Uppsala, Sweden).

As detailed in the “Half-life determination” section below, experiments have determined that antibodies according to the present invention display an excellent half-life as determined using hFcRN (humanized FcRn) mice (The Jackson Laboratory, Maine, USA; www.iax.org/fcrn; Roopenian D C et al., Human FcRn transgenic mice for pharmacokinetic evaluation of therapeutic antibodies. Methods Mol Biol. 2010; 602:93-104. doi: 10.1007/978-1-60761-058-8 6. PMID: 20012394). In certain embodiments, the antibody has a half-life of >100 hours when determined by IV injection of a single dose of antibody at 2 nm/kg in hFcRn mice. In certain embodiments, the antibody has a half-life of >200 hours at 2 nm/kg in hFcRn mice. In certain embodiments, the antibody has a half-life of >300 hours at 2 nm/kg in hFcRn mice. In certain embodiments, the antibody has a half-life of >400 hours at 2 nm/kg in hFcRn mice. In certain embodiments, the antibody has a half-life of >450 hours at 2 nm/kg in hFcRn mice. In certain embodiments, the antibody has a half-life of >500 hours at 2 nm/kg in hFcRn mice. In certain embodiments, the antibody has a half-life of >550 hours at 2 nm/kg in hFcRn mice. In certain embodiments, the antibody has a half-life of >575 hours at 2 nm/kg in hFcRn mice.

In certain embodiments, the antibody has a half-life of >100 hours when determined by IV injection of a single dose of antibody at 10 nm/kg in hFcRn mice. In certain embodiments, the antibody has a half-life of >200 hours at 10 nm/kg in hFcRn mice. In certain embodiments, the antibody has a half-life of >225 hours at 10 nm/kg in hFcRn mice. In certain embodiments, the antibody has a half-life of >300 hours at 10 nm/kg in hFcRn mice. In certain embodiments, the antibody has a half-life of >400 hours at 10 nm/kg in hFcRn mice. In certain embodiments, the antibody has a half-life of >450 hours at 10 nm/kg in hFcRn mice. In certain embodiments, the antibody has a half-life of >500 hours at 10 nm/kg in hFcRn mice. In certain embodiments, the antibody has a half-life of >525 hours at 10 nm/kg in hFcRn mice.

Also provided according to the present invention is an isolated nucleic acid molecule encoding the antibody or an antigen binding fragment thereof according to the present invention.

Also provided according to the present invention is a vector comprising a nucleic acid molecule according to the present invention.

Also provided according to the present invention is a host cell comprising a nucleic acid molecule or a vector according to the present invention.

Also provided according to the present invention is a composition comprising an antibody or antigen binding fragment thereof, nucleic acid molecule, vector or host cell according to the present invention, in combination with a suitable carrier, diluent or excipient.

The composition can be a pharmaceutical composition, and the carrier, diluent or excipient can be a pharmaceutically acceptable carrier, diluent or excipient.

Carriers, diluents and excipients are well known in the art and include those taught in Remington, The Science and Practice of Pharmacy, 2020, ISBN: 9780128200070.

The pharmaceutical composition can be in a form suitable for parenteral, oral, rectal, systemic, urogenital, topical, intravitreal, intraocular, otic, intranasal, dermal, sublingual, or buccal administration.

Also provided according to the present invention is an antibody, nucleic acid molecule, vector, host cell or composition according to the present invention, for use in a method of treatment or diagnosis of the human or animal body. In particular, this can be a method of treatment or diagnosis of the human body. As detailed above, anti-IL-11 antibodies are useful in therapeutic and diagnostic methods. Similarly, nucleic acid molecules, vectors, and host cells can also be used in methods of treatment or diagnosis.

Also provided according to the present invention is a diagnostic test method for human IL-11, the method comprising the steps of:
i) reacting an antibody according to the present invention that binds to human IL-11 with serum from a patient;
ii) detecting any antibody-antigen binding reaction; and
iii) correlating detection of the antibody-antigen binding reaction with the presence of human IL-11.

In certain embodiments, the diagnostic test method comprises quantifying the antibody-antigen binding.

### DRAWINGS

FIG. 1A shows absorbance results using MAB218 reference anti-human-IL-11 antibody with a range of concentrations of human IL-11 coated on ELISA plates;

FIG. 1B shows the results of FIG. 1A but just for 0.25 g/ml and 0 μg/ml;

FIGS. 2A-2D show results of antigen binding ELISA assays for reference antibody MAB218 (FIGS. 2A and 2C), and antibodies CSI-s-6H9, CSI-s-7H3-E4, and CSI-s-9B9;

FIG. 3 shows antigen binding ELISA results;

FIG. 4 shows hlL-11 titration;

FIG. 5 shows titration of MAB218;

FIGS. 6A-6D show inhibition of rhlL-11induced STAT3 activity by humanised clones;

FIG. 6E shows summary results data from FIGS. 6A-6D;

FIG. 7 shows sequences for SEQ ID NOs: 91-100 and 124;

FIG. 8 shows sequences for SEQ ID NOs: 116-122;

FIGS. 9A-9B show results of in vivo testing of the efficacy of various IL-11 mAb clones in preventing acetaminophen-induced liver injury (AILI);

FIGS. 10A-10B show results of in vivo testing of the efficacy of various IL-11 mAb clones in preventing acetaminophen-induced livery injury (AILI);

FIG. 11 shows results of high-mass MALDI analysis on control and cross-link samples of IL-11_Human;

FIG. 12 shows results of high-mass MALDI analysis on control and cross-link samples of CSI-s-9B9;

FIG. 13 shows results of high-mass MALDI analysis on control and cross-link samples of CSJ-A-s-2B11;

FIG. 14 shows results of high-mass MALDI analysis on control and cross-link samples of CSJ-A-s-10D10;

FIG. 15 shows results of high-mass MALDI analysis on control and cross-link samples of CSJ-B-3F6;

FIG. 16 shows results of high-mass MALDI analysis on control and cross-link samples of CSJ-B-s-3G8;

FIG. 17 shows results of high-mass MALDI analysis on control and cross-link samples of IL-11_Human and CSI-s-9B9;

FIG. 18 shows results of high-mass MALDI analysis on control and cross-link samples of IL-11_Human and CSJ-A-s-2B11;

FIG. 19 shows results of high-mass MALDI analysis on control and cross-link samples of IL-11_Human and CSJ-A-s-10D10;

FIG. 20 shows results of high-mass MALDI analysis on control and cross-link samples of IL-11_Human and CSJ-B-3F6;

FIG. 21 shows results of high-mass MALDI analysis on control and cross-link samples of IL-11_Human and CSJ-B-s-3G8;

FIG. 22 shows an overlap mapping of the peptides obtained by proteolysis using Trypsin, Chymotrypsin, ASP-N, Elastase and Thermolysin peptides as compared to the source (undigested) molecule IL-11_Human;

FIG. 23 shows (top) a chromatogram of IL-11_Human trypsin digest, and (bottom) sum of total ions of a chromatogram of nLC IL-11_Human trypsin digest;

FIG. 24 shows interaction of CSI-s-9B9 and IL-11_Human;

FIG. 25 shows IL-11_Human/CSI-s-9B9. IL-11_Human PDB structure 4MHL was coloured in blue on the epitope site (dark shading in black & white images). Coloured IL-11_Human amino acids correspond to residues 30-41, 95-98, and 145-146 of SEQ ID NO: 125. A, B, C, D, E: ribbon/surface representation of front view (A); back view (B), side view 1 (C), side view 2 (D), and top view (E). F, G, H, I, J: ribbon representation of front view (F); back view (G), side view 1 (H), side view 2 (1), and top view (J);

FIG. 26 shows interaction of CSJ-A-s-2B11 and IL-11_Human;

FIG. 27 shows IL-11_Human/CSJ-A-s-2B11. IL-11_Human PDB structure 4MHL was coloured in blue on the epitope site (dark shading on the black & white images). Coloured IL-11_Human amino acids correspond to residues 30-41, 138-146, and 190-198. A, B, C, D, E: ribbon/surface representation of front view (A); back view (B), side view 1 (C), side view 2 (D), and top view (E). F, G, H, I, J: ribbon representation of front view (F); back view (G), side view 1 (H), side view 2 (1), and top view (J);

FIG. 28 shows interaction of CSJ-A-s-10D10 and IL-11_Human;

FIG. 29 shows IL-11_Human/CSJ-A-s-10D10. IL-11_Human PDB structure 4MHL was coloured in blue on the epitope site (dark shading in black & white images). Coloured IL-11_Human amino acids correspond to residues 30-46, 135-146, and 190-196 of SEQ ID NO: 125. A, B, C, D, E: ribbon/surface representation of front view (A); back view (B), side view 1 (C), side view 2 (D) and top view (E). F, G, H, I, J: ribbon representation of front view (F); back view (G), side view 1 (H), side view 2 (1) and top view (J);

FIG. 30 shows interaction CSJ-B-s-3F6 and IL-11_Human;

FIG. 31 shows IL-11_Human/CSJ-B-s-3F6. IL-11_Human PDB structure 4MHL was coloured in blue on the epitope site (dark shading on the black & white images). Coloured IL-11_Human amino acids correspond to residues 48-54, 96-107, 128-139, and 182-190. A, B, C, D, E: ribbon/surface representation of front view (A); back view (B), side view 1 (C), side view 2 (D) and top view (E). F, G, H, I, J: ribbon representation of front view (F); back view (G), side view 1 (H), side view 2 (1) and top view (J);

FIG. 32 shows interaction of CSJ-B-s-3G8 and IL-11_Human;

FIG. 33 shows IL-11_Human/CSJ-B-s-3G8. IL-11_Human PDB structure 4MHL was coloured in blue on the epitope site (dark shading in black & white images). Coloured IL-11_Human amino acids correspond to residues 46-54, 95-112, and 182-190 of SEQ ID NO: 125. A, B, C, D, E: ribbon/surface representation of front view (A); back view (B), side view 1 (C), side view 2 (D) and top view (E). F, G, H, I, J: ribbon representation of front view (F); back view (G), side view 1 (H), side view 2 (1) and top view (J); and

FIG. 34 shows the sequence of SEQ ID NO: 125.

### SPECIFIC EMBODIMENTS

Unless defined otherwise, all technical and scientific terms used herein have the same meaning as commonly understood by one of ordinary skill in the art.

The use of the word “a” or “an” when used herein m conjunction with the term “comprising” may mean “one”, but it is also consistent in certain embodiments with the meaning of “one or more”, “at least one”, or “one or more than one”, i.e. “comprising” as used herein to specify the inclusion of components also includes embodiments in which no further components are present.

As used herein, the terms “comprising”, “having”, “including”, and “containing” and grammatical variations thereof, are inclusive or open-ended and do not exclude additional, unrecited elements and/or method steps. The term “consisting essentially of” when used herein in connection with a composition, use or method, denotes that additional elements and/or method steps may be present, but that these additions do not materially affect the manner in which the recited composition, method or use functions. The term “consisting of” when used herein in connection with a composition, use or method, excludes the presence of additional elements and/or method steps. A composition, use or method described herein as comprising certain elements and/or steps may also, in certain embodiments consist essentially of those elements and/or steps, and in other embodiments consist of those elements and/or steps, whether or not these embodiments are specifically referred to.

Unless the context dictates otherwise, particular features, structures and/or characteristics described in connection with an embodiment disclosed herein may be combined with features, structures and/or characteristics described in connection with another embodiment disclosed herein in any suitable manner to provide one or more further embodiments.

It is also to be understood that the positive recitation of a feature in one embodiment, serves as a basis for excluding the feature in an alternative embodiment. For example, where a list of options is presented for a given embodiment or claim, it is to be understood that one or more option may be deleted from the list and the shortened list may form an alternative embodiment, whether or not such an alternative embodiment is specifically referred to.

### Experiments

A set of monoclonal antibodies were developed by genetic immunisation (Genovac GmbH, Freiburg, Germany; www.genovac.com) of mice and rats with human IL-11 (GenBank accession: NM_000641.4; derived protein sequence: GenBank accession/version: NP_000632.1; SEQ ID NO: 125). Genetic immunisation is well known to those of ordinary skill in the art (Liu S et al., Hum Gene Ther. 2018 Sep. 1; 29(9): 997-1003, doi: 10.1089/hum.2018.077). Mouse monoclonal antibodies prepared were: CSI-s-6H9, CSI-s-7H3-E4, and CSI-s-9B9 (“9B9”). Rat monoclonal antibodies prepared were: CSJ-A-s-2B11 (“2B11”), CSJ-A-s-6F4 (“6F4”), CSJ-A-s-10D10 (“10D10”), CSJ-A-s-10F9 (“1DF9”), CSJ-B-s-1E2-A5 (“1E2-A5”), CSJ-B-3F6 (also referred to herein as “CSJ-B-s-3F6”) (“3F6”), CSJ-B-s-3G8 (“3G8”), and CSJ-B-s-4B2 (“4B2”). Monoclonal antibodies were sequenced using standard commercial sequencing service (Genovac GmbH, Germany; www.genovac.com).

Antibodies were then tested using:
ELISA;
Biacore (Surface Plasmon Resonance; SPR) (Abzena Limited, Cambridge, UK—www.abzena.com; Cytiva—www.cytivalifesciences.com; Global Life Sciences Solutions Operations UK Limited, Sheffield, UK);
PK studies (WuXi Biologics, China);
In vitro neutralization; and
In vivo efficacy tests.

In particular:
Experiment 1 shows antigen binding by each of mAbs 3F6, 9B9, 2B11, 10D10, and 3G8.
Experiment 2 shows K_{D} values for binding of human IL-11 by each of mAbs 3F6, 9B9, 2B11, 10D10, and 3G8. The resulting Kovalues are significantly better than (i.e. are lower than) that obtained previously (WO2018109174 and WO2019/238882).
Experiment 3 details binding of mouse IL-11 (mIL-11) by various mAbs.
Experiment 4 details binding of rat antibody CSJ-A-s-2B11 to human and mouse IL11.
Experiment 5 shows the binding of mAb CSJ-B-3F6 to cynomolgus IL11.
Experiment 6 shows further enhanced K_{D} values (i.e. lower values) for the various humanized mAbs based on the 3F6 VH and VL sequences.
Experiment 7 shows neutralization of human IL-11 by the various humanized mAbs.
Experiment 8 shows excellent half-life (t_{1/2}) periods for mAbs 3F6, 2611, 10D10, and 3G8.
Experiment 9 shows in vivo efficacy of IL-11 mAb clones 3F6, 9B9, 2B11 and 3G8 in preventing acetaminophen-induced liver injury (AILI).
Experiment 10 shows in vivo efficacy of IL-11 mAb clones 3F6, 9B9, 10D10 and 10F9 in preventing acetaminophen-induced liver injury (AILI).

### Experiment 1 —Direct Antigen Binding ELISA

MAB218 (anti-human-IL-11 reference antibody—R&D Systems, Inc., MN, USA—www.rndsystems.com) was used to assess binding to human IL-11.

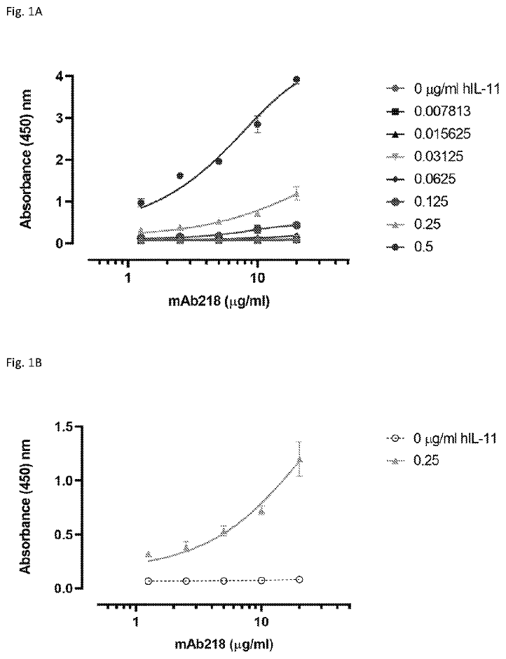

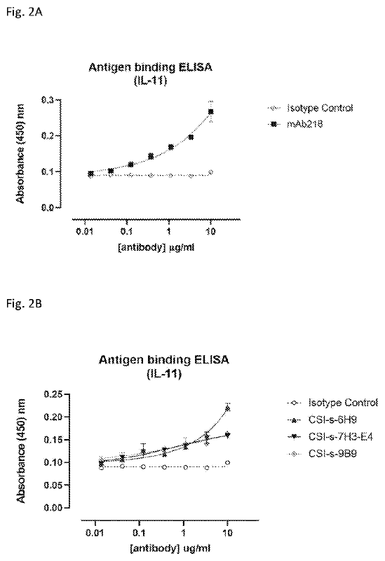

Various concentrations of human IL-11 coated on ELISA plates were tested (see FIG. 1A and FIG. 1B). 0.25 μg/ml IL-11 was selected to coat ELISA plates for antigen binding ELISAs.

The following anti-human-IL-11 mouse monoclonal antibodies were then tested: CSI-s-6H9, CSI-s-7H3-E4, and CSI-s-9B9 (“989”).

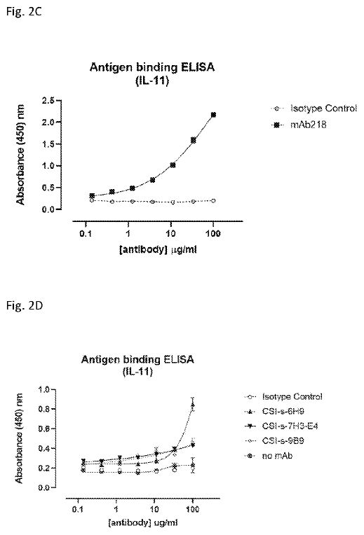

Results are shown in FIGS. 2A and 2B (tested from 10 μg/ml) and FIGS. 2C and 2D (tested from 10 μg/ml). Positive binders were identified as: CSI-s-6H9, CSI-s-7H3-E4, and CSI-s-9B9 (“9B9”).

### Method

1. Coat Nunc Immuno MaxiSorp plate and incubate overnight at 4DegC (with Genscript human IL-11 #Z03108 at 0.25 g/mL in PBS, 100 μL per well).
2. Wash plates 3× PBS-Tween-20 (250 L/well. Blot to remove excess liquid.
3. Block with 100 μL 1% BSA/PBS for 1 hour at room temperature.
4. Wash plates as in step 2.
5. Prepare 1 in 3 serial dilutions of test antibodies starting at 10 or 100 g/mL in 1% BSA/PBS. Add 100 μl/well, cover plates and incubate for 1 hour at RT.
6. Wash plates as in step 2.
7. Add 100 μL of diluted anti-mouse IgG Fc Fragment HRP conjugate (Sigma A0168, 1:5000 dilution) to each well. Cover and incubate plate for 1 hour at RT.
8. Wash plates as in step 2.
9. Add 100 μL of TMB substrate (at RT) to each well and incubate at room temperature (watch plates for colour change)
10. Stop the reaction by adding 100 μL of 3M HCL to each well. Read absorbance at 450 nm (plate reader)

Further antigen binding ELISA testing against human IL-11 was then performed for a number of rat anti-IL-11 mAbs. Monoclonal antibodies tested were: CSJ-A-s-2Bii (“2811”), CSJ-A-s-6F4 (“6F4”), CSJ-A-s-10D10 (“10D10”), CSJ-A-s-10F9 (“10F9”), and CSJ-B-s-1E2-A5 (“1E2-A5”).

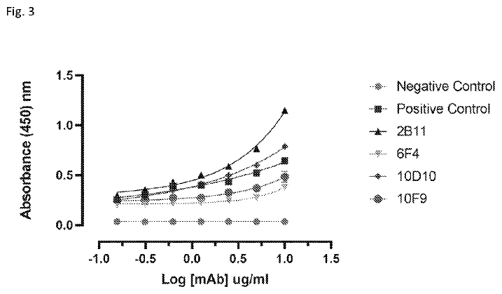

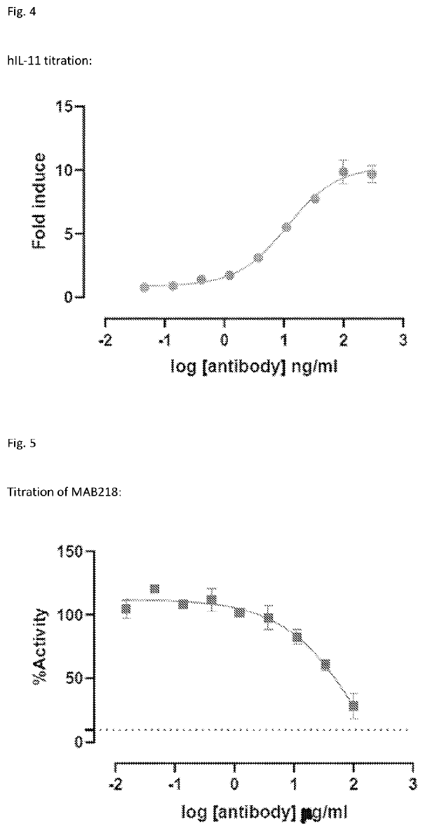

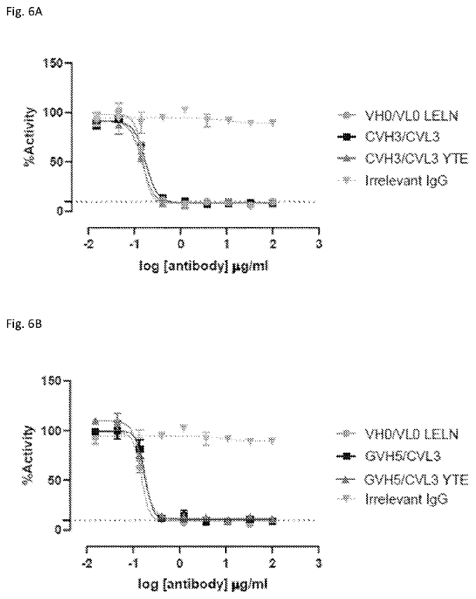

A range of binding was observed for Rat IL-11 mAbs to human IL-11. Results are shown in FIG. 3.

Negative and positive controls showed expected levels of binding. Positive binders include 2B11 and 10D10.

### Method

Using the previous ELISA methodology (above):
1. Genscript Human IL-11 #Z03108 was coated at 0.25 g/mL in PBS, 100lL per well
2. Rat anti-IL-11 mAbs were prepared using a 1 in 3 serial dilution in 1% BSA/PBS from 10 μg/mL to 0 g/mL.
3. Goat anti-rat IgG Fc HRP antibody (Abcam PLC, Cambridge, UK, Catalog #: ab97090; www.abcam.com)was used in place of the anti-mouse IgG Fv Framework HRP conjugate.
   hIL-11 TITRATION: (FIG. 4)
Full dose-response curve observed.
EC50 of IL-11: 11.4 ng/mL

### Biacore Testing

In order to assess the binding of antibodies to IL-11, SPR (surface plasmon resonance) testing was performed. Experiments were undertaken to determine human IL-11, mouse IL-11 and cyno IL-11 binding.

### Experiment 2—Human IL-11 Binding

Conditions used:
Instrument: Biacore T200 running Biacore T200 Evaluation Software V2.0.1
Running buffer: HBS-P+1 mg/mL BSA
Chip: Series S Anti-mouse antibody capture kit
Analysis Temp: 25 DegC
Ligand: Purified antibody loaded to −160 RU diluted in running buffer to 1.5 μg/mL
Analyte: Human IL-11 (Genscript, NJ, USA; Cat. No. Z0310B)
Flow rate: 30 l/min
Association: 210 s
Dissociation: 900 s
Dilution Range: 6 point three-fold dilution from 90 nM to 0.37 nM (hl-11)
Regeneration: 10 mM glycine pH 1.7
Analysis: 1:1 binding with double reference subtraction For mouse and rat antibody capture, Cytiva capture kit (Cat. No. BR100838) was used.

### Results are as Follows:

TABLE 44

| | | ka | kd | K_{D} | | Chi² |
|---|---|---|---|---|---|---|
| Ligand | Analyte | (1/Ms) | (1/s) | (M) | R_{MAX} | (RU²) |
| CSI-s-9B9 | hIL-11 | 2.58E+06 | 3.73E−04 | 1.45E−10 | 16.5 | 0.242 |
| CSJ-A-s-2B11 | hIL-11 | 2.75E+06 | 6.49E−05 | 2.38E−11 | 15.4 | 0.109 |
| CSJ-A-s-10D10 | hIL-11 | 3.71E+06 | 1.57E−03 | 4.24E−10 | 8.3 | 0.0265 |
| CSJ-B-3F6 | hIL-11 | 8.92E+06 | 1.42E−04 | 1.59E−11 | 26.1 | 0.196 |
| CSJ-B-s-3G8 | hIL-11 | 1.97E+07 | 3.44E−04 | 1.74E−11 | 9.9 | 0.0134 |

kₐ: Association rate constant
k_{d}: Dissociation rate constant
K_{D}: Equilibrium dissociation constant (kd/ka)
R_{MAX}: Analyte binding level
RU: Resonance units
Note:
K_{D} (the equilibrium dissociation constant) between an antibody and the antigen - it is a ratio of K_{off}/Kₒₙ (i.e. the dissociation rate constant k_{d}/the association rate constant kₐ). Thus, lower K_{D} values indicate higher antibody affinity for the antigen, i.e. higher binding affinity.

Experiment 3—Mouse IL-11 (mIL11) Binding
Conditions used:
Instrument: Biacore T200 running Biacore T200 Evaluation Software V2.0.1
Running buffer: HBS-P+1 mg/mL BSA
Chip: Series S Anti-mouse antibody capture kit
Analysis Temp: 25 DegC
Ligand: Purified antibody loaded to ~160 RU diluted in running buffer to 1.5 μg/mL
Analyte: Mouse IL11 (Genscript, NJ, USA; Cat. No. Z02769)
Flow rate: 40 μl/min
Association: 240 s (10D10: 210 s)
Dissociation: 900 s
Dilution Range: 8 point three-fold dilution from 90 nM to 0.04 nM (miL11) (10D10: 7 point three-fold
dilution from 90 nM to 0.12 nM)
Regeneration: 10 mM glycine pH 1.7
Analysis: 1:1 binding with double reference subtraction

### Results are as Follows:

TABLE 45

| | | ka | kd | K_{D} | | Chi² |
|---|---|---|---|---|---|---|
| Ligand | Analyte | (1/Ms) | (1/s) | (M) | R_{MAX} | (RU²) |
| CSI-s-9B9 | mIL11 | 3.36E+06 | 1.27E−02 | 3.77E−09 | 18.6 | 0.219 |
| CSJ-A-s-2B11 | mIL11 | | | 2.82E−08 | 15.5 | 0.0466 |
| CSJ-A-s-10D10 | mIL11 | 5.42E+06 | 4.74E−03 | 8.75E−10 | 7.7 | 0.0234 |
| CSJ-B-3F6 | mIL11 | 2.15E+07 | 1.29E−03 | 5.97E−11 | 26.3 | 0.188 |
| CSJ-B-s-3G8 | mIL11 | 2.40E+07 | 1.20E−03 | 5.02E−11 | 27.9 | 0.082 |

### IL-11 Binding Summary

TABLE 46

| Ligand | Species | hIL11 KD (M) | mIL11 KD (M) | Relative binding (mIL11:hIL11) |
|---|---|---|---|---|
| CSI-s-9B9 | Mouse IgG1/k | 1.45E-10 | 3.77E-09 | 26.00 |
| CSJ-A-s-2B11 | Rat IgG2b/k | 2.138E-11 | 2.82E-08 | 1318.99 |
| CSJ-A-s-10D10 | Rat IgG2b/k | 4.24E-10 | 8.75E-10 | 2.06 |
| CSJ-B-3F6 | Rat IgG2b/k&l | 1.59E-11 | 5.97E-11 | 3.75 |
| CSJ-B-s-3G8 | Rat IgG2b/k | 1.74E-11 | 1.59E-10 | 9.14 |

### Alternative Embodiment

In order to assess the binding of antibodies binding to human IL-11 antigen in an alternative embodiment, multi-cycle kinetic analysis was performed on purified material. Kinetic experiments were performed at 25DegC on a Biacore 8K running Biacore 8K Control software V3.0.12.15655 and Biacore Insight Evaluation software V3.0.12.15655 (Cytiva, Uppsala, Sweden). HBS-P+(Cytiva, Marlborough, USA), supplemented with 0.1% BSA w/v (Sigma, Dorset, UK) was used as running buffer as well as for ligand and analyte dilutions. Purified antibodies were diluted to 0.5 μg/mL in running buffer and at the start of each cycle, loaded onto Fc2, on channels 1-7 of a Protein A capture sensor chip (GE Healthcare, Little Chalfont, UK). Antibodies were captured at a flow rate of 10 μL/min to give an immobilisation level (RL) of ~75 RU. The surface was then allowed to stabilise. Multi-cycle kinetic data was obtained using human IL-11 antigen as the analyte injected at a flow rate of 50 μL/min to minimise any potential mass transfer effects. An eight point, two-fold dilution of human IL-11 was prepared in running buffer. For each concentration, the association phases were monitored for 210 seconds, and the dissociation phase was measured for 900 seconds. Regeneration of the sensor chip surface was conducted between cycles using two injections of 10 mM Glycine-HCl, pH 1.5. Multiple repeats of a blank and of human IL-11 were programmed into the kinetic run in order to check the stability of both the surface and analyte over the kinetic cycles. The signal from the reference channel Fc1 (no IgG captured) was subtracted from that of Fc2, for each channel to correct for bulk effect and differences in non-specific binding to a reference surface. The signal from each IgG blank run (IgG captured but no antigen) was subtracted to correct for differences in surface stability. The double referenced sensorgrams were fitted with the Langmuir (1:1) binding model where the closeness of fit of the data to the model is evaluated using the Chi square value which describes the deviation between the experimental and fitted (observed and expected) curves. The fitting algorithm seeks to minimize the Chi square value. A summary of the kinetic constants determined from the 1:1 model fitted curves is shown in this application. Binding was compared to the chimeric antibody.

### Experiment 4—I11 SCK (Single Cell Kinetics) Steady State Analysis (Scaled to 2B11)

In order to assess the binding of rat antibody CSJ-A-s-2B11 to human and mouse I111, the following experiment was undertaken. mAb418 (R&D systems Cat. No. MAB418-SP) was used as a positive control for mIL11 binding.

### Conditions Used:

Instrument: Biacore T200 running Biacore T200 Evaluation Software V2.0.1
Running buffer: HBS-P+ 1 mg/mL BSA
Chip: Series S Anti-mouse antibody capture kit
Analysis Temp: 25 DegC
Ligand: Purified antibody loaded to ~160 RU diluted in running buffer to 2 g/mL
Analyte: Human IL11 (Genscript Cat. No. Z03108) or mouse IL11 (Genscript Cat. No. Z03052)
Association: 120 s
Dissociation: 1500 s
Dilution Range: 4 point three-fold dilution from 0.37 nM to 10 nM (hlL11) or two, four point three-fold
dilutions from 10 to 270 nM or 0.37 nM to 10 nM) (mIL11)
Regeneration: 10 mM glycine pH 1.7
Analysis: Assuming 1:1 binding with double reference subtraction

Binding to hIL11 is observed for 2B11. No apparent binding to hIL11 by mAb418 is observed under the same conditions.

Binding to mIL11 is observed for mAb418. Binding of 2B11 to mIL11 is also observed although the 1:1 model does not fit particularly well (biphasic dissociation). In order to investigate this further a steady state model was applied (assumes a 1:1 interaction).

TABLE 47

| | | Ks | Kd | K_{D} | | | |
|---|---|---|---|---|---|---|---|
| Ligand | Analyte | (1/Ms) | (1/s) | (M) | R_{MAX} | Chi²(RU²) | Analysis |
| 2B11 | hIL11 | 3.11E+06 | 6.62E−05 | 2.13E−11 | 15.9 | 0.0105 | Kinetic |
| | mIL11 | — | — | 2.82E−08* | 15.5 | 0.0466 | Steady state |
| mAB418 | hIL11 | — | — | — | — | — | Kinetic |
| | mIL11 | 2.02E+05 | 5.45E−04 | 2.70E−09 | 33.9 | 0.482 | Kinetic |

*Assumes a 1 to 1 interaction

Binding to mIL11 is observed for 2B11 although the binding affinity (assuming a 1:1 interaction) is approximately 1000 times weaker than it is to hIL11, i.e. 2B11 binding affinity for hIL11 is approximately 1000 times greater than for mIL11.

### Experiment 5—Cynomologus IL11 Binding

In order to assess the binding of mAb CSJ-B-3F6 to cynomolgus I11, the following MCK (multi cycle kinetics) study was undertaken.

### Conditions Used:

Instrument: Biacore T200 running Biacore T200 Evaluation Software
Running buffer: HBS-P+1 mg/mL BSA
Chip: Series S Anti-mouse antibody capture kit (previously shown to stably bind to Rat IgGs) Analysis Temp: 25 DegC
Ligand: Purified antibody loaded to ~160 RU diluted in running buffer to 1.5 mg/mL
Analyte: Human IL11 (Genscript Cat. No. Z03108) or cynomolgus IL11 (Sino Biological Cat. No. 90925-CNCE)
Flow rate: 50 μl/min
Association: 210 s
Dissociation: 900 s
Dilution Range: 8 point two-fold dilution from 30 nM to 0.23 nM
Regeneration: 10 mM glycine pH 1.7
Analysis: 1:1 binding with double reference subtraction

TABLE 48

| | | ka | kd | K_{D} | | Chi² | Relative |
|---|---|---|---|---|---|---|---|
| Ligand | Analyte | (1/Ms) | (1/s) | (M) | R_{MAX} | (RU²) | binding |
| CSJ-B-3F6 | Hu IL11 | 1.24E+07 | 1.78E−04 | 1.44E−11 | 27.6 | 0.18 | 6.52 |
| CSJ-B-3F6 | Cy IL11 | 1.24E+07 | 1.17E−03 | 9.39E−11 | 26.4 | 0.03 | |

The binding of 3F6 to cynomolgus IL11 is approximately 6.5-fold weaker than binding to human IL11.

Humanized mAbs

The VH and VL sequences of mAb3F6 (VH0 and VL0) and the humanised variants (i.e. VH0/VL0, CVH3 (D54E)/CVL3, CVH4 (D54E)/CVL3, GVH5 (D54E)/CVL3, and GVH5 (D54E)/GVL5; see above—SEQ ID NOs: 91-100) were codon optimised for CH0 (Chinese Hamster Ovary) cells and synthesised (GeneArt, Regensburg, Germany) with flanking restriction enzyme sites for cloning into the pANT expression vector system (Abzena, UK) for human IgG1 (G1m3 allotype) heavy chain and kappa light chain. The VH regions were cloned between the Mlu I and Hind Ill restriction sites, and the VL regions were cloned between the Pte I and BamH I restriction sites. All constructs were confirmed by sequencing.

Small Scale Transient Expression of mAb3F6 Chimeric and Humanized Antibodies

The various expression vectors were transiently transfected into CHO cells using a TranslT Pro (Mirus Bio LLC; www.miruisbio.com) transfection method. Transfections were performed in 5 mL cultures grown in CHOgro media (Mirus Bio LLC) supplemented with 8 mM Glutamine in spin tubes and incubated with shaking at 32 DegC, 8% CO2. Culture supernatants were harvested on day 7 post-transfection and antibody concentrations were measured on an Octet QK 384 using Protein A biosensors (Molecular Devices, Wokingham, Berkshire, UK), using an IgG1 antibody as standard.

Experiment 6—Multi-Cycle Kinetic (MCK) Analysis of Chimeric and Humanised mAbs

In order to assess the binding of the humanised variants to human IL-11 antigen, multi-cycle kinetic analysis was performed on purified material. Kinetic experiments were performed at 25 DegC on a Biacore 8K running Biacore 8K Control software V3.0.12.15655 and Biacore Insight Evaluation software V3.0.12.15655 (Cytiva, Uppsala, Sweden).

HBS-P+(Cytiva, Marlborough, USA), supplemented with 0.1% BSA w/v (Sigma, Dorset, UK) was used as running buffer as well as for ligand and analyte dilutions. Purified antibodies were diluted to 0.5 Vg/m L in running buffer and at the start of each cycle, loaded onto Fc2, on channels 1-7 of a Protein A capture sensor chip (GE Healthcare, Little Chalfont, UK). Antibodies were captured at a flow rate of 10 L/min to give an immobilisation level (RL) of ~75 RU. The surface was then allowed to stabilise.

Multi-cycle kinetic data was obtained using human IL-11 antigen as the analyte injected at a flow rate of 50 μL/min to minimise any potential mass transfer effects. An eight point, twofold dilution range from 4 nM to 0.03125 nM of human IL-11 was prepared in running buffer. For each concentration, the association phases were monitored for 210 seconds, and the dissociation phase was measured for 900 seconds. Regeneration of the sensor chip surface was conducted between cycles using two injections of 10 m M Glycine-HCl, pH 1.5. Multiple repeats of a blank and of human IL-11 were programmed into the kinetic run in order to check the stability of both the surface and analyte over the kinetic cycles (data not shown).

The signal from the reference channel Fc1 (no IgG captured) was subtracted from that of Fc2, for each channel to correct for bulk effect and differences in non-specific binding to a reference surface. The signal from each IgG blank run (IgG captured but no antigen) was subtracted to correct for differences in surface stability. The double referenced sensorgrams were fitted with the Langmuir (1:1) binding model where the closeness of fit of the data to the model is evaluated using the Chi square value which describes the deviation between the experimental and fitted (observed and expected) curves. The fitting algorithm seeks to minimize the Chi square value.

TABLE 49

| | Kₐ | K_{d} | K_{D} | Relative | | Chi² |
|---|---|---|---|---|---|---|
| Antibody | (1/Ms) | (1/s) | (M) | K_{D} | R_{MAX} | (RU²) |
| VH0/VL0 (FWR4: LELN) | 4.80E+07 | 3.64E−04 | 7.58E−12 | — | 19.1 | 0.02 |
| CVH3 (D54E)/CVL3 | 3.82E+07 | 1.91E−04 | 5.01E−12 | 0.66 | 15.5 | 0.05 |
| CVH3 (D54E)/ | 3.48E+07 | 4.93E−05 | 1.42E−12 | 0.19 | 20.2 | 0.01 |
| CVL3 + YTE | | | | | | |
| CVH4 (D54E)/CVL3 | 4.24E+07 | 3.17E−04 | 7.48E−12 | 0.99 | 16.7 | 0.01 |
| CVH4 (D54E)/ | 9.07E+07 | 3.03E−04 | 3.34E−12 | 0.44 | 16.5 | 0.09 |
| CVL3 + YTE | | | | | | |
| GVH5 (D54E)/CVL3 | 2.24E+07 | 4.06E−04 | 1.81E−11 | 2.39 | 19.5 | 0.01 |
| GVH5 (D54E)/ | 3.82E+07 | 3.77E−04 | 9.89E−12 | 1.30 | 14.6 | 0.03 |
| CVL3 + YTE | | | | | | |
| GVH5 (D54E)/GVL5 | 2.10E+07 | 2.03E−03 | 9.66E−11 | 12.73 | 20.1 | 0.07 |
| GVH5 (D54E)/ | 3.61E+07 | 2.86E−03 | 7.92E−11 | 10.44 | 17.2 | 0.04 |
| GVL5 + YTE | | | | | | |

The relative Ko was calculated by dividing the Ko of the humanised variant by that of the VH0/VL0 assayed in the same experiment.

### Experiment 7—Human IL-11 Neutralization

To assess the inhibitory biological activity of the various mAbs, IgGs were assessed using the STAT3 Reporter (Luc)—HEK293 Cell line (Puromycin) (BPS Bioscience, CA, USA; www.bpsbioscience.com; Catalog #79800-P).

The STAT3 Reporter (Luc)-HEK293 cell line is designed for monitoring the STAT3 signal transduction pathway. It contains a firefly luciferase gene, driven by STAT3 response elements, located upstream of the minimal TATA promoter. After activation by cytokines, endogenous STAT3 binds to the DNA response elements, inducing transcription of the luciferase reporter gene.

### Assay Method (Supplier's Recommendation Followed)

Cells seeded at 30,000 cells/well (96w clear bottom white plate) for overnight. (incubate at 37 DegC/5% CO₂)
Next day (after 24 hours incubation), media was removed and replaced with 50 μl fresh media. Cells were then incubated for 1 hour at 37 DegC/5% CO₂.
Test samples and controls were diluted and mixed with rhlL-11 and incubated for 30 mins at 37° C.
Both samples and cytokine were made up 4× assay concentration.
After 1 hour incubation 50 μl of test sample—rhIL11 mixture was added to the cells and incubated for 24 hours at 37 DegC.
Control wells:
   Untreated and unstimulated (=no test sample and no cytokine)
   No cell wells.
After 24-hour incubation detection reagent was added and read luminescence (SpectraMax i3× plate reader)
Data analysis: Excel and GraphPad Prism.

### Results

Cytokine titration: Fold induction was determined by comparing the values against the mean value for untreated control cells.

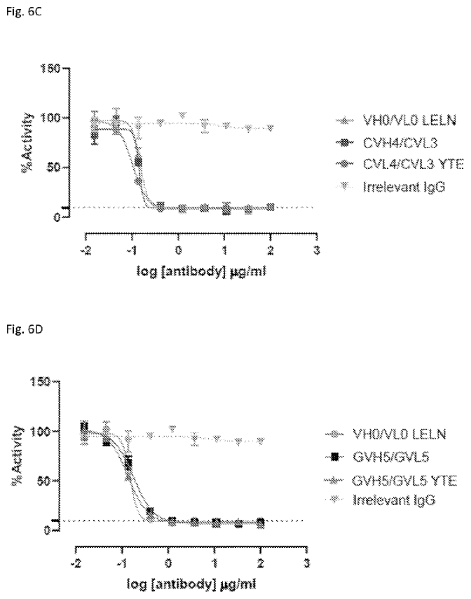

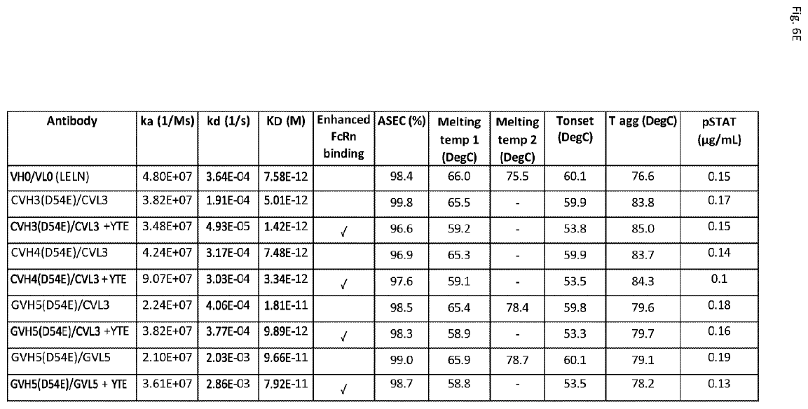

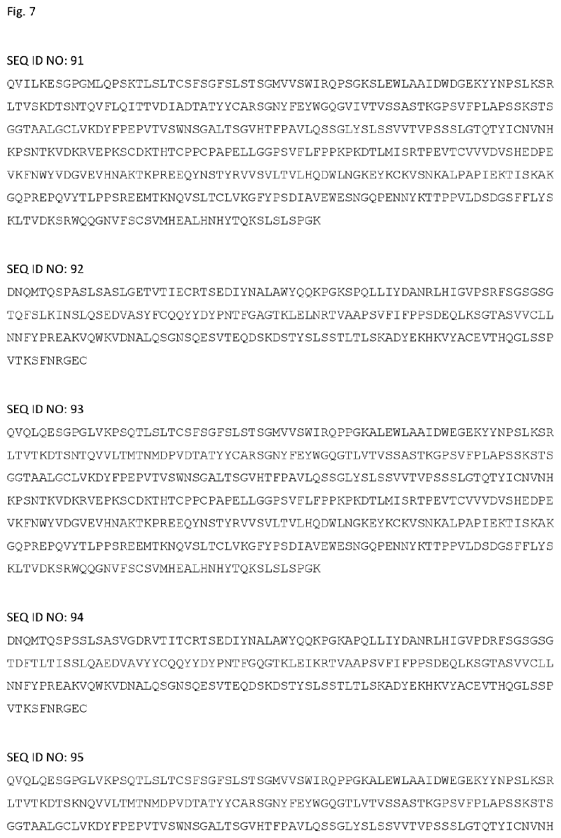

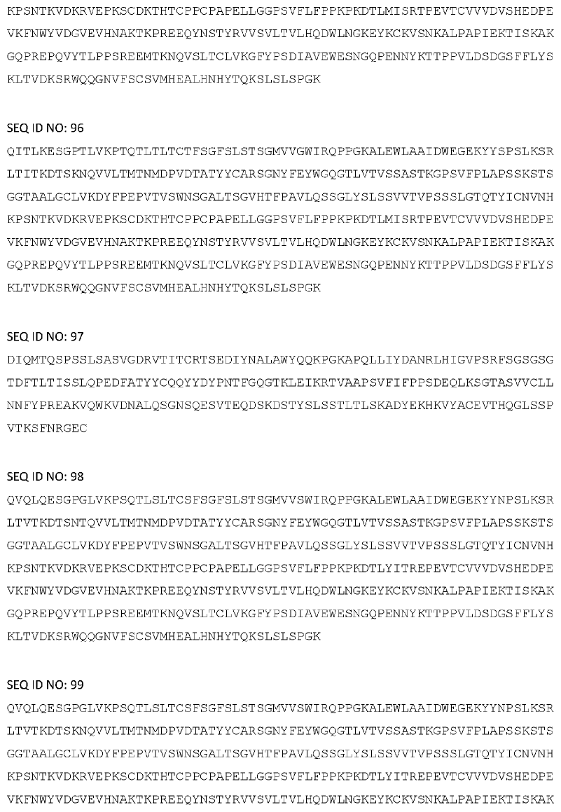

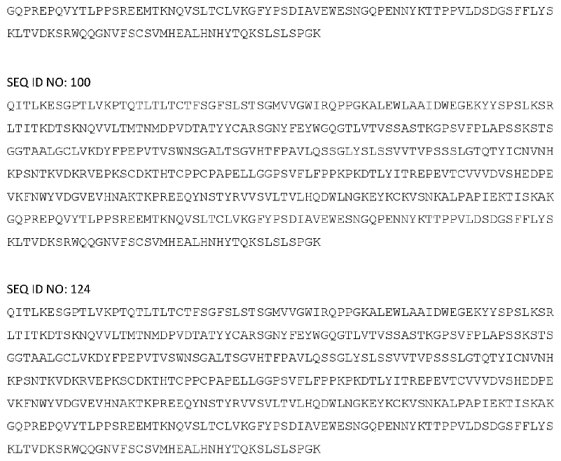

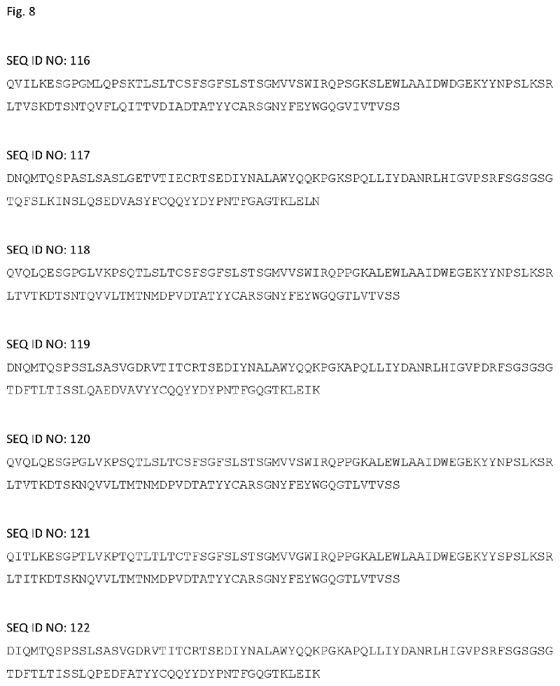

Results for VH0/VL0, CVH3 (D54E)/CVL3, CVH3 (D54E)/CVL3+YTE, CVH4 (D54E)/CVL3, CVH4 (D54E)/CVL3+YTE, GVH5 (D54E)/CVL3, GVH5 (D54E)/CVL3+YTE, GVH5 (D54E)/GVL5, and GVH5 (D54E)/GVL5+YTE are shown in FIGS. 6A-6D.

### Experiment 8—Preclinical PKStudy

A preclinical PK (pharmacokinetic) study was undertaken for candidate antibodies.

The study was performed with male SD (Sprague Dawley) rats, with an administered dose of 5 mg/kg of the antibody, formulated at 2 mg/ml in PBS buffer (7.4), clear solution. An ELISA method for measuring antibody concentration in rat serum was used. The lower limit of quantification (LLOQ) was 1000 ng/mL and the upper limit of quantification (ULOQ) was 16000 ng/mL concentration. For the ELISA assay, the capture antigen was human IL-11 (Genscript, catalogue #: Z03108). For 2B11, 10D10 and 3F6, the HRP was goat anti-rat IgG HRP (Biolegend, catalogue #: 405405). For 3G8, the HRP was Peroxidase AffiniPure Goat Anti-Rat IgG, Fcγ fragment specific (Jackson ImmunoResearch Laboratories Inc., Pennsylvania USA; catalogue #: 112-035-071; www.jacksonimmuno.com). Washing buffer was 0.1% PBST. Coating buffer was PBS (phosphate buffered saline). Blocking buffer was 3% BSA (bovine serum albumin) in 0.1% PBST (phosphate buffered saline with Tween 20 (RTM)). Assay buffer was 1% BSA in 0.1% PBST. TMB (3,3′,5,5′ tetramethylbenzidine) was TMB substrate kit (SeraCare, Milford, MA, USA—www.seracare.com; catalogue #: 5120-0077). The stop solution was 2N H₂SO₄. Three repeats of the experiment were performed, and half-life (T₁;2) in hours determined, with a mean, SD (standard deviation) and CV (coefficient of variation) being calculated.

The plate reader used was a SpectraMax M5e/M5/Plus 384 (Molecular Devices) which had an assay range of 16000-1000 ng/mL. The HRP dilution factor was 1:1000, and the coating solution working concentration was 0.25 μg/mL.

### Assay Procedure:

1. Add 100 μL coating solution to the 96 well microplate, seal, and incubate overnight at 2-8 DegC before use.
2. Wash plate 3 times with wash buffer. (300 μL/well).
3. Block the plate by adding 300 μL of Blocking Buffer to each well. Seal and incubate for 2 hours at RT (room temperature) without shaking.
4. Repeat step 2.
5. Add 100 μL of standard and samples into each well. Seal and incubate 120±10 min at RT, shaking at 450RPM.
6. Repeat step 2.
7. Add 100 μL of HRP solution into each well. Seal and incubate for 90±10 min at RT, shaking at 450RPM.
8. Repeat step 2.
9. Add 100 μL of TMB into each well. Incubate for 5-30 minutes at RT.
10. Add 100 μL Stop Solution into each well.
11. Read plate using a plate reader within 30 min in 450 nm and 630 nm.

Results are as follows:

TABLE 50

| Monoclonal | Mean T_{1/2} (h) | SD | CV (%) |
|---|---|---|---|
| CSJ-A-s-2B11 | 195 | 5.56 | 2.85 |
| CSJ-A-s-10D10 | 161 | 6.73 | 4.18 |
| CSJ-B-s-3F6 | 232 | 29.1 | 12.5 |
| CSJ-B-s-3G8 | 196 | 17.6 | 8.98 |

### Experiment 9—In Vivo Testing of the Efficacy of IL-11 mAb Clones in Preventing Acetaminophen-Induced Liver Injury (AILI)

Antibodies tested: CSJ-A-s-2B11, CSJ-B-s-3F6, CSJ-B-s-3G8, CSJ-B-s-4B2, CSI-s-9B9. Negative control: 11E10 (mouse monoclonal shigatoxin-2 antibody, IgG1, Genovac)
Mice: C57BL/6nTac (InVivos, Singapore)
Colorimetric kits:
   Alanine Transaminase (ALT) Activity Assay kit (ab105134, Abcam, Cambridge, UK)
   Aspartate Aminotransferase (AST) Activity Assay kit (abl05135, Abcam, Cambridge, UK)

### Chemicals:

0.9% Sodium Chloride (Saline; SIN 9481, B.Braun Medical Ltd., UK; www.bbraun.co.uk) Acetaminophen (APAP; A3035, Sigma)

### Study Protocols:

Mouse models of Acetaminophen (APAP) poisoning

Prior to APAP administration, 10-11-week-old male mice were fasted overnight (16 hours). Mice were administered 20 mg/kg of either CSJ-A-s-2B11, CSJ-B-3F6, CSJ-B-s-3G8, CSJ-B-s-4B2 CSP-s-9B9, CSP-s-8C6 or IgG isotype control (11E10) by intraperitoneal injection at the start of the fasting period.

Following overnight fasting, mice were given 400 mg/kg dose of APAP (APAP stock: 17.5 mg/ml in saline) by intraperitoneal administration and euthanized at 24 hours post APAP.

### Colorimetric Assays

The concentrations of ALT or AST in mouse serum were measured using ALT or AST Activity Assay Kit according to the manufacturer's protocol. ALT/AST concentrations in the serum of control mice was treated as maximal inhibition (100%), while ALT/AST concentrations in the serum of APAP-injured mice receiving 11E10 (20 mg/kg) constituted minimum inhibition (0%).

### Statistical Analysis

Statistical analyses were performed using GraphPad Prism software (version 8). Statistical significance between experimental groups were analysed by one-way ANOVA as indicated and corrected for multiple testing according to Dunnett's (with APAP+11E10 designated as the control group). The criterion for statistical significance was P<0.05.

### Results:

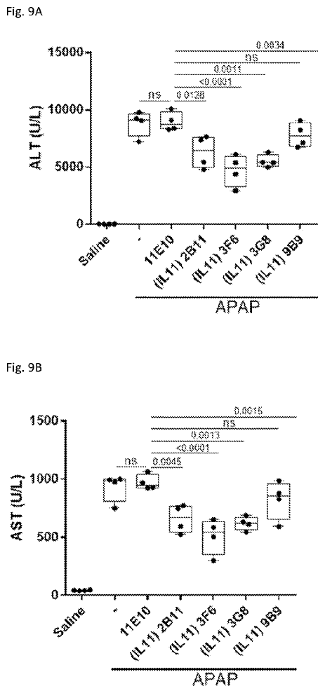

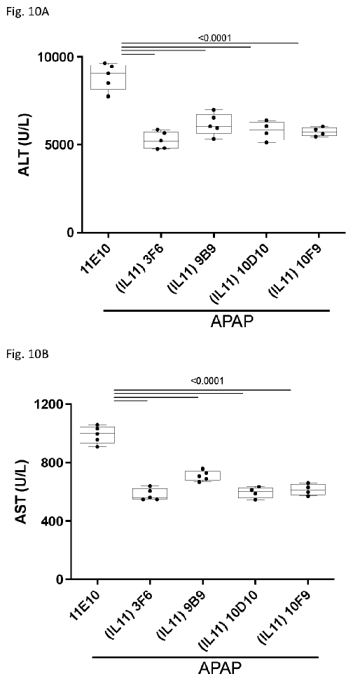

Results are shown in FIGS. 9A and 9B.

TABLE 51
Percentage (%) reduction as compared to APAP + 11E10 group.

| Assay | (IL11) 2B11 | (IL11) 3F6 | (IL11) 3G8 | (IL11) 9B9 |
|---|---|---|---|---|
| ALT | 29.6 | 47.5 | 38.4 | 13.2 |
| AST | 32 | 47.3 | 38.7 | 15.4 |
| Average | 30.8 | 47.4 | 36.6 | 14.3 |

As can be seen, from the Figures and Table 51, the various mAbs effect a significant reduction as compared to the control group (APAP+11E10).

### Experiment 10—In Vivo Testing of the Efficacy of IL-11 mAb Clones in Preventing Acetaminophen-Induced Livery Injury (AILI)

Antibodies tested: CSI-s-9B9, CSJ-B-s-3F6, CSJ-A-s-10D10, CSJ-A-s-10F9
Negative control: 11E10 (mouse monoclonal shigatoxin-2 antibody, IgG1, Genovac)
Mice: C57BL/6nTac (InVivos, Singapore)

### Colorimetric Kits:

Alanine Transaminase (ALT) Activity Assay kit (ab105134, Abcam, Cambridge, UK)
Aspartate Aminotransferase (AST) Activity Assay kit (ab105135, Abcam, Cambridge, UK)

### Chemicals:

0.9% Sodium Chloride (Saline; SIN 9481, B.Braun Medical Ltd., UK; www.bbraun.co.uk)
Acetaminophen (APAP; A3035, Sigma)

### Study Protocol:

### Mouse Models of Acetaminophen Poisoning

Prior to APAP administration, 15-week-old male mice were fasted overnight (16 hours). Mice were administered 20 mg/kg of either CSI-s-9B9, CSJ-B-s-3F6, CSJ-A-s-10D10, CSJ-A-s-10F9, CSP-s-4D4, CSP-s-5H8, CSP-s-8C6, or IgG isotype control (11E10) by intraperitoneal injection at the start of the fasting period. Following overnight fasting, mice were given 400 mg/kg dose of APAP (APAP stock: 17.5 mg/ml in saline) by intraperitoneal administration and euthanized at 24 hours post APAP.

### Colorimetric Assays

The concentrations of ALT or AST in mouse serum were measured using ALT or AST Activity Assay Kit according to the manufacturer's protocol. We considered ALT/AST concentrations in the serum of control mice as maximal inhibition (100%), while ALT/AST concentrations in the serum of APAP-injured mice receiving 11E10 (20 mg/kg) constituted minimum inhibition (0%).

### Statistical Analysis

Statistical analyses were performed using GraphPad Prism software (version 8). Statistical significance between experimental groups were analysed by one-way ANOVA as indicated and corrected for multiple testing according to Dunnett's (with APAP+11E10 designated as the control group). The criterion for statistical significance was P<0.05.

### Results:

Results are shown in FIGS. 10A and 10B.

TABLE 52
Percentage (%) reduction as compared to APAP + 11E10 group.

| Assay | (IL11) 3F6 | (IL11) 9B9 | (IL11) 10D10 | (IL11) 10F9 |
|---|---|---|---|---|
| ALT | 40.7 | 30.5 | 34.6 | 35.3 |
| AST | 41.4 | 28.3 | 39.8 | 37.9 |
| Average | 41.01 | 29.4 | 37.2 | 36.6 |

As can be seen, from the Figures and Table 52, the various mAbs effect a significant reduction as compared to the control group (APAP+11E10).

### Sequencing

Sequencing was performed by Genovac GmbH (Germany; www.genovac.com). Total RNA was isolated from freshly thawed hybridoma cells following the technical manual of Macherey-Nagel (Macherey-Nagel GmbH & Co. KG, Germany; www.mn-net.com) NucleoSpin (RTM) RNA kit. Total RNA was then reverse transcribed into cDNA using SMART RACE technology (Takara Bio USA, Inc.; www.takarabio.com). The antibody fragments of VH and VL were amplified with a universal 5′ anchor primer and isotype-specific 3′ primers. Amplified antibody fragments were gel-purified and blunt-end cloned into an appropriate vector. No less than five vectors with insert were sequenced with gene-specific primers for each fragment. The sequences of different clones were aligned and the consensus sequence of these clones was IMGT/V-Quest analysed (www.imgt.org; Brochet, X., et al., Nucl. Acids Res, 36, W503-508 (2008), PMID: 18503082, LIGM: 344, doi: 10.1093/nar/gkn316; Giudicelli, V. et al., Cold Spring Harb Protoc. 2011 Jun. 1; (2011), PMID: 21632778, LIGM: 388, doi: 10.1101/pdb.prot5633) and the signal peptide and variable region sequence is provided.

### Epitope Manning

Epitope mapping of monoclonal antibodies was performed by CovalX (CovaIX AG, Zurich, Switzerland; www.covalx.com) as detailed below.

### Samples:

The following monoclonal antibodies were used:
IL-11_Human (GenScript, NJ, USA; www.genscript.com) cat. No: Z03108 lot #B60011811: Lyophilized; 1 mg
CSI-s-9B9: PBS; 2 mg/ml; 200 μL
CSJ-A-s-2B11: PBS; 2 mg/ml; 200 μL
CSJ-A-s-10D10: PBS; 2 mg/ml; 200 iL
CSJ-B-3F6: PBS; 2 mg/ml; 200 μL
CSJ-B-s-3G8: PBS; 2 mg/ml; 200 μL

### A. High-Mass MALDI Mass Spectrometry

In a first step, a high-mass MALDI MS analysis was performed on each sample (IL-11_Human, CSI-s-9B9, CSJ-A-s-2B11, CSJ-A-s-10D10, CSJ-B-3F6 and CSJ-B-s-3G8) to verify their integrity and aggregation level.

### 1. Materials & Methods

### 1.1. Instrumentation

For the integrity/aggregation test, the measurements were performed using an Autoflex II MALDI ToF/ToF mass spectrometer (Bruker) equipped with the CovalX HM4 interaction module.

### 1.2. Sample Preparation

IL-11_Human sample was first dissolved to reach a concentration of 1 mg/ml.

TABLE 53

| | Sample initial | Sample final | |
|---|---|---|---|
| | Powder | Volume | Concentration |
| IL-11_Human | 1000 μg | 1000 μl | 1 mg/ml |

Then, 20 μl of each protein sample (IL-11_Human, CSI-s-9B9, CSJ-A-s-2B11, CSJ-A-s-10D10, CSJB-3F6 and CSJ-B-s-3G8) were pipetted to prepare 8 dilutions with final volume 10 μl. These 8 dilutions were prepared to obtain the following expected concentrations:

TABLE 54

| | IL-11_Human | | | CSI-s-9B9 | | | CSJ-A-s-2B11 | | |
|---|---|---|---|---|---|---|---|---|---|
| Dilution | Volume | Conc. | | Volume | Conc. | | Volume | Conc. | |
| 1 | 10 μl | 1 | mg/ml | 10 μl | 1 | mg/ml | 10 μl | 1 | mg/ml |
| ½ | 10 μl | 500 | μg/ml | 10 μl | 500 | μg/ml | 10 μl | 500 | μg/ml |
| ¼ | 10 μl | 250 | μg/ml | 10 μl | 250 | μg/ml | 10 μl | 250 | μg/ml |
| ⅛ | 10 μl | 125 | μg/ml | 10 μl | 125 | μg/ml | 10 μl | 125 | μg/ml |
| 1/16 | 10 μl | 62.50 | μg/ml | 10 μl | 62.50 | μg/ml | 10 μl | 62.50 | μg/ml |
| 1/32 | 10 μl | 31.25 | μg/ml | 10 μl | 31.25 | μg/ml | 10 μl | 31.25 | μg/ml |
| 1/64 | 10 μl | 15.63 | μg/ml | 10 μl | 15.63 | μg/ml | 10 μl | 15.63 | μg/ml |
| 1/128 | 10 μl | 7.81 | μg/ml | 10 μl | 7.81 | μg/ml | 10 μl | 7.81 | μg/ml |

TABLE 55

| | CSJ-A-s-10D10 | | | CSJ-B-3F6 | | | CSJ-B-s-3G8 | | |
|---|---|---|---|---|---|---|---|---|---|
| Dilution | Volume | Conc. | | Volume | Conc. | | Volume | Conc. | |
| 1 | 10 μl | 1 | mg/ml | 10 μl | 1 | mg/ml | 10 μl | 1 | mg/ml |
| ½ | 10 μl | 500 | μg/ml | 10 μl | 500 | μg/ml | 10 μl | 500 | μg/ml |
| ¼ | 10 μl | 250 | μg/ml | 10 μl | 250 | μg/ml | 10 μl | 250 | μg/ml |
| ⅛ | 10 μl | 125 | μg/ml | 10 μl | 125 | μg/ml | 10 μl | 125 | μg/ml |
| 1/16 | 10 μl | 62.50 | μg/ml | 10 μl | 62.50 | μg/ml | 10 μl | 62.50 | μg/ml |
| 1/32 | 10 μl | 31.25 | μg/ml | 10 μl | 31.25 | μg/ml | 10 μl | 31.25 | μg/ml |
| 1/64 | 10 μl | 15.63 | μg/ml | 10 μl | 15.63 | μg/ml | 10 μl | 15.63 | μg/ml |
| 1/128 | 10 μl | 7.81 | μg/ml | 10 μl | 7.81 | μg/ml | 10 μl | 7.81 | μg/ml |

The diluted samples were then prepared for High-Mass MALDI MS analysis.

### 1.3 High-Mass MALDI MS Analysis

High-Mass MALDI analysis was performed on control and cross-link samples

### 2. Results

### 2.1 IL-11_Human

### Control Experiments

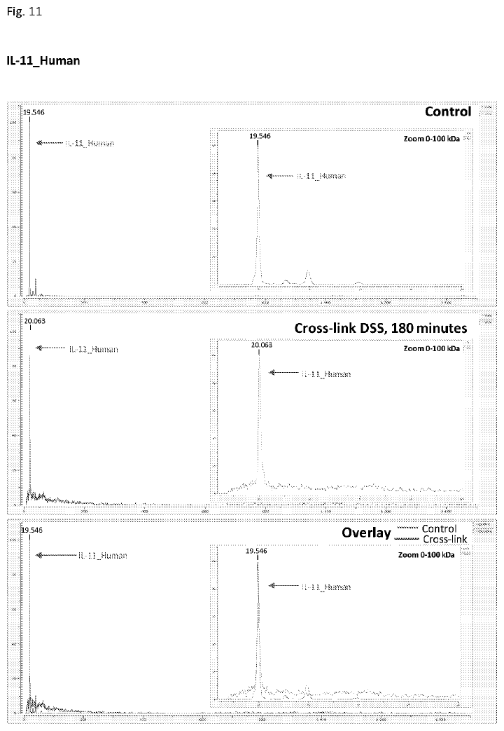

In these experiments, one main peak was detected for every dilution from 1 to 1/64 with MH+=19.546 kDa. (FIG. 11, Control)

TABLE 56

| Observed Molecular Weight (kDa) | |
|---|---|
| 19.546 | IL-11_Human |

### Cross-Link Experiments

In these experiments, one main peak was detected for every dilution from 1 to 1/64 with MH+=20.063 kDa. (FIG. 11, Cross-link)

FIG. 11: [IL-11_Human]=1.6 pM, dilution 1/32. Total Volume: 10 μl. Cross-link: DSS (disuccinimidyl suberate), 180 minutes incubation time.

TABLE 57

| Observed Molecular Weight (kDa) | |
|---|---|
| 20.063 | IL-11_Human |

Non-covalent complexes were not detected in the higher mass range (FIG. 11, Overlay).

### 2.2 CSI-s-9B9

### Control Experiments

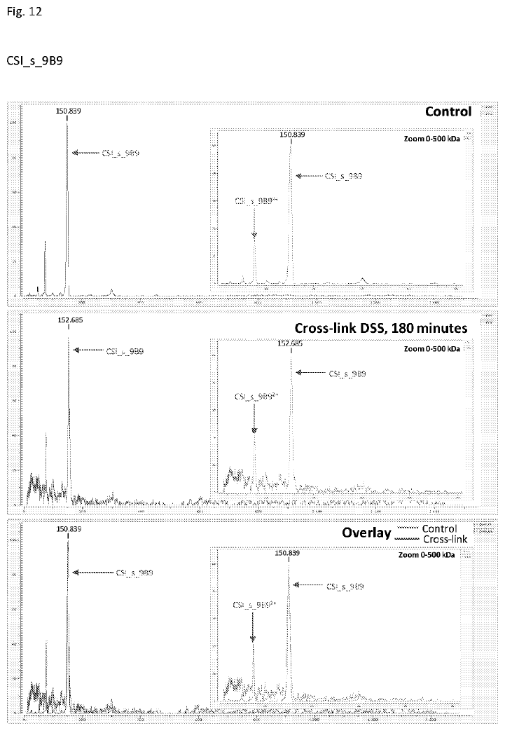

In these experiments, one main peak was detected for every dilution from 1to 1/32 with MH+=150.839 kDa. (FIG. 12, Control)

TABLE 58

| Observed Molecular Weight (kDa) | |
|---|---|
| 150.839 | CSI-s-9B9 |

### Cross-Link Experiments

In these experiments, one main peak was detected for every dilution from 1to 1/32 with MH+=152.685 kDa. (FIG. 12, Cross-link)

FIG. 12: [C5|_s_9B9]=0.41 pM, dilution 1/16. Total Volume: 10 μl. Cross-link: DSS, 180 minutes incubation time.

TABLE 59

| Observed Molecular Weight (kDa) | |
|---|---|
| 152.685 | CSI-s-9B9 |

Non-covalent complexes were not detected in the higher mass range. (FIG. 12, Overlay)

### 2.3 CSJ-A-s-2B11

### Control Experiments

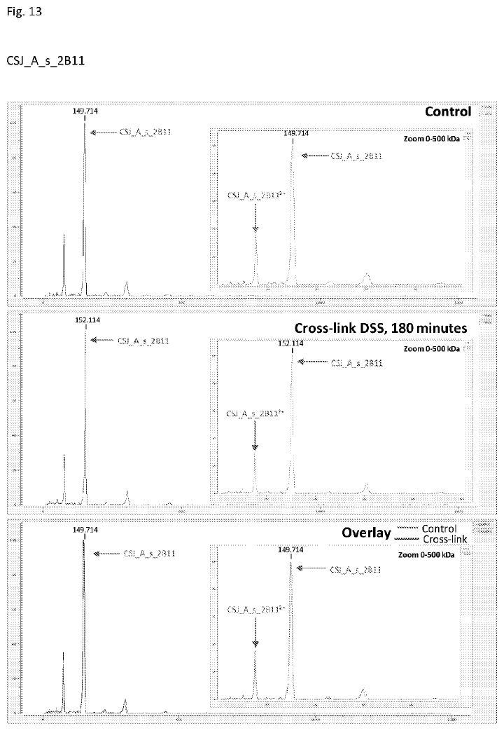

In these experiments, one main peak was detected for every dilution from 1to 1/64 with MH+=149.714 kDa. (FIG. 13, Control).

TABLE 60

| Observed Molecular Weight (kDa) | |
|---|---|
| 149.714 | CSJ-A-s-2B11 |

### Cross-Link Experiments

In these experiments, one main peak was detected for every dilution from 1to 1/32 with MH+=152.685 kDa. (FIG. 13, Cross-link).

FIG. 13: [CSJ_A_s_2B11]=0.84 μM, dilution ⅛. Total Volume: 10 μl. Cross-link: DSS, 180 minutes incubation time.

TABLE 61

| Observed Molecular Weight (kDa) | |
|---|---|
| 152.114 | CSJ-A-s-2B11 |

Non-covalent complexes were not detected in the higher mass range. (FIG. 13, Overlay).

### 2.4 CSJ-A-s-10D10

### Control Experiments

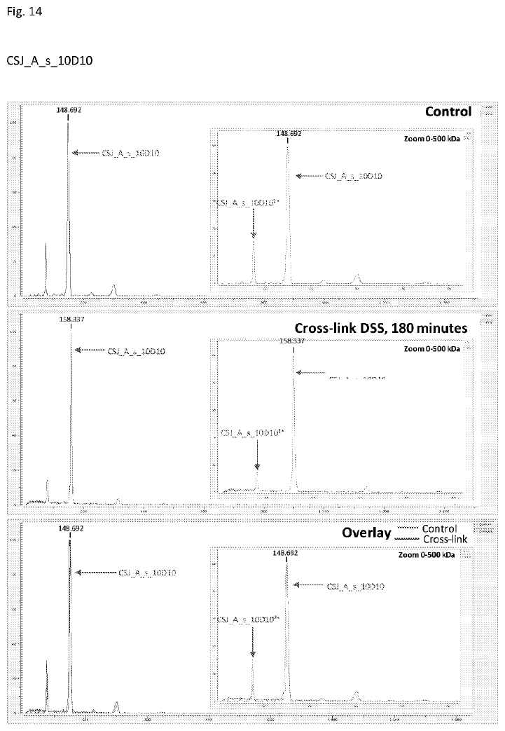

In these experiments, one main peak was detected for every dilution from 1 to 1/64 with MH+=148.692 kDa. (FIG. 14, Control)

TABLE 62

| Observed Molecular Weight (kDa) | |
|---|---|
| 148.692 | CSJ-A-s-10D10 |

### Cross-Link Experiments

In these experiments, one main peak was detected for every dilution from 1to 1/16 with MH+=158.337 kDa. (FIG. 14, Cross-link).

FIG. 14: [CSJ_A_s_10D10]=1.68ltM, dilution ¼. Total Volume: 10 μl. Cross-link: DSS, 180 minutes incubation time.

TABLE 63

| Observed Molecular Weight (kDa) | |
|---|---|
| 158.337 | CSJ-A-s-10D10 |

Non-covalent complexes were not detected in the higher mass range. (FIG. 14, Overlay)

### 2.5 CSJ-B-3F6

### Control Experiments

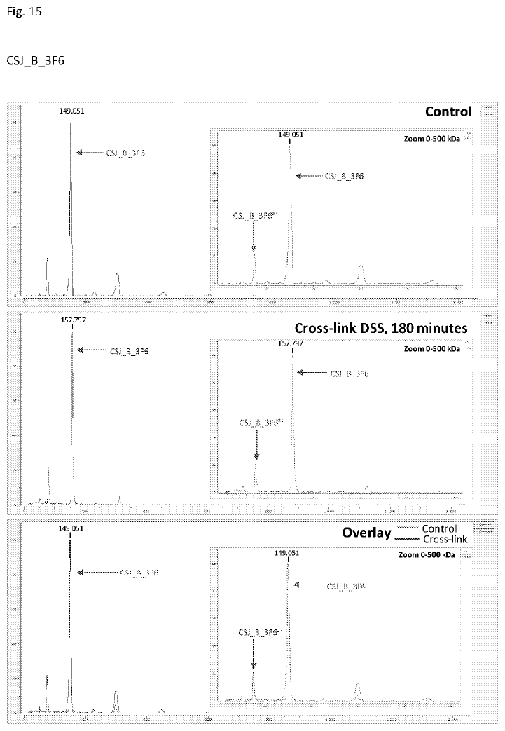

In these experiments, one main peak was detected for every dilution from 1 to 1/64 with MH+=149.051 kDa. (FIG. 15, Control)

TABLE 64

| Observed Molecular Weight (kDa) | |
|---|---|
| 149.051 | CSJ-B-3F6 |

### Cross-Link Experiments

In these experiments, one main peak was detected for every dilution from 1to 1/32 with MH+=157.797 kDa. (FIG. 15, Cross-link)

FIG. 15: [CSJ_B_3F6]=1.67 pM, dilution ¼. Total Volume: 10 I. Cross-link: DSS, 180 minutes incubation time.

TABLE 65

| Observed Molecular Weight (kDa) | |
|---|---|
| 157.797 | CSJ-B-3F6 |

Non-covalent complexes were no detected in the higher mass range. (FIG. 15, Overlay)

### 2.6 CSJ-B-s-3G8

### Control Experiments

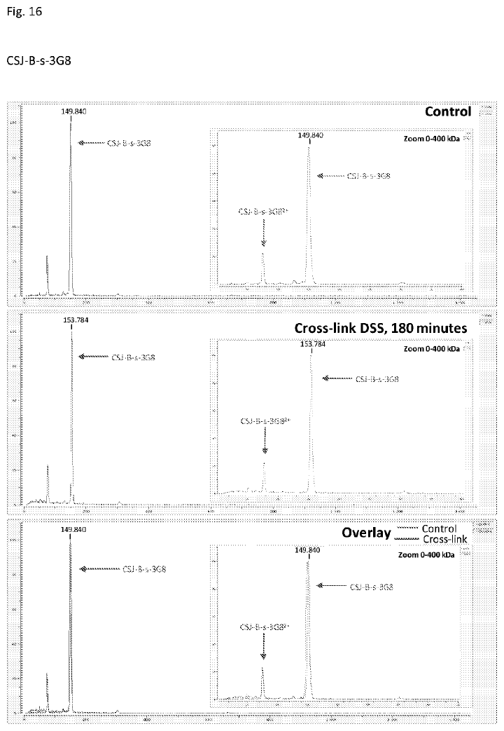

In these experiments, one main peak was detected for every dilution from 1to 1/64 with MH+=149.840 kDa. (FIG. 16, Control)

TABLE 66

| Observed Molecular Weight (kDa) | |
|---|---|
| 149.840 | CSJ-B-s-3G8 |

### Cross-Link Experiments

In these experiments, one main peak was detected for every dilution from 1to 1/32 with MH+=153.784 kDa. (FIG. 16, Cross-link)

FIG. 16: [CSJ-B-s-3G8]=0.4 M, dilution 1/16. Total Volume: 10 μl. Cross-link: DSS, 180 minutes incubation time

TABLE 67

| Observed Molecular Weight (kDa) | |
|---|---|
| 153.784 | CSJ-B-s-3G8 |

Non-covalent complexes were not detected in the higher mass range. (FIG. 16, Overlay)

### 3. Conclusion Aggregation Test

High-Mass MALDI mass spectrometry and chemical cross-linking did not result in detection of any non-covalent aggregate of the antibodies (CSI-s-9B9, CSJ-A-s-2B11, CSJ-A-s-10D10, CSJ-B-3F6 and CSJ-B-s-3G8) or multimers of IL-11_Human.

### B. Characterization of IL-11_Human/Antibodies Protein Complexes

### 1. Material, Method

### 1.1. Instrumentation

For the characterization of IL-11 Human/CSI-s-9B9, IL-11_Human/CSJ-A-s-2B11, IL-11 Human/CSJ-A-s-10D10, IL-11_Human/CSJ-B-3F6 and IL-11_Human/CSJ-B-s-3G8 complexes, measurements were performed using an Autoflex II MALDI ToF mass spectrometer (Bruker, MA, USA; www.bruker.com) equipped with the CovalX HM4 interaction module.

### 1.2. Sample Preparation

### Control Experiments

The mixture IL-11_Human/CSI-s-9B9, IL-11_Human/CSJ-A-s-2B11, IL-11_Human/CSJ-A-s-10D10, IL-11_Human/CSJ-B-3F6 and IL-11_Human/CSJ-B-s-3G8 were prepared with the following concentrations:

TABLE 68

| | | | | | IL-11_Human/ | |
|---|---|---|---|---|---|---|
| | IL-11_Human | | Antibody | | Antibody | |
| Mixture | Volume | Conc. | Volume | Conc. | Volume | Conc. |
| IL-11_Human/CSI-s-9B9 | 5 μl | 4 μM | 5 μl | 1 μM | 10 μl | 2 μM/0.5 μM |
| IL-11_Human/CSJ-A-s-2B11 | 5 μl | 4 μM | 5 μl | 2 μM | 10 μl | 2 μM/1 μM |
| IL-11_Human/CSJ-A-s-10D10 | 5 μl | 4 μM | 5 μl | 2 μM | 10 μl | 2 μM/1 μM |
| IL-11_Human/CSJ-B-3F6 | 5 μl | 4 μM | 5 μl | 2 μM | 10 μl | 2 μM/1 μM |
| IL-11_Human/CSJ-B-s-3G8 | 5 μl | 4 μM | 5 μl | 1 μM | 10 μl | 2 μM/0.5 μM |

The protein mixture was prepared for High-Mass MALDI MS analysis.

1.3. High-Mass MALDI MS analysis

The High-Mass MALDI analysis has been performed following CovalX.

### 2. Results

### 2.1. IL-11_Human/CSI-s-9B9

### Control Experiments

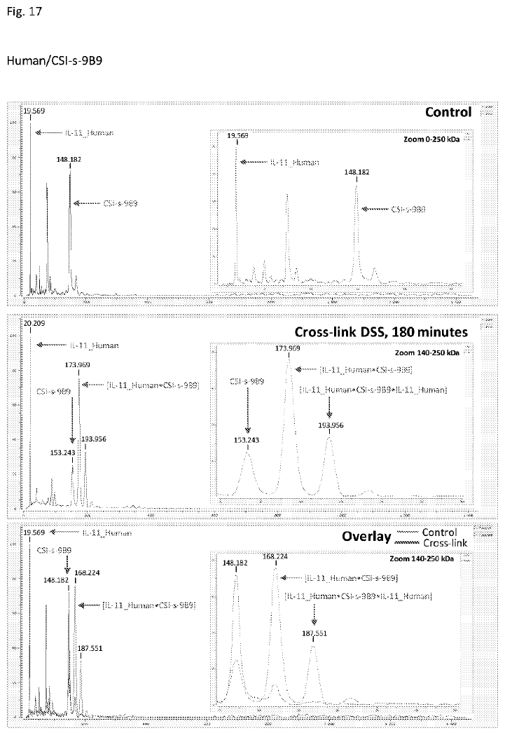

IL-11_Human and CSI-s-9B9 were detected with MH+=19.569 kDa and MH+=148.182 kDa. (FIG. 17, Control)

TABLE 69

| Observed Molecular Weight (kDa) | |
|---|---|
| 19.569 | IL-11 Human |
| 148.182 | CSI-s-9B9 |

### Cross-Link Experiments

The cross-linking experiment was completed after 180 minutes incubation time with the DSS reagent. After cross-linking, two additional peaks were detected with MH+=173.969 kDa and MH+=193.956 kDa. (FIG. 17, Cross-link)

TABLE 70

| Observed Molecular Weight (kDa) | |
|---|---|
| 173.969 | [IL-11_Human• |
| | CSI-s-9B9] |
| 193.956 | [IL-11_Human•CSI-s-9B9• |
| | IL-11_Human] |

The control and cross-link spectra were overlaid. Two non-covalent protein complexes with MH+=168.224 kDa and MH+=187.551 kDa were detected. (FIG. 17, Overlay)

TABLE 71

| Observed Molecular Weight (kDa) | |
|---|---|
| 168.224 | [IL-11_Human•CSI-s-9B9] |
| 187.551 | [IL-11_Human•CSI-s-9B9• |
| | IL-11_Human] |

FIG. 17: [IL-11_Human]=2 M, [CSI-s-9B9]=0.5 pM. Total Volume: 10 μl. Cross-link: DSS, 180 minutes incubation time.

### 2.2. IL-11_Human/CSJ-A-s-2B11

### Control Experiments

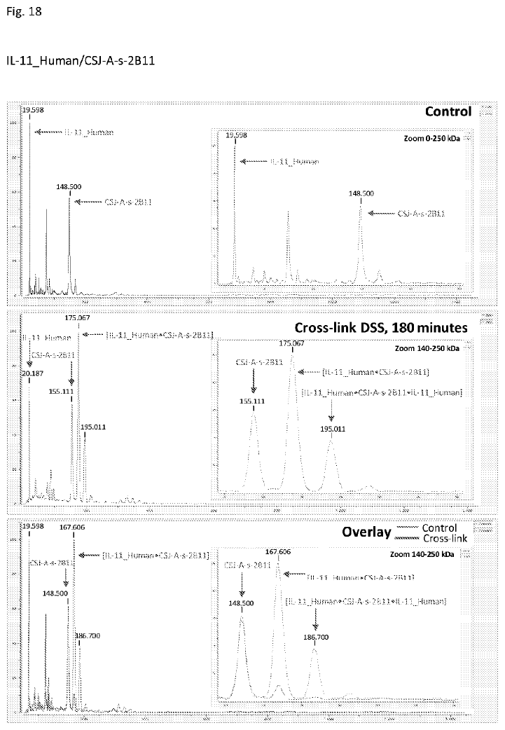

IL-11_Human and CSJ-A-s-2B11 were detected with MH+=19.598 kDa and MH+=148.500 kDa. (FIG. 18, Control).

TABLE 72

| Observed Molecular Weight (kDa) | |
|---|---|
| 19.598 | IL-11_Human |
| 148.500 | CSJ-A-s-2B11 |

### Cross-Link Experiments

The cross-linking experiment was completed after 180 minutes incubation time with the DSS reagent. After cross-linking, two additional peaks were detected with MH+=175.067 kDa and MH+=195.011 kDa. (FIG. 18, Cross-link)

TABLE 73

| Observed Molecular Weight (kDa) | |
|---|---|
| 175.067 | [IL-11_Human•CSJ-A-s- |
| | 2B11] |
| 195.011 | [IL-11_Human•CSJ-A-s-2B11• |
| | IL-11_Human] |

The control and cross-link spectra were overlaid. Two non-covalent protein complexes with MH+=167.606 kDa and MH+=186.700 kDa were detected. (FIG. 18, Overlay).

TABLE 74

| Observed Molecular Weight (kDa) | |
|---|---|
| 167.606 | [IL-11_Human•CSJ-A-s-2B11] |
| 186.700 | [IL-11_Human•CSJ-A-s-2B11•IL- |
| | 11_Human] |

FIG. 18: [IL-11_Human]=2 pM, [CSJ-A-s-2B11]=1 M. Total Volume: 10 ii. Cross-link: DSS, 180 minutes incubation time.

### 2.3. IL-11_Human/CSJ-A-s-10D10

### Control Experiments

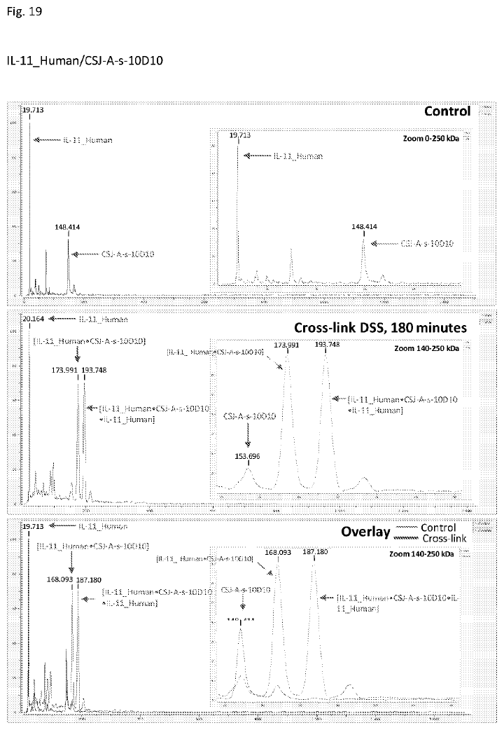

IL-11_Human and CSJ-A-s-10D10 were detected with MH+=19.713 kDa and MH+=148.414 kDa. (FIG. 19, Control)

TABLE 75

| Observed Molecular Weight (kDa) | |
|---|---|
| 19.713 | IL-11_Human |
| 148.414 | CSJ-A-s-10D10 |

### Cross-Link Experiments

The cross-linking experiment was completed after 180 minutes incubation time with the DSS reagent. After cross-linking, two additional peaks were detected with MH+=173.991 kDa and MH+=193.748 kDa. (FIG. 19, Cross-link)

TABLE 76

| Observed Molecular Weight (kDa) | |
|---|---|
| 173.991 | [IL-11_Human•CSJ-A-s-10D10] |
| 193.748 | [IL-11_Human•CSJ-A-s-10D10•IL-11_Human] |

The control and cross-link spectra were overlaid. Two non-covalent protein complexes with MH+=168.093 kDa and MH+=187.180 kDa were detecte3d. (FIG. 19, Overlay)

TABLE 77

| Observed Molecular Weight (kDa) | |
|---|---|
| 168.093 | [IL-11_Human•CSJ-A-s-10D10] |
| 187.180 | [IL-11_Human•CSJ-A-s-10D10•IL-11_Human] |

FIG. 19: [IL-11_Human]=2 pM, [CSJ-A-s-10D10]=1 kM. Total Volume: 10 μl. Cross-link: DSS, 180 minutes incubation time.

### 2.4. IL-11_Human/CSJ-B-3F6

### Control Experiments

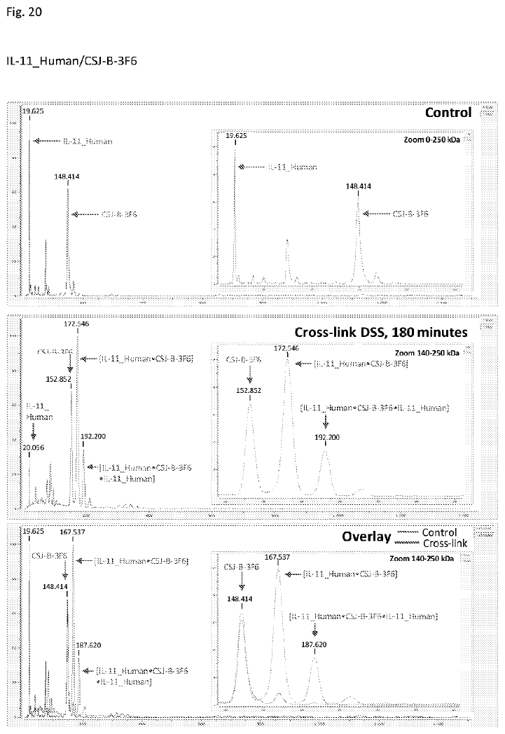

For this experiment, IL-11_Human and CSJ-B-3F6 were detected with MH+=19.625 kDa and MH+=148.414 kDa. (FIG. 20, Control)

TABLE 78

| Observed Molecular Weight (kDa) | |
|---|---|
| 19.713 | IL-11_Human |
| 148.414 | CSJ-B-3F6 |

### Cross-Link Experiments

The cross-linking experiment was completed after 180 minutes incubation time with the DSS reagent. After cross-linking, two additional peaks were detected with MH+=172.546 kDa and MH+=192.200 kDa. (FIG. 20, Cross-link)

TABLE 79

| Observed Molecular Weight (kDa) | |
|---|---|
| 172.546 | [IL-11_Human•CSJ-B-3F6] |
| 192.200 | [IL-11_Human•CSJ-B-3F6•IL- |
| | 11_Human] |

The control and cross-link spectra were overlaid. Two non-covalent protein complexes with MH+=167.537 kDa and MH+=187.620 kDa were detected. (FIG. 20, Overlay)

TABLE 80

| Observed Molecular Weight (kDa) | |
|---|---|
| 167.537 | [IL-11_Human•CSJ-B-3F6] |
| 187.620 | [IL-11_Human•CSJ-B-3F6•IL- |
| | 11_Human] |

FIG. 20: [IL-11_Human]=2 pM, [CSJ-B-3F6]=1 pM. Total Volume: 10 μl. Cross-link: DSS, 180 minutes incubation time.

### 2.5. IL-11_Human/CSJ_B_s_3G8

### Control Experiments

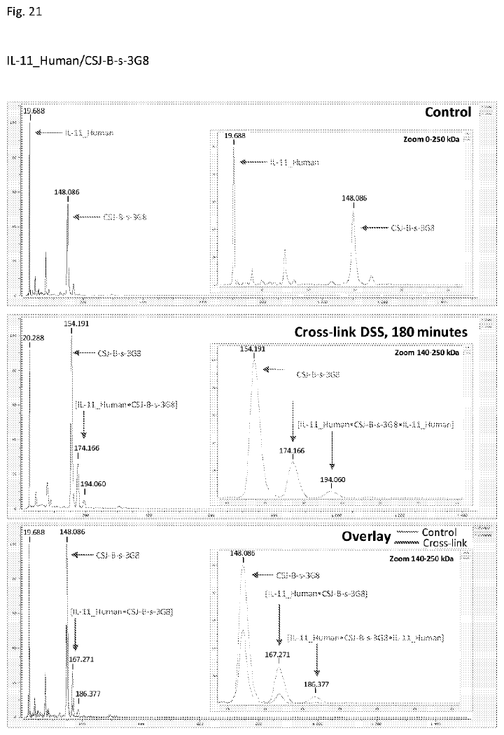

For this experiment, IL-11_Human and CSJ_B_s_3G8 were detected with MH+=19.688 kDa and MH+=148.086 kDa. (FIG. 21, Control)

TABLE 81

| Observed Molecular Weight (kDa) | |
|---|---|
| 19.688 | IL-11_Human |
| 148.086 | CSJ_B_s_3G8 |

### Cross-Link Experiments

The cross-linking experiment was completed after 180 minutes incubation time with the DSS reagent. After cross-linking, two additional peaks were detected with MH+=174.166 kDa and MH+=194.060 kDa. (FIG. 21, Cross-link)

TABLE 82

| Observed Molecular Weight (kDa) | |
|---|---|
| 174.166 | [IL-11_Human•CSJ_B_s_3G8] |
| 194.060 | [IL-11_Human•CSJ_B_s_3G8•IL-11_Human] |

The control and cross-link spectra were overlaid. Two non-covalent protein complexes with MH+=167.271 kDa and MH+=186.377 kDa were detected. (FIG. 21, Overlay)

TABLE 83

| Observed Molecular Weight (kDa) | |
|---|---|
| 167.271 | [IL-11_Human•CSJ_B_s_3G8] |
| 186.377 | [IL-11_Human•CSJ_B_s_3G8•IL-11_Human] |

FIG. 21: [IL-11_Human]=2 pM, [CSJ-B-s-3G8]=0.5 pM. Total Volume: 10 ∥l. Cross-link: DSS, 180 minutes incubation time.

### C. Characterization and Peptide Mass Fingerprint of IL-11_Human.

To characterize IL-11_Human, the sample was subjected to trypsin, chymotrypsin, Asp-N, elastase and thermolysin proteolysis followed by nLC-Q-Exactive MS/MS analysis.

### 1. Material, Method

### 1.1. Instrumentation

For the characterization of IL-11_Human, a nLC Ultimate 3000-RSLC system (Thermo Scientific) in line with a Q-Exactive mass spectrometer (Thermo Scientific; Thermo Fisher Scientific Inc, MA, USA; www.thermofisher.com) was used.

1.2. Sample preparation:

Two tubes of IL-11_Human (Control, Cross-link) were prepared (0.5 mg/ml, 10 μl). One of the tubes (cross-link) was mixed with 1 iL of DSS d0/d12 (2 mg/mL; DMF (dimethylformamide)) before 180 minutes incubation time at room temperature. Then, both tubes prepared were submitted to reduction/alkylation and proteolysis with five different enzymes (Trypsin, Chymotrypsin, ASP-N, Elastase and Thermolysin).

1.3. Liquid chromatography

After proteolysis, 1 μl of each peptide solution generated by proteolysis was loaded onto a nano-liquid chromatography system (Ultimate 3000-RSLC (Thermo Scientific)).

### 1.4. Mass Spectrometry: Q-Exactive MS Analysis

The nLC chromatographic system is in line with the Q-Exactive Plus mass spectrometer.

### 2. Results

2.1. Trypsin proteolysis

32 peptides were identified in the sequence of IL-11_Human (SEQ ID NO: 125), covering 94.94% of the sequence. The peptides are identified in the table below with reference to their position within SEQ ID NO: 125.

TABLE 84
Identified peptides of IL-11_Human after Trypsin proteolysis.

| Sequence | Modifications | Position Peptide |
|---|---|---|
| SEQ ID NO: 125 | | 31-47 |
| SEQ ID NO: 125 | | 37-47 |
| SEQ ID NO: 125 | | 37-54 |
| SEQ ID NO: 125 | | 48-54 |
| SEQ ID NO: 125 | | 48-61 |
| SEQ ID NO: 125 | | 55-61 |
| SEQ ID NO: 125 | | 55-63 |
| SEQ ID NO: 125 | | 55-96 |
| SEQ ID NO: 125 | | 62-96 |
| SEQ ID NO: 125 | | 62-98 |
| SEQ ID NO: 125 | | 64-96 |
| SEQ ID NO: 125 | | 64-106 |
| SEQ ID NO: 125 | | 97-106 |
| SEQ ID NO: 125 | | 99-106 |
| SEQ ID NO: 125 | | 99-112 |
| SEQ ID NO: 125 | | 107-112 |
| SEQ ID NO: 125 | | 107-113 |
| SEQ ID NO: 125 | | 113-132 |
| SEQ ID NO: 125 | | 114-132 |
| SEQ ID NO: 125 | | 114-135 |
| SEQ ID NO: 125 | | 121-132 |
| SEQ ID NO: 125 | | 121-135 |
| SEQ ID NO: 125 | | 121-138 |
| SEQ ID NO: 125 | | 133-138 |
| SEQ ID NO: 125 | | 136-146 |
| SEQ ID NO: 125 | | 139-146 |
| SEQ ID NO: 125 | | 140-146 |
| SEQ ID NO: 125 | | 147-172 |
| SEQ ID NO: 125 | | 173-190 |
| SEQ ID NO: 125 | | 173-196 |
| SEQ ID NO: 125 | | 173-198 |
| SEQ ID NO: 125 | | 191-199 |

2.2. Chymotrypsin proteolysis PP16T,

27 peptides were identified in the sequence of IL-11_Human (SEQ ID NO: 125), covering 65.15% of the sequence. The peptides are identified in the table below with reference to their position within SEQ ID NO: 125.

TABLE 85
Identified peptides of IL-11_Human after Chymotrypsin proteolysis.

| Sequence | Modifications | Position Peptide |
|---|---|---|
| SEQ ID NO: 125 | | 45-50 |
| SEQ ID NO: 125 | | 46-56 |
| SEQ ID NO: 125 | | 50-56 |
| SEQ ID NO: 125 | | 51-60 |
| SEQ ID NO: 125 | | 79-88 |
| SEQ ID NO: 125 | | 86-94 |
| SEQ ID NO: 125 | | 89-97 |
| SEQ ID NO: 125 | | 91-97 |
| SEQ ID NO: 125 | | 95-101 |
| SEQ ID NO: 125 | | 95-102 |
| SEQ ID NO: 125 | | 98-104 |
| SEQ ID NO: 125 | | 103-110 |
| SEQ ID NO: 125 | | 105-110 |
| SEQ ID NO: 125 | | 105-111 |
| SEQ ID NO: 125 | | 130-137 |
| SEQ ID NO: 125 | | 141-147 |
| SEQ ID NO: 125 | | 148-168 |
| SEQ ID NO: 125 | | 169-178 |
| SEQ ID NO: 125 | | 169-181 |
| SEQ ID NO: 125 | | 169-183 |
| SEQ ID NO: 125 | | 179-185 |
| SEQ ID NO: 125 | | 182-187 |
| SEQ ID NO: 125 | | 184-192 |
| SEQ ID NO: 125 | | 186-192 |
| SEQ ID NO: 125 | | 186-193 |
| SEQ ID NO: 125 | | 188-194 |
| SEQ ID NO: 125 | | 194-199 |

2.3. ASP-N proteolysis

11 peptides were identified in the sequence of IL-11_Human (SEQ ID NO: 125), covering 62.35% of the sequence. The peptides are identified in the table below with reference to their position within SEQ ID NO: 125.

TABLE 86
Identified peptides of IL-11_Human after ASP-N
proteolysis.

| | Modifi- | Position |
|---|---|---|
| Sequence | cations | Peptide |
| SEQ ID NO: 125 | | 34-39 |
| SEQ ID NO: 125 | | 40-51 |
| DSTVLLTRSLLADTRQLAAQLR | | 40-61 |
| SEQ ID NO: 125 | | 52-61 |
| SEQ ID NO: 125 | | 52-66 |
| SEQ ID NO: 125 | | 52-68 |
| SEQ ID NO: 125 | | 62-68 |
| SEQ ID NO: 125 | | 62-72 |
| SEQ ID NO: 125 | | 73-99 |
| SEQ ID NO: 125 | | 155-199 |
| SEQ ID NO: 125 | | 186-199 |

2.4. Elastase proteolysis

17 peptides were identified in the sequence IL-11_Human (SEQ ID NO: 125), covering 60.67% of the sequence. The peptides are identified in the table below with reference to their position within SEQ ID NO: 125.

TABLE 87
Identified peptides of IL-11_Human after Elastase proteolysis.

| Sequence | Modifications | Position Peptide |
|---|---|---|
| SEQ ID NO: 125 | | 32-43 |
| SEQ ID NO: 125 | | 50-56 |
| SEQ ID NO: 125 | | 52-58 |
| SEQ ID NO: 125 | | 61-78 |
| SEQ ID NO: 125 | | 89-97 |
| SEQ ID NO: 125 | | 103-108 |
| SEQ ID NO: 125 | | 103-111 |
| SEQ ID NO: 125 | | 106-111 |
| SEQ ID NO: 125 | | 106-114 |
| SEQ ID NO: 125 | | 109-114 |
| SEQ ID NO: 125 | | 123-129 |
| SEQ ID NO: 125 | | 148-167 |
| SEQ ID NO: 125 | | 149-167 |
| SEQ ID NO: 125 | | 168-174 |
| SEQ ID NO: 125 | | 179-185 |
| SEQ ID NO: 125 | | 182-188 |
| SEQ ID NO: 125 | | 186-192 |

### 2.5. Thermolysin Proteolysis

16 peptides were identified in the sequence of IL-11_Human (SEQ ID NO: 125). covering 66.85% of the sequence. The peptides are identified in the table below with reference to their position within SEQ ID NO: 125.

TABLE 88
Identified peptides of IL-11_Human after Thermolysin proteolysis.

| Sequence | Modifications | Position Peptide |
|---|---|---|
| SEQ ID NO: 125 | | 22-30 |
| SEQ ID NO: 125 | | 31-40 |
| SEQ ID NO: 125 | | 31-42 |
| SEQ ID NO: 125 | | 51-57 |
| SEQ ID NO: 125 | | 62-71 |
| SEQ ID NO: 125 | | 66-78 |
| SEQ ID NO: 125 | | 90-96 |
| SEQ ID NO: 125 | | 99-104 |
| SEQ ID NO: 125 | | 108-118 |
| SEQ ID NO: 125 | | 119-125 |
| SEQ ID NO: 125 | | 126-132 |
| SEQ ID NO: 125 | | 133-139 |
| SEQ ID NO: 125 | | 147-155 |
| SEQ ID NO: 125 | | 147-166 |
| SEQ ID NO: 125 | | 183-188 |
| SEQ ID NO: 125 | | 185-191 |

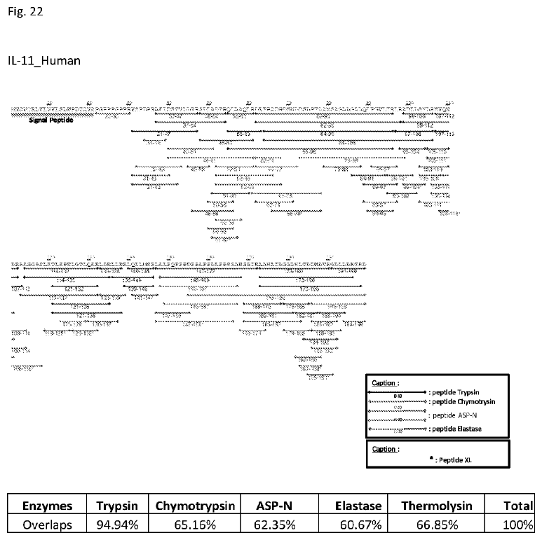

FIG. 22 is an overlap mapping of the Trypsin, Chymotrypsin, ASP-N, Elastase and Thermolysin peptides using the results obtained (above). Combining the peptides of Trypsin, Chymotrypsin, ASP-N, Elastase and Thermolysin proteolysis, 100% of the sequence is covered.

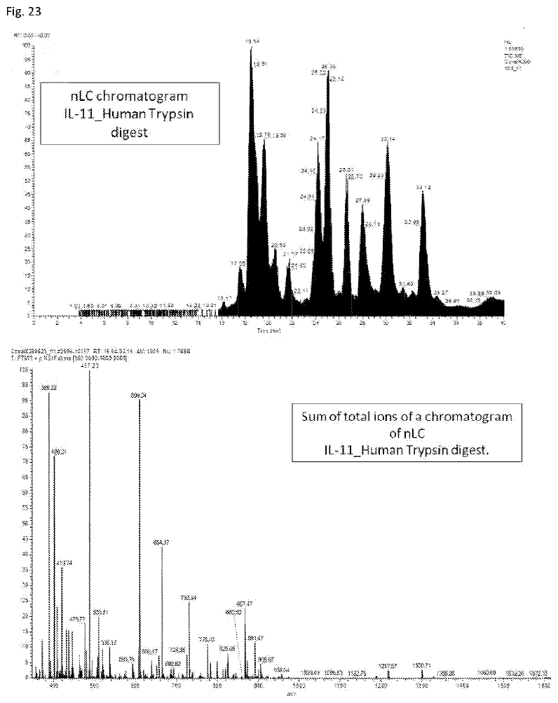

The nLC chromatogram and the total sum of the ions detected by the Q-Exactive for Trypsin digest of IL-11_Human are presented in FIG. 23.

FIG. 23: Chromatogram IL-11_Human. [IL-11_Human]=0.05 mg/mL

### D. Characterization of the Molecular Interfaces

To determine the epitope of the antibodies (CSI-s-9B9, CSJ-A-s-2B11, CSJ-A-s-10D10, CSJ-B-3F6 and CSJ-B-s-3G8) on IL-11_Human complexes with high resolution, the protein complexes were incubated with deuterated cross-linkers and subjected to multi-enzymatic cleavage. After enrichment of the cross-linked peptides, the samples were analyzed by high resolution mass spectrometry (nLC-Q-Exactive MS) and the data generated was analyzed using XQuest (Leitner, A., Walzthoeni, T. & Aebersold, R. Lysine-specific chemical cross-linking of protein complexes and identification of cross-linking sites using LC-MS/MS and the xQuest/xProphet software pipeline. Nat Protoc 9, 120-137 (2014). Doi: 10.1038/nprot.2013.168) and StavroX (Gbtze, M., Pettelkau, J., Schaks, S. et al. StavroX-A Software for Analyzing Crosslinked Products in Protein Interaction Studies. J. Am. Soc. Mass Spectrom. 23, 76-87 (2012). Doi: 10.1007/s13361-011-0261-2) software.

### 1. Material, Method

### 1.1. Instrumentation

For this analysis, nLC chromatographies in combination with Q-Exactive mass spectrometry was used as described above in section C; 1.3 and 1.4.

### 1.2. Sample Preparation:

The mixture IL-11_Human/CSI-s-9B9, IL-11_Human/CSJ-A-s-2B11, IL-11_Human/CSJ-A-s-10D10, IL-11_Human/CSJ-B-3F6 and IL-11_Human/CSJ-B-s-3G8 were prepared with the following concentrations:

TABLE 89

| | | | | | IL-11_Human/ | |
|---|---|---|---|---|---|---|
| | IL-11_Human | | Antibody | | Antibody | |
| Mixture | Volume | Conc. | Volume | Conc. | Volume | Conc. |
| IL-11_Human/CSI-s-9B9 | 10 μl | 4 μM | 10 μl | 1 μM | 20 μl | 2 μM/0.5 μM |
| IL-11_Human/CSJ-A-s-2B11 | 10 μl | 4 μM | 10 μl | 2 μM | 20 μl | 2 μM/1 μM |
| IL-11_Human/CSJ-A-s-10D10 | 10 μl | 4 μM | 10 μl | 2 μM | 20 μl | 2 μM/1 μM |
| IL-11_Human/CSJ-B-3F6 | 10 μl | 4 μM | 10 μl | 2 μM | 20 μl | 2 μM/1 μM |
| IL-11_Human/CSJ-B-s-3G8 | 10 μl | 4 μM | 10 μl | 1 μM | 20 μl | 2 μM/0.5 μM |

20 μL of each (IL-11_Human/CSI-s-9B9, IL-11_Human/CSJ-A-s-2B11, IL-11_Human/CSJ-A-s-10D10, IL-11_Human/CSJ-B-3F6 and IL-11_Human/CSJ-B-s-3G8) mixture prepared were mixed with 2 μL of DSS d0/d12 (2 mg/mL; DMF) before 180 minutes incubation time at room temperature. After incubation, the samples prepared were submitted to reduction/alkylation and proteolysis with five different enzymes (Trypsin, Chymotrypsin, ASP-N, Elastase and Thermolysin).

### 1.3. Liquid Chromatography

After proteolysis, 1 μl of each peptide solution generated by proteolysis was loaded onto a nano-liquid chromatography system (Ultimate 3000-RSLC).

### 1.4. Mass Spectrometry: Q-Exactive MS Analysis

The nLC chromatographic system is in line with the Q-Exactive mass spectrometer.

### 2. Results

### 2.1. IL-11_Human/CSI-s-9B9

After Trypsin, Chymotrypsin, ASP-N, Elastase, and Thermolysin proteolysis of the protein complex IL-11_Human/CSI-s-9B9 with deuterated d0d12, nLC-Q-Exactive MS/MS analysis detected 11 cross-linked peptides between IL-11_Human and CSI-s-9B9.

The sequences and positions of cross-links are presented in Table 90 (below).

TABLE 90
Cross-linked peptides detected between IL-11_Human (SEQ ID NO: 125) and CSI-s-9B9 VH/VK (SEQ ID NO: 20 and
SEQ ID NO: 29) complementarity determining regions (CDRs). “nAA1” and “nAA2” are the amino
acids in Protein 1 and Protein 2 respectively that are crosslinked with one another. “XLType” indicates the crosslink type.

| | Protein 1 | Protein 2 | Protein 1 | Protein 2 | | | |
|---|---|---|---|---|---|---|---|
| Enzyme | (SEQ ID NO) | (SEQ ID NO) | Position | Position | XLType | nAA1 | nAA2 |
| Thermolysin | CSI-s-9B9_VH (20) | IL-11_Human (125) | 101-108 | 19-36 | inter-protein xl | 103 | 30 |
| Chymotrypsin | CSI-s-9B9_VK (29) | IL-11_Human (125) | 12-36 | 40-44 | inter-protein xl | 24 | 41 |
| Thermolysin | CSI-s-9B9_VH (20) | IL-11_Human (125) | 51-67 | 37-42 | inter-protein xl | 58 | 41 |
| Thermolysin | CSI-s-9B9_VH (20) | IL-11_Human (125) | 51-67 | 39-44 | inter-protein xl | 58 | 41 |
| Elastase | CSI-s-9B9_VH (20) | IL-11_Human (125) | 49-66 | 40-44 | inter-protein xl | 54 | 41 |
| Chymotrypsin | CSI-s-9B9_VK (29) | IL-11_Human (125) | 16-32 | 86-97 | inter-protein xl | 26 | 95 |
| Elastase | CSI-s-9B9_VH (20) | IL-11_Human (125) | 94-104 | 98-102 | inter-protein xl | 98 | 98 |
| Elastase | CSI-s-9B9_VH (20) | IL-11_Human (125) | 50-66 | 143-147 | inter-protein xl | 58 | 145 |
| Chymotrypsin | CSI-s-9B9_VH (20) | IL-11_Human (125) | 55-70 | 141-147 | inter-protein xl | 63 | 145 |
| Chymotrypsin | CSI-s-9B9_VH (20) | IL-11_Human (125) | 48-64 | 141-147 | inter-protein xl | 58 | 145 |
| Elastase | CSI-s-9B9_VK (29) | IL-11_Human (125) | 89-104 | 146-161 | inter-protein xl | 100 | 146 |

Conclusion—Epitope mapping

Using chemical cross-linking, High-Mass MALDI mass spectrometry, and nLC-Q-Exactive mass spectrometry, the molecular interface between IL-11_Human and CSI-s-9B9 was characterised.

The analysis indicates that the interaction includes the following amino acids on IL-11_Human (SEQ ID NO: 125): 30, 41, 95, 98, 145 and 146.

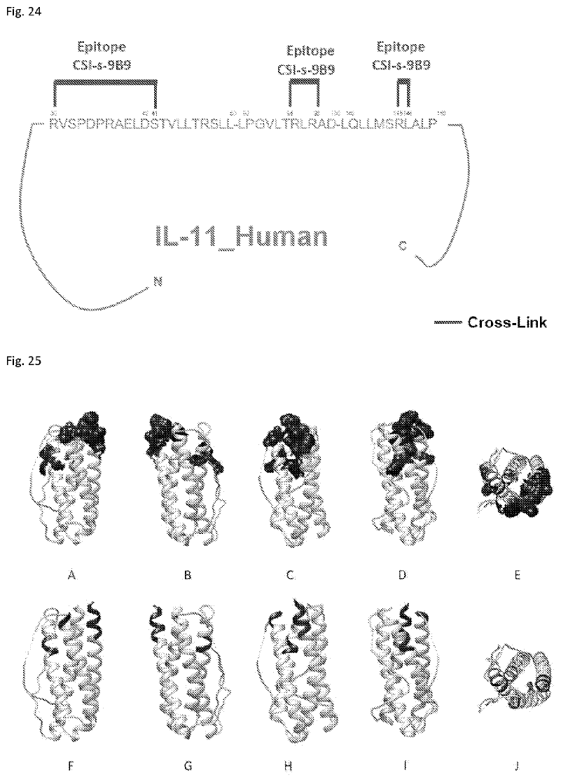

These results are illustrated in FIG. 24 and FIG. 25.

FIG. 24: Interaction CSI-s-9B9/IL-11_Human

FIG. 25: IL-11_Human/CSI-s-9B9. IL-11_Human PDB structure 4MHL was coloured in blue on the epitope site (dark shading in black & white images). Coloured IL-11_Human amino acids correspond to residues 30-41, 95-98, and 145-146 of SEQ ID NO: 125. A, B, C, D, E: ribbon/surface representation of front view (A); back view (B), side view 1 (C), side view 2 (D), and top view (E). F, G, H, I, J: ribbon representation of front view (F); back view (G), side view 1 (H), side view 2 (I), and top view (J).

Thus, the CSI-s-9B9 antibody binds to a discontinuous epitope on human IL-11, wherein the discontinuous epitope comprises amino acid residues R30, S41, T95, R98, S145, and R146 when numbered in accordance with SEQ ID NO: 125.

### 2.2. IL-11_Human/CSJ-A-s-2B11

After Trypsin, Chymotrypsin, ASP-N, Elastase, and Thermolysin proteolysis of the protein complex IL-11_Human/CSJ-A-s-2B11 with deuterated dOd12, nLC-Q-Exactive MS/MS analysis detected 15 cross-linked peptides between IL-11_Human and CSJ-A-s-2B11.

The sequences and positions of cross-links are presented in Table 91, below.

TABLE 91
CSJ-A-s-2B11 Trypsin, Chymotrypsin, ASP-N, Elastase, and Thermolysin Interlink between CSJ-A-s-
2B11 complementarity determining regions and IL-11_Human. Cross-linked peptides detected between IL-
11_Human (SEQ ID NO: 125) and CSJ-A-s-2B11 VH/VK (SEQ ID NO: 38 and SEQ ID NO: 47) complementarity
determining regions (CDRs). “nAA1” and “nAA2” are the amino acids in Protein 1
and Protein 2 respectively that are crosslinked with one another. “XLType” indicates the crosslink type.

| | Protein 1 | Protein 2 | Protein 1 | Protein 2 | | | |
|---|---|---|---|---|---|---|---|
| Enzyme | (SEQ ID NO) | (SEQ ID NO) | Position | Position | XLType | nAA1 | nAA2 |
| Elastase | CSJ-A-s-2B11_VH (38) | IL-11_Human (125) | 56-63 | 21-37 | inter-protein xl | 59 | 30 |
| Elastase | CSJ-A-s-2B11_VK (47) | IL-11_Human (125) | 49-55 | 21-37 | inter-protein xl | 52 | 30 |
| Thermolysin | CSJ-A-s-2B11_VK (47) | IL-11_Human (125) | 46-50 | 20-38 | inter-protein xl | 49 | 30 |
| Elastase | CSJ-A-s-2B11_VH (38) | IL-11_Human (125) | 30-42 | 38-43 | inter-protein xl | 35 | 41 |
| Elastase | CSJ-A-s-2B11_VK (47) | IL-11_Human (125) | 85-99 | 134-140 | inter-protein xl | 91 | 138 |
| Elastase | CSJ-A-s-2B11_VK (47) | IL-11_Human (125) | 85-98 | 134-140 | inter-protein xl | 91 | 138 |
| Elastase | CSJ-A-s-2B11_VH (38) | IL-11_Human (125) | 103-112 | 146-161 | inter-protein xl | 108 | 146 |
| Elastase | CSJ-A-s-2B11_VH (38) | IL-11_Human (125) | 92-102 | 146-161 | inter-protein xl | 97 | 146 |
| Chymotrypsin | CSJ-A-s-2B11_VK (47) | IL-11_Human (125) | 16-35 | 188-192 | inter-protein xl | 24 | 190 |
| Chymotrypsin | CSJ-A-s-2B11_VK (47) | IL-11_Human (125) | 12-30 | 188-192 | inter-protein xl | 24 | 190 |
| Chymotrypsin | CSJ-A-s-2B11_VK (47) | IL-11_Human (125) | 16-35 | 188-194 | inter-protein xl | 26 | 190 |
| Trypsin | CSJ-A-s-2B11_VH (38) | IL-11_Human (125) | 98-119 | 191-198 | inter-protein xl | 105 | 196 |
| Elastase | CSJ-A-s-2B11_VH (38) | IL-11_Human (125) | 104-113 | 195-199 | inter-protein xl | 108 | 196 |
| Elastase | CSJ-A-s-2B11_VH (38) | IL-11_Human (125) | 104-113 | 195-199 | inter-protein xl | 108 | 197 |
| Elastase | CSJ-A-s-2B11_VH (38) | IL-11_Human (125) | 30-42 | 195-199 | inter-protein xl | 35 | 198 |

### Conclusion—Epitope Mapping

Using chemical cross-linking, High-Mass MALDI mass spectrometry, and nLC-Q-Exactive mass spectrometry the molecular interface between IL-11_Human and CSJ-A-s-2B11 was characterised.

The analysis indicates that the interaction includes the following amino acids on IL-11_Human (SEQ ID NO: 125): 30, 41, 138, 146, 190, 196, 197 and 198.

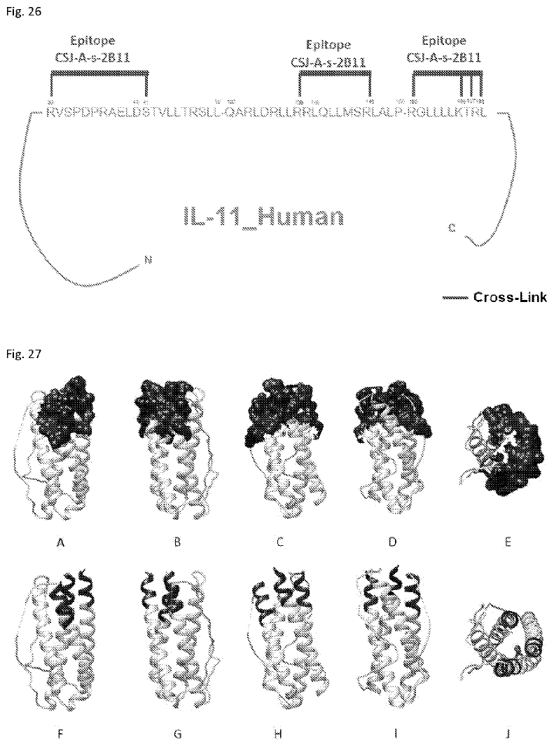

These results are illustrated in FIG. 26 and FIG. 27.

FIG. 26: Interaction CSJ-A-s-2B11/lL-11_Human

FIG. 27: IL-11_Human/CSJ-A-s-2B11. IL-11_Human PDB structure 4MHL was coloured in blue on the epitope site (dark shading on the black & white images). Coloured IL-11_Human amino acids correspond to residues 30-41, 138-146, and 190-198. A, B, C, D, E: ribbon/surface representation of front view (A); back view (B), side view 1 (C), side view 2 (D), and top view (E). F, G, H, I, J: ribbon representation of front view (F); back view (G), side view 1 (H), side view 2 (1), and top view (J).

Thus, the CSJ-A-s-2B11 antibody binds to a discontinuous epitope on human IL-11, wherein the discontinuous epitope comprises amino acid residues R30, S41, R138, R146, R190, K196, T197, and R198 when numbered in accordance with SEQ ID NO: 125.

### 2.3. IL-11_Human/CSJ-A-s-10D10

After Trypsin, Chymotrypsin, ASP-N, Elastase, and Thermolysin proteolysis of the protein complex IL-11_Human/CSJ-A-s-10D10 with deuterated d0d12, nLC-Q-Exactive MS/MS analysis detected 18 cross-linked peptides between IL-11_Human and CSJ-A-s-10D10.

The sequences and positions of cross-links are presented in the Table 92, below.

TABLE 92
CSJ-A-s-10D10 Trypsin, Chymotrypsin, ASP-N, Elastase, and Thermolysin Interlink between CSJ-A-s-
10D10 complementarity determining regions and IL-11_Human. Cross-linked peptides detected between IL-
11_Human (SEQ ID NO: 125) and CSJ-A-s-10D10 VH/VK (SEQ ID NO: 56 and SEQ ID NO: 65) complementarity
determining regions (CDRs). “nAA1” and “nAA2” are the amino acids in Protein 1
and Protein 2 respectively that are crosslinked with one another. “XLType” indicates the crosslink type.

| | Protein 1 | Protein 2 | Protein 1 | Protein 2 | | | |
|---|---|---|---|---|---|---|---|
| Enzyme | (SEQ ID NO) | (SEQ ID NO) | Position | Position | XLType | nAA1 | nAA2 |
| Elastase | CSJ-A-s-10D10_VK (65) | IL-11_Human (125) | 22-26 | 21-39 | inter-protein xl | 24 | 30 |
| Trypsin | CSJ-A-s-10D10_VH (56) | IL-11_Human (125) | 98-108 | 31-47 | inter-protein xl | 105 | 46 |
| Thermolysin | CSJ-A-s-10D10_VH (56) | IL-11_Human (125) | 100-110 | 43-48 | inter-protein xl | 105 | 46 |
| Thermolysin | CSJ-A-s-10D10_VH (56) | IL-11_Human (125) | 51-62 | 43-48 | inter-protein xl | 57 | 46 |
| Thermolysin | CSJ-A-s-10D10_VK (65) | IL-11_Human (125) | 21-32 | 43-48 | inter-protein xl | 26 | 46 |
| Trypsin | CSJ-A-s-10D10_VH (56) | IL-11_Human (125) | 44-71 | 133-139 | inter-protein xl | 57 | 135 |
| Chymotrypsin | CSJ-A-s-10D10_VH (56) | IL-11_Human (125) | 48-59 | 141-147 | inter-protein xl | 57 | 145 |
| Chymotrypsin | CSJ-A-s-10D10_VK (65) | IL-11_Human (125) | 16-33 | 141-147 | inter-protein xl | 24 | 145 |
| Trypsin | CSJ-A-s-10D10_VK (65) | IL-11_Human (125) | 25-42 | 139-146 | inter-protein xl | 26 | 145 |
| Trypsin | CSJ-A-s-10D10_VK (65) | IL-11_Human (125) | 25-42 | 139-146 | inter-protein xl | 32 | 145 |
| Trypsin | CSJ-A-s-10D10_VK (65) | IL-11_Human (125) | 25-42 | 140-146 | inter-protein xl | 34 | 145 |
| Chymotrypsin | CSJ-A-s-10D10_VH (56) | IL-11_Human (125) | 95-100 | 144-168 | inter-protein xl | 97 | 146 |
| Chymotrypsin | CSJ-A-s-10D10_VK (65) | IL-11_Human (125) | 93-98 | 143-161 | inter-protein xl | 97 | 146 |
| Chymotrypsin | CSJ-A-s-10D10_VK (65) | IL-11_Human (125) | 93-98 | 143-161 | inter-protein xl | 93 | 146 |
| Chymotrypsin | CSJ-A-s-10D10_VH (56) | IL-11_Human (125) | 46-59 | 184-192 | inter-protein xl | 57 | 190 |
| Chymotrypsin | CSJ-A-s-10D10_VH (56) | IL-11_Human (125) | 53-63 | 188-192 | inter-protein xl | 58 | 190 |
| Chymotrypsin | CSJ-A-s-10D10_VK (65) | IL-11_Human (125) | 16-32 | 184-192 | inter-protein xl | 24 | 190 |
| Chymotrypsin | CSJ-A-s-10D10_VH (56) | IL-11_Human (125) | 46-59 | 195-199 | inter-protein xl | 57 | 196 |

### Conclusion—Epitope Mapping

Using chemical cross-linking, High-Mass MALDI mass spectrometry, and nLC-Q-Exactive mass spectrometry, the molecular interface between IL-11_Human and CSJ-A-s-10D10 was characterised.

The analysis indicates that the interaction includes the following amino acids on IL-11_Human (SEQ ID NO: 125): 30, 46, 135, 145, 146, 190 and 196.

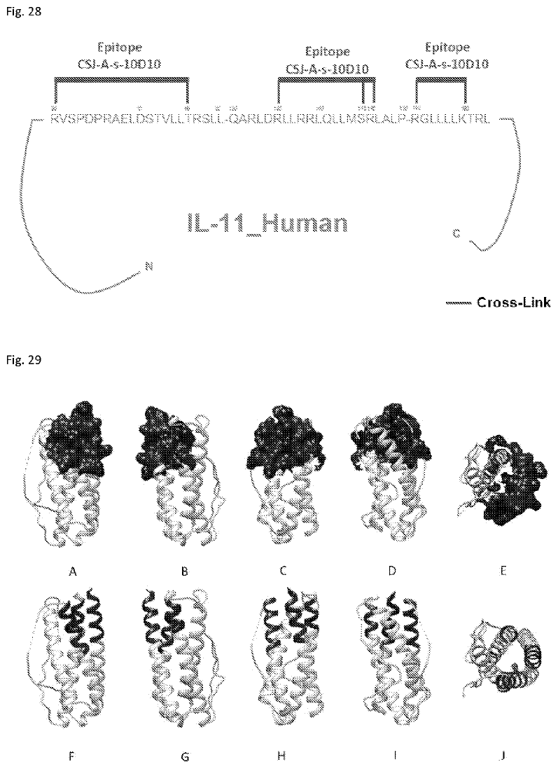

These results are illustrated in FIG. 28 and FIG. 29.

FIG. 28: Interaction CSJ-A-s-10D10/IL-11 Human

FIG. 29: IL-11_Human/CSJ-A-s-10D10. IL-11_Human PDB structure 4MHL was coloured in blue on the epitope site (dark shading in black & white images). Coloured IL-11_Human amino acids correspond to residues 30-46, 135-146, and 190-196 of SEQ ID NO: 125. A, B, C, D, E: ribbon/surface representation of front view (A); back view (B), side view 1 (C), side view 2 (D) and top view (E). F, G, H, I, J: ribbon representation of front view (F); back view (G), side view 1 (H), side view 2 (1) and top view (J).

Thus, the CSJ-A-s-10D10 antibody binds to a discontinuous epitope on human IL-11, wherein the discontinuous epitope comprises amino acid residues R30, T46, R135, S145, R146, R190, and K196 when numbered in accordance with SEQ ID NO: 125.

### 2.4. IL-11_Human/CSJ-B-s-3F6

After Trypsin, Chymotrypsin, ASP-N, Elastase, and Thermolysin proteolysis of the protein complex IL-11_Human/CSJ-B-s-3F6 with deuterated dOd12, nLC-Q-Exactive MS/MS analysis detected 19 cross-linked peptides between IL-11_Human and CSJ-B-s-3F6.

The sequences and positions of cross-links are presented in Table 93, below.

TABLE 93
CSJ-B-3F6 Trypsin, Chymotrypsin, ASP-N, Elastase, and Thermolysin Interlink between CSJ-B-3F6
complementarity determining regions and IL-11_Human. Cross-linked peptides detected between
IL-11_Human (SEQ ID NO: 125) and CSJ-B-s-3F6 VH/VK (SEQ ID NO: 2 and SEQ ID NO: 11) complementarity
determining regions (CDRs). “nAA1” and “nAA2” are the amino acids in Protein
1 and Protein 2 respectively that are crosslinked with one another. “XLType” indicates the crosslink type.

| | Protein 1 | Protein 2 | Protein 1 | Protein 2 | | | |
|---|---|---|---|---|---|---|---|
| Enzyme | (SEQ ID NO) | (SEQ ID NO) | Position | Position | XLType | nAA1 | nAA2 |
| Elastase | CSJ-B-s-3F6_VH (2) | IL11_Human (125) | 102-110 | 45-49 | inter-protein xl | 106 | 48 |
| Elastase | CSJ-B-s-3F6_VK (11) | IL11_Human (125) | 27-32 | 52-58 | inter-protein xl | 30 | 53 |
| Elastase | CSJ-B-s-3F6_VK (11) | IL11_Human (125) | 20-29 | 52-57 | inter-protein xl | 24 | 54 |
| Chymotrypsin | CSJ-B-s-3F6_VH (2) | IL11_Human (125) | 96-103 | 51-60 | inter-protein xl | 99 | 54 |
| Elastase | CSJ-B-s-3F6_VH (2) | IL11_Human (125) | 102-111 | 94-99 | inter-protein xl | 106 | 96 |
| Thermolysin | CSJ-B-s-3F6_VH (2) | IL11_Human (125) | 53-64 | 93-98 | inter-protein xl | 59 | 96 |
| Elastase | CSJ-B-s-3F6_VK (11) | IL11_Human (125) | 22-32 | 95-101 | inter-protein xl | 26 | 98 |
| Thermolysin | CSJ-B-s-3F6_VK (11) | IL11_Human (125) | 19-31 | 97-101 | inter-protein xl | 24 | 98 |
| Trypsin | CSJ-B-s-3F6_VH (2) | IL11_Human (125) | 60-66 | 97-106 | inter-protein xl | 61 | 103 |
| Thermolysin | CSJ-B-s-3F6_VH (2) | IL11_Human (125) | 27-34 | 102-110 | inter-protein xl | 30 | 106 |
| Chymotrypsin | CSJ-B-s-3F6_VK (11) | IL11_Human (125) | 16-30 | 106-110 | inter-protein xl | 24 | 107 |
| Chymotrypsin | CSJ-B-s-3F6_VK (11) | IL11_Human (125) | 12-33 | 123-133 | inter-protein xl | 24 | 128 |
| Elastase | CSJ-B-s-3F6_VK (11) | IL11_Human (125) | 20-29 | 130-136 | inter-protein xl | 24 | 132 |
| Chymotrypsin | CSJ-B-s-3F6_VK (11) | IL11_Human (125) | 55-71 | 138-142 | inter-protein xl | 55 | 138 |
| Elastase | CSJ-B-s-3F6_VK (11) | IL11_Human (125) | 86-99 | 138-142 | inter-protein xl | 91 | 139 |
| Chymotrypsin | CSJ-B-s-3F6_VK (11) | IL11_Human (125) | 12-33 | 138-142 | inter-protein xl | 24 | 139 |
| Chymotrypsin | CSJ-B-s-3F6_VH (2) | IL11_Human (125) | 97-103 | 179-183 | inter-protein xl | 100 | 182 |
| Thermolysin | CSJ-B-s-3F6_VK (11) | IL11_Human (125) | 87-99 | 189-193 | inter-protein xl | 92 | 190 |
| Chymotrypsin | CSJ-B-s-3F6_VK (11) | IL11_Human (125) | 87-92 | 186-192 | inter-protein xl | 91 | 190 |

Conclusion—Epitope mapping

Using chemical cross-linking, High-Mass MALDI mass spectrometry, and nLC-Q-Exactive mass spectrometry the molecular interface between IL-11_Human and CSJ-B-s-3F6 was characterised.

The analysis indicates that the interaction includes the following amino acids on IL-11_Human (SEQ ID NO: 125): 48, 53, 54, 96, 98, 103, 106, 107, 128, 132, 138, 139, 182 and 190.

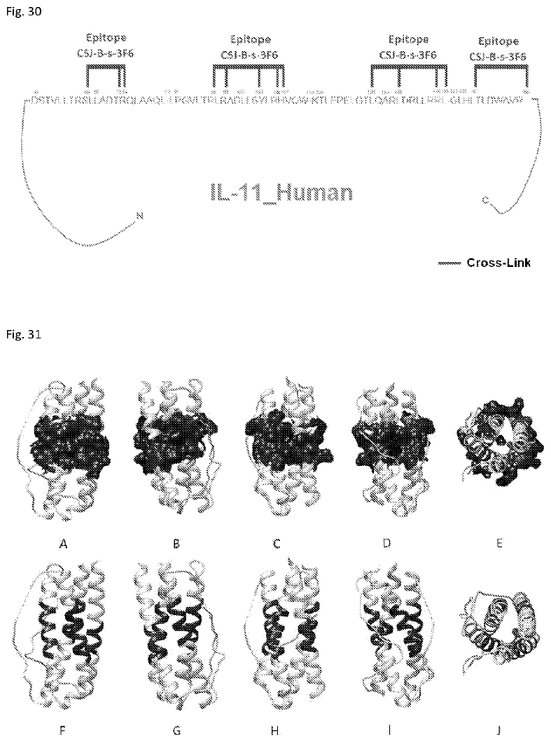

These results are illustrated in FIG. 30 and FIG. 31.

FIG. 30: Interaction CSJ-B-s-3F6/IL-11_Human FIG. 31: IL-11_Human/CSJ-B-s-3F6. IL-11_Human PDB structure 4MHL was coloured in blue on the epitope site (dark shading on the black & white images). Coloured IL-11_Human amino acids correspond to residues 48-54, 96-107, 128-139, and 182-190. A, B, C, D, E: ribbon/surface representation of front view (A); back view (B), side view 1 (C), side view 2 (D) and top view (E). F, G, H, I, i: ribbon representation of front view (F); back view (G), side view 1 (H), side view 2 (I) and top view (J).

Thus, the CSJ-B-s-3F6 antibody binds to a discontinuous epitope on human IL-11, wherein the discontinuous epitope comprises amino acid residues S48, T53, R54, R96, R98, S103, R106, H107, T128, R132. R138. R139. H182, and R190 when numbered in accordance with SEQ ID NO: 125.

### 2.5. IL-11_Human/CSJ-B-s-3G8

After Trypsin, Chymotrypsin, ASP-N, Elastase, and Thermolysin proteolysis of the protein complex IL-11_Human/CSJ-B-s-3G8 with deuterated d0d12, nLC-Q-Exactive MS/MS analysis detected 10 cross-linked peptides between IL-11_Human and CSJ-B-s-3G8.

The sequences and positions of cross-links are presented in Table 94, below.

TABLE 94
CSJ-B-s-3G8 Trypsin, Chymotrypsin, ASP-N, Elastase, and Thermolysin Interlink between CSJ-B-s-3G8
complementarity determining regions and IL-11_Human. Cross-linked peptides detected between
IL-11_Human (SEQ ID NO: 125) and CSJ-B-s-3G8 VH/VK (SEQ ID NO: 74 and SEQ ID NO: 83) complementarity
determining regions (CDRs). “nAA1” and “nAA2” are the amino acids in Protein 1
and Protein 2 respectively that are crosslinked with one another. “XLType” indicates the crosslink type.

| | Protein 1 | Protein 2 | Protein 1 | Protein 2 | | | |
|---|---|---|---|---|---|---|---|
| Enzyme | (SEQ ID NO) | (SEQ ID NO) | Position | Position | XLType | nAA1 | nAA2 |
| ASP-N | CSJ-B-s-3G8_VK (83) | IL11_Human (125) | 50-80 | 40-51 | inter-protein xl | 55 | 46 |
| ASP-N | CSJ-B-s-3G8_VK (83) | IL11_Human (125) | 81-92 | 52-61 | inter-protein xl | 91 | 54 |
| Trypsin | CSJ-B-s-3G8_VK (83) | IL11_Human (125) | 48-61 | 51-57 | inter-protein xl | 53 | 54 |
| Chymotrypsin | CSJ-B-s-3G8_VH (74) | IL11_Human (125) | 28-47 | 89-101 | inter-protein xl | 37 | 95 |
| Chymotrypsin | CSJ-B-s-3G8_VH (74) | IL11_Human (125) | 25-38 | 89-97 | inter-protein xl | 31 | 95 |
| Elastase | CSJ-B-s-3G8_VH (74) | IL11_Human (125) | 101-110 | 109-115 | inter-protein xl | 106 | 112 |
| Chymotrypsin | CSJ-B-s-3G8_VH (74) | IL11_Human (125) | 25-47 | 179-183 | inter-protein xl | 37 | 182 |
| Chymotrypsin | CSJ-B-s-3G8_VH (74) | IL11_Human (125) | 25-47 | 188-192 | inter-protein xl | 37 | 190 |
| Chymotrypsin | CSJ-B-s-3G8_VH (74) | IL11_Human (125) | 25-38 | 186-193 | inter-protein xl | 31 | 190 |
| Chymotrypsin | CSJ-B-s-3G8_VH (74) | IL11_Human (125) | 28-38 | 186-193 | inter-protein xl | 37 | 190 |

### Conclusion—Epitope Mapping

Using chemical cross-linking, High-Mass MALDI mass spectrometry, and nLC-Q-Exactive mass spectrometry, the molecular interface between IL-11_Human and CSJ-B-s-3G8 was characterised.

The analysis indicates that the interaction includes the following amino acids on IL-11_Human: 46, 54, 95, 112, 182 and 190.

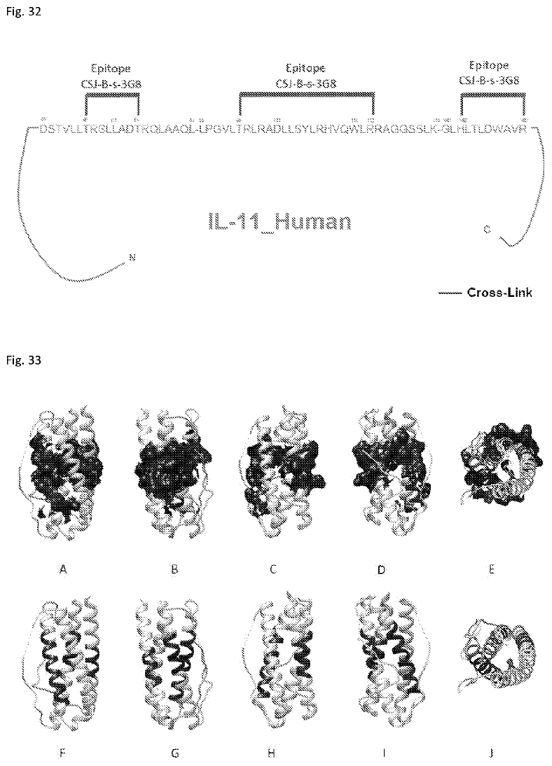

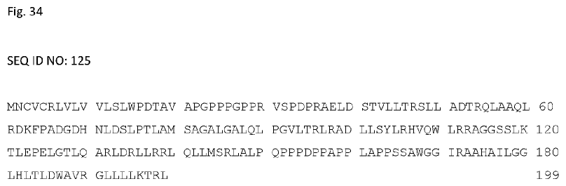

These results are illustrated in FIG. 32 and FIG. 33.

FIG. 32: Interaction CSJ-B-s-3G8/IL-11_Human

FIG. 33: IL-11_Human/CSJ-B-s-3G8. IL-11_Human PDB structure 4MHL was coloured in blue on the epitope site (dark shading in black & white images). Coloured IL-11_Human amino acids correspond to residues 46-54, 95-112, and 182-190 of SEQ ID NO: 125. A, B, C, D, E: ribbon/surface representation of front view (A); back view (B), side view 1 (C), side view 2 (D) and top view (E). F, G, H, I, J: ribbon representation of front view (F); back view (G), side view 1 (H), side view 2 (I) and top view (J).

Thus, the CSJ-B-s-3G8 antibody binds to a discontinuous epitope on human IL-11, wherein the discontinuous epitope comprises amino acid residues T46, T54, T95, R112, H182, and R190 when numbered in accordance with SEQ ID NO: 125.

### Production of Further Antibodies Specific Against Identified Epitopes

Antibodies specific against the above identified epitopes are produced as detailed below.

For a given epitope, monoclonal antibodies can be readily produced by way of animal immunisation (1, below) or library panning using display systems (2, below):

### 1. Animal Immunisation:

Animal immunisation is performed on a selected animal (mice, rat, or chicken) by either:
A. Protein-Based Immunisation: Using the IL-11 domain including the epitope, the animal is immunized (standard immunisation protocol as per Greenfield EA, “Antibodies: A Laboratory Manual”, Cold Spring Harbor Press; Second Edition, 2013, ISBN: 1936113813); or
B. Genetic Immunisation: The animal is injected with a plasmid DNA encoding expression of the IL-11 domain including the epitope. Cells of the animal will produce the protein domain, eliciting an immune response specific against the domain including the epitope.

B cells are harvested post-immunisation, and hybridomas are created using standard techniques (e.g. Greenfield EA, “Antibodies: A Laboratory Manual”, Cold Spring Harbor Press; Second Edition, 2013, ISBN: 1936113813) to produce monoclonal antibodies.

### 2. Library Panning Using Display Systems:

A. Produce antibodies suitable for subsequent panning using phage display or yeast display libraries of human antibodies (Brenda Pei Chui Song et al., “Review of phage display: Ajack-of-all-trades and master of most biomolecule display”, International Journal of Biological Macromolecules, Volume 256, Part 2, 2024, 128455, ISSN 0141-8130, PMID: 38013083, doi: 10.1016/j.ijbiomac.2023.128455 (available online 25 Nov. 2023); Weronika Jaroszewicz et al., “Phage display and other peptide display technologies”, FEMS Microbiology Reviews, Volume 46, Issue 2, March 2022, fuab052, PMID: 3467394, doi: 10.1093/femsre/fuab052)
B. “Pan” for specific displayed antibodies using the IL-11 domain including the epitope as bait.
C. Sequence the selected systems to obtain the monoclonal antibody sequences.

### 3. Identifying Correct Antibodies:

Correct antibodies are identified as follows:
A. ELISA (Enzyme-Linked Immunosorbent Assay): Antibody candidates are screened by checking their binding to the IL-11 domain including the epitope coated on a plate. A color change indicates binding.
B. Biacore (Surface Plasmon Resonance): Provides real-time data on the binding strength (affinity) and kinetics of the interaction between the antibody and the IL-11 domain including the epitope.

Based on the results obtained in these tests, antibodies with strong specific binding to the IL-11 domain including the epitope and with suitable binding kinetics are selected.

### 4. Epitope Mapping

Having produced, analysed, isolated and purified, and identified candidate antibodies, the specific epitopes on human IL-11 which they bind to are identified by epitope mapping (CovaIX AG, Zurich, Switzerland; www.covalx.com).

### 5. Antibody Selection

The results of the epitope mapping for each of the candidate antibodies is then compared to the identified epitopes as detailed above (i.e. the discontinuous epitopes which are identified as being bound by the CSI-s-9B9, CSJ-A-s-2B11, CSJ-A-s-10D10, CSJ-B-3F6, and CSJ-B-s-3G8 antibodies).

Candidate antibodies which bind the same set of epitopes on human IL-11 as those bound by any of CSI-s-9B9, CSJ-A-s-2B11, CSJ-A-s-10D10, CSJ-B-3F6, and CSJ-B-s-3G8 antibodies are then selected.

### Half-Life Determination

### Method:

The half-life (PK) of antibodies in hFcRn mice (The Jackson Laboratory, Maine, USA; www.jax.org/fcrn; Roopenian D C et al., Human FcRn transgenic mice for pharmacokinetic evaluation of therapeutic antibodies. Methods Mol Biol. 2010; 602:93-104. doi: 10.1007/978-1-60761-058-8 6. PMID: 20012394) was determined by injecting (IV) a single dose of antibody at 2 mg/kg and 10 mg/kg dosing. The concentration of the antibody in the blood of mice (n=4) was assessed using the ELISA method with a goat anti-human IgG coating, a goat anti-human IgG-biotin secondary antibody and detected with SA-HRP (streptavidin-horse radish peroxidase)+ TMB reading (450-540 nm). Analysis was done with SoftMax (RTM) (Molecular Devices LLC, CA, USA; www.moleculardevices.com) and WinNonlin (RTM) (Certara Inc., NJ, USA; www.certara.com).

### Results:

The clone CVH4 (D54E)/CVL3+YTE had a half-life of 501h (2 mg/kg) and 234h (10 mg/kg). The clone GVH5 (D54E)/CVL3+YTE had a half-life of 594h (2 mg/kg) and 543h (10 mg/kg). In both cases, the YTE mutation improved the half-life of the antibodies.

### Further Experiments

These two antibodies were also assessed for their ability to induce CD4+ T cell responses using a time course assay. PBMCs (human peripheral blood mononuclear cells) from a cohort of 50 healthy donors representing the European and North American population (based on HLA allotypes) were incubated with the samples. Responses were measured using proliferation assays ([3H]-Thymidine uptake).

Analysis of the frequency and magnitude of the responses show that both samples induced a similar proliferation response to Herceptin, the low immunogenicity control, and are therefore considered to have a low risk of immunogenicity in the clinic.

Following from the above description and invention summaries, it should be apparent to those of ordinary skill in the art that, while the methods and apparatuses herein described constitute exemplary embodiments of the present invention, the invention contained herein is not limited to this precise embodiment and that changes may be made to such embodiments without departing from the scope of the invention as defined by the claims. Additionally, it is to be understood that the invention is defined by the claims and it is not intended that any limitations or elements describing the exemplary embodiments set forth herein are to be incorporated into the interpretation of any claim element unless such limitation or element is explicitly stated. Likewise, it is to be understood that it is not necessary to meet any or all of the identified advantages or objects of the invention disclosed herein in order to fall within the scope of any claims, since the invention is defined by the claims and since inherent and/or unforeseen advantages of the present invention may exist even though they may not have been explicitly discussed herein.

1. An antibody that binds to human IL-11, the antibody comprising a heavy chain (HC) variable region (VH) sequence and a light chain (LC) variable region (VL) sequence,
(i) the heavy chain variable region sequence comprising the CDR3 and at least one additional CDR selected from the group consisting of:
(a) HC-CDR1 comprising the amino acid sequence GFSLSTSGMV (SEQ ID NO: 4) or a variant having one, two or three amino acid substitutions therein,
(b) HC-CDR2 comprising the amino acid sequence of IDWXGEK wherein ‘X’ is D (SEQ ID NO. 105) or E (SEQ ID NO: 123) or a variant having one or two amino acid substitutions therein, and
(c) HC-CDR3 comprising the amino acid sequence ARSGNYFEY (SEQ ID NO: 8) or a variant having one or two amino acid substitutions therein; and
(ii) the light chain variable region sequence comprising the CDR3 and at least one additional CDR selected from the group consisting of:
(a) LC-CDR1 comprising the amino acid sequence EDIYNA (SEQ ID NO: 13) or a variant having one or two amino acid substitutions therein,
(b) LC-CDR2 comprising the amino acid sequence DAN (SEQ ID NO: 15) or a variant having one amino acid substitution therein, and
(c) LC-CDR3 comprising the amino acid sequence QQYYDYPNT (SEQ ID NO: 17) or a variant having one or two amino acid substitutions therein.

2. An antibody according to claim 1, wherein the heavy chain variable region sequence additionally comprises at least one of:
(a) FWR1 comprising the amino acid sequence QVILKESGPGMLQPSKTLSLTCSFS (SEQ ID NO: 3) or a sequence having at least 50% sequence identity therewith,
(b) FWR2 comprising the amino acid sequence VSWIRQPSGKSLEWLAA (SEQ ID NO: 5) or a sequence having at least 50% sequence identity therewith,
(c) FWR3 comprising the amino acid sequence YYNPSLKSRLTVSKDTSNTQVFLQITTVDIADTATYYC (SEQ ID NO: 7) or a sequence having at least 50% sequence identity therewith, and
(d) FWR4 comprising the amino acid sequence WGQGVIVXVSS (SEQ ID NO: 9) or a sequence having at least 50% sequence identity therewith.

3. An antibody according to claim 1, wherein the light chain variable region sequence additionally comprises at least one of:
(a) FWR1 comprising the amino acid sequence DNQMTQSPASLSASLGETVTIECRTS (SEQ ID NO: 12) or a sequence having at least 50% sequence identity therewith,
(b) FWR2 comprising the amino acid sequence LAWYQQKPGKSPQLLIY (SEQ ID NO:
14) or a sequence having at least 50% sequence identity therewith,
(c) FWR3 comprising the amino acid sequence RLHIGVPSRFSGSGSGTQFSLKINSLQSEDVASYFC (SEQ ID NO: 16) or a sequence having at least 50% sequence identity therewith, and
(d) FWR4 comprising the amino acid sequence FGAGTKLELN (SEQ ID NO: 18) or a sequence having at least 50% sequence identity therewith.

4. An antibody according to claim 1, wherein the HC-CDR2 has the sequence IDWEGEK (SEQ ID NO: 105).

5. An antibody that binds to human IL-11, the antibody comprising a heavy chain (HC) variable region sequence and a light chain (LC) variable region sequence,
(i) the heavy chain variable region sequence comprising the CDR3 and at least one additional CDR selected from the group consisting of:
(a) HC-CDR1 comprising the amino acid sequence GYTFTSYV (SEQ ID NO: 22) or a variant having one, two or three amino acid substitutions therein,
(b) HC-CDR2 comprising the amino acid sequence IYPYNDDT (SEQ ID NO: 24) or a variant having one or two amino acid substitutions therein, and
(c) HC-CDR3 comprising the amino acid sequence TRERATYGDLAWFAY (SEQ ID NO: 26) or a variant having one or two amino acid substitutions therein; and
(ii) the light chain variable region sequence comprising the CDR3 and at least one additional CDR selected from the group consisting of:
(a) LC-CDR1 comprising the amino acid sequence ENIDSYGNSF (SEQ ID NO: 31) or a variant having one or two amino acid substitutions therein,
(b) LC-CDR2 comprising the amino acid sequence LAS or a variant having one amino acid substitution therein, and
(c) LC-CDR3 comprising the amino acid sequence QQNNEDPYT (SEQ ID NO: 35) or a variant having one or two amino acid substitutions therein, or
(i) the heavy chain variable region sequence comprising the CDR3 and at least one additional CDR selected from the group consisting of:
(a) HC-CDR1 comprising the amino acid sequence GFSLDNYN (SEQ ID NO: 40) or a variant having one, two or three amino acid substitutions therein,
(b) HC-CDR2 comprising the amino acid sequence IWTDGIT (SEQ ID NO: 42) or a variant having one or two amino acid substitutions therein, and
(c) HC-CDR3 comprising the amino acid sequence ARSYYYSLYYFDY (SEQ ID NO: 44) or a variant having one or two amino acid substitutions therein; and
(ii) the light chain variable region sequence comprising the CDR3 and at least one additional CDR selected from the group consisting of:
(a) LC-CDR1 comprising the amino acid sequence EDIYSN (SEQ ID NO: 49) or a variant having one or two amino acid substitutions therein,
(b) LC-CDR2 comprising the amino acid sequence DAH or a variant having one amino acid substitution therein, and
(c) LC-CDR3 comprising the amino acid sequence QQYDNYNT (SEQ ID NO: 53) or a variant having one or two amino acid substitutions therein, or
(i) the heavy chain variable region sequence comprising the CDR3 and at least one additional CDR selected from the group consisting of:
(a) HC-CDR1 comprising the amino acid sequence GFSLTNYH (SEQ ID NO: 58) or a variant having one, two or three amino acid substitutions therein,
(b) HC-CDR2 comprising the amino acid sequence MWIDGDT (SEQ ID NO: 60) or a variant having one or two amino acid substitutions therein, and
(c) HC-CDR3 comprising the amino acid sequence AREGLNWFAY (SEQ ID NO: 62) or a variant having one or two amino acid substitutions therein; and
(ii) the light chain variable region sequence comprising the CDR3 and at least one additional CDR selected from the group consisting of:
(a) LC-CDR1 comprising the amino acid sequence QGIDDY (SEQ ID NO: 67) or a variant having one or two amino acid substitutions therein,
(b) LC-CDR2 comprising the amino acid sequence DVT or a variant having one amino acid substitution therein, and
(c) LC-CDR3 comprising the amino acid sequence LQSYSTPYT (SEQ ID NO: 71) or a variant having one or two amino acid substitutions therein, or
(i) the heavy chain variable region sequence comprising the CDR3 and at least one additional CDR selected from the group consisting of:
(a) HC-CDR1 comprising the amino acid sequence GFSMSTSGMV (SEQ ID NO: 76) or a variant having one, two or three amino acid substitutions therein,
(b) HC-CDR2 comprising the amino acid sequence IDWDGEK (SEQ ID NO: 78) or a variant having one or two amino acid substitutions therein, and
(c) HC-CDR3 comprising the amino acid sequence ARSGNYFDY (SEQ ID NO: 80) or a variant having one or two amino acid substitutions therein; and
(ii) the light chain variable region sequence comprising the CDR3 and at least one additional CDR selected from the group consisting of:
(a) LC-CDR1 comprising the amino acid sequence EDIYNG (SEQ ID NO: 85) or a variant having one or two amino acid substitutions therein,
(b) LC-CDR2 comprising the amino acid sequence DAN or a variant having one amino acid substitution therein, and
(c) LC-CDR3 comprising the amino acid sequence QQYYDYPNT (SEQ ID NO: 89) or a variant having one or two amino acid substitutions therein.

6. An antibody according to claim 5, wherein the heavy chain variable region sequence additionally comprises at least one of:
(a) FWR1 comprising the amino acid sequence EVHLQQSGPELVKPGASVKMSCKAS (SEQ ID NO: 21) or a sequence having at least 50% sequence identity therewith,
(b) FWR2 comprising the amino acid sequence IHWVKQRPGQGLEWIGN (SEQ ID NO: 23) or a sequence having at least 50% sequence identity therewith,
(c) FWR3 comprising the amino acid sequence NYNEKFKGKATLTSDKSSSTAYMELSSLTSEESAVYFC (SEQ ID NO: 25) or a sequence having at least 50% sequence identity therewith, and
(d) FWR4 comprising the amino acid sequence WGQGTLVTVSA (SEQ ID NO: 27) or a sequence having at least 50% sequence identity therewith.

7. An antibody according to claim 5, wherein the light chain variable region sequence additionally comprises at least one of:
(a) FWR1 comprising the amino acid sequence NIVLTQSPASLAVSLGQRATISCRAS (SEQ ID NO: 30) or a sequence having at least 50% sequence identity therewith,
(b) FWR2 comprising the amino acid sequence MHWYQQKPGQPPKLLIY (SEQ ID NO: 32) or a sequence having at least 50% sequence identity therewith,
(c) FWR3 comprising the amino acid sequence ILESGVPARFSGSGSRTDFTLTIDPVEADDAATYYC (SEQ ID NO: 34) or a sequence having at least 50% sequence identity therewith, and
(d) FWR4 comprising the amino acid sequence FGGGTKLEIK (SEQ ID NO: 36) or a sequence having at least 50% sequence identity therewith.

8. (canceled)

9. An antibody according to claim 5, wherein the heavy chain variable region sequence additionally comprises at least one of:
(a) FWR1 comprising the amino acid sequence QVQLKESGPGLVQPSQTLSLTCTVS (SEQ ID NO: 39) or a sequence having at least 50% sequence identity therewith,
(b) FWR2 comprising the amino acid sequence VHWVRQPTGGGLEWLGG (SEQ ID NO: 41) or a sequence having at least 50% sequence identity therewith,
(c) FWR3 comprising the amino acid sequence DYTSPLRSRLSISRDTSKSQVFLKMKSLQTEDIATYYC (SEQ ID NO: 43) or a sequence having at least 50% sequence identity therewith, and
(d) FWR4 comprising the amino acid sequence WGQGVMVTVSS (SEQ ID NO: 45) or a sequence having at least 50% sequence identity therewith.

10. An antibody according to claim 5, wherein the light chain variable region sequence additionally comprises at least one of:
(a) FWR1 comprising the amino acid sequence DIQMTQSPASLSASLGETVTIECRAS (SEQ ID NO: 48) or a sequence having at least 50% sequence identity therewith,
(b) FWR2 comprising the amino acid sequence LAWYQQKPGDSPQLLIY (SEQ ID NO: 50) or a sequence having at least 50% sequence identity therewith,
(c) FWR3 comprising the amino acid sequence RLAEGVPSRFSGSGSGTQYSLKINSLHSEDVASYFC (SEQ ID NO: 52) or a sequence having at least 50% sequence identity therewith, and
(d) FWR4 comprising the amino acid sequence FGVGTKLDLK (SEQ ID NO: 54) or a sequence having at least 50% sequence identity therewith.

11. (canceled)

12. An antibody according to claim 5, wherein the heavy chain variable region sequence additionally comprises at least one of:
(a) FWR1 comprising the amino acid sequence QVQLKESGPGLVQPSQTLSLTCTVS (SEQ ID NO: 57) or a sequence having at least 50% sequence identity therewith,
(b) FWR2 comprising the amino acid sequence VHWVRQPPGKGLEWMGV (SEQ ID NO: 59) or a sequence having at least 50% sequence identity therewith,
(c) FWR3 comprising the amino acid sequence SYNSALRSRLSISRDTSKSQVFLKMSSLQTEDTATYYC (SEQ ID NO: 61) or a sequence having at least 50% sequence identity therewith, and
(d) FWR4 comprising the amino acid sequence WGRGTLVTVSS (SEQ ID NO: 63) or a sequence having at least 50% sequence identity therewith.

13. An antibody according to claim 5, wherein the light chain variable region sequence additionally comprises at least one of:
(a) FWR1 comprising the amino acid sequence DIQMTQSPASLSVSLEEVVTITCKAS (SEQ ID NO: 66) or a sequence having at least 50% sequence identity therewith,
(b) FWR2 comprising the amino acid sequence LSWYQQKPGKSPQLLIS (SEQ ID NO: 68) or a sequence having at least 50% sequence identity therewith,
(c) FWR3 comprising the amino acid sequence SLADGVPSRFSGSRSGTQYSLKISRPQVDDSGIYYC (SEQ ID NO: 70) or a sequence having at least 50% sequence identity therewith, and
(d) FWR4 comprising the amino acid sequence FGAGTKLELK (SEQ ID NO: 72) or a sequence having at least 50% sequence identity therewith.

14. (canceled)

15. An antibody according to claim 5, wherein the heavy chain variable region sequence additionally comprises at least one of:
(a) FWR1 comprising the amino acid sequence QVTLKESGPGMLQPSKTVSLTCSFS (SEQ ID NO: 75) or a sequence having at least 50% sequence identity therewith,
(b) FWR2 comprising the amino acid sequence VSWIRQPSGKSLEWLAA (SEQ ID NO: 77) or a sequence having at least 50% sequence identity therewith,
(c) FWR3 comprising the amino acid sequence YYNPSLKSRLTVSKDTSNTQVFLKISSVDIADSATYYC (SEQ ID NO: 79) or a sequence having at least 50% sequence identity therewith, and
(d) FWR4 comprising the amino acid sequence WGQGVMVTVSS (SEQ ID NO: 81) or a sequence having at least 50% sequence identity therewith.

16. An antibody according to claim 5, wherein the light chain variable region sequence additionally comprises at least one of:
(a) FWR1 comprising the amino acid sequence DIQMTQSPASLSASLGETVTIECRAS (SEQ ID NO: 84) or a sequence having at least 50% sequence identity therewith,
(b) FWR2 comprising the amino acid sequence LVWYQQKPGKSPQLLIY (SEQ ID NO: 86) or a sequence having at least 50% sequence identity therewith,
(c) FWR3 comprising the amino acid sequence RLHTGVPSRFSGGGSGTQYSLKINSLQSEDVASYFC (SEQ ID NO: 88) or a sequence having at least 50% sequence identity therewith, and
(d) FWR4 comprising the amino acid sequence FGAGTKLELK (SEQ ID NO: 90) or a sequence having at least 50% sequence identity therewith.

17. (canceled)

18. An antibody according to claim 1, wherein the heavy chain variable region sequence comprises all three of the CDR1, CDR2 and CDR3.

19. An antibody according to claim 1, wherein the light chain variable region sequence comprises all three of the CDR1, CDR2 and CDR3.

20. An antibody according to claim 1, wherein it comprises a heavy chain having the sequence of SEQ ID NO: 99 and a light chain having the sequence of SEQ ID NO: 94.

21. An antibody according to claim 1, wherein it comprises a heavy chain having the sequence of SEQ ID NO: 100 and a light chain having the sequence of SEQ ID NO: 94.

22. An antibody that binds to human IL-11, wherein the antibody binds to a discontinuous epitope on human IL-11, wherein the discontinuous epitope comprises amino acid residues:
(a) R30, S41, T95, R98, S145, and R146;
(b) R30, S41, R138, R146, R190, K196, T197, and R198;
(c) R30, T46, R135, S145, R146, R190, and K196;
(d) S48, T53, R54, R96, R98, S103, R106, H107, T128, R132, R138, R139, H182, and R190; or
(e) T46, T54, T95, R112, H182, and R190;
when numbered in accordance with SEQ ID NO: 125.

23. An antibody according to claim 1, wherein it is a monoclonal antibody.

24. An antibody according to claim 1, wherein it is chemically or biologically modified.

25. An antibody according to claim 24, wherein it is glycosylated, PEGylated or HESylated.

26-41. (canceled)

42. A method comprising the steps of:
i) reacting an antibody according to claim 1, specific to the protein with serum from a patient; and
ii) detecting any antibody-antigen binding reaction.
